(19) United States
(12) Patent Application Publication
(10) Pub. No.: US 2026/0176598 A1
(43) Pub. Date: Jun. 25, 2026

(54) EXPRESSION CONSTRUCT

(71) Applicant: QUELL THERAPEUTICS LIMITED, London (GB)

(72) Inventor: Jenny MCGOVERN, London (GB)

(21) Appl. No.: 19/259,737

(22) Filed: Jul. 3, 2025

(30) Foreign Application Priority Data
Jul. 4, 2024 (GB) 2409730.5
Jun. 20, 2025 (GB) 2509833.6

(51) Int. Cl. C12N 9/12; A61K 40/11; A61K 40/31; A61K 40/41; C07K 14/47; C07K 16/40; C12N 5/0783; C12N 15/86

(52) U.S. Cl. CPC C12N 9/1217; A61K 40/11; A61K 40/31; A61K 40/416; A61K 40/418; C07K 14/4702; C07K 16/40; C12N 5/0637; C12N 15/86; C12Y 207/02003; C12Y 306/01005; C12N 2510/00; C12N 2740/15043

(57) ABSTRACT

An expression construct, a nucleic acid or vector or cell comprising said expression construct, and various uses of said expression construct, nucleic acid, vector or cell is disclosed herein. Particularly, an expression construct comprising a PGK promoter operably linked to (i) a first nucleotide sequence encoding a chimeric antigen receptor (CAR), and (ii) a second nucleotide sequence encoding a FOXP3 polypeptide, wherein the first nucleotide sequence is located upstream of the second nucleotide sequence, is provided.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Jul. 2, 2025, is entitled “173267-04 Sequencelisting.xml”, and is 292,772 bytes in size.

### TECHNICAL FIELD

The present disclosure relates generally to the field of expression constructs, nucleic acid molecules, and vectors useful to express polypeptides in cells, particularly in immune cells useful in adoptive cell therapy (ACT). The disclosure provides expression constructs comprising a phosphoglycerate kinase 1 (PGK) promoter operably linked to a first nucleotide sequence encoding a chimeric antigen receptor (CAR) and a second nucleotide sequence encoding a FOXP3 polypeptide, wherein the first nucleotide sequence is located upstream of the second nucleotide sequence. The disclosure further provides nucleic acid molecules, vectors and cells comprising said expression constructs, particularly immune cells such as T cells, particularly regulatory T cells (Tregs). A method of making said cells is also provided. The cells, particularly Tregs, may be used for inducing tolerance to a transplant, treating and/or preventing graft-versus-host disease (GvHD), treating or preventing an infectious, allergic, autoimmune or inflammatory disease, or for inducing immunosuppression, or for promoting tissue repair and/or tissue regeneration.

### BACKGROUND

Immunotherapy is emerging as a beneficial tool for the treatment of many conditions, ranging from cancer, autoimmune and inflammatory diseases, to the prevention of solid organ transplant rejection. In particular, there has been increased clinical activity in the area of adoptive cellular immunotherapy (ACT), particularly in regulatory T cell (Treg) cell therapies, across the autoimmune and inflammatory space.

CD4+FOXP3+ regulatory T cells (Tregs) are a lymphocyte subset that is essential for the maintenance of dominant immunological tolerance by inhibiting the function of various effector immune cell subsets such as T effector cells. In addition, Tregs are also known to promote tissue repair and regeneration. Tregs are able to confer immune tolerance through multiple contact-dependent and independent mechanisms. These include production of anti-inflammatory soluble mediators such as IL-10, TGF-β and IL-35, consumption of IL-2, expression of negative regulatory cell surface receptors, such as CTLA-4, and targeting T cells directly or indirectly through APCs. Importantly, once activated, Tregs can suppress immune responses in a non-antigen specific fashion (bystander suppression), i.e., once activated, Tregs have the ability to modulate the local immune micro-environment and suppress inflammation. Furthermore, they can confer a suppressive phenotype on other cells of the immune system, a process called “infectious tolerance”.

Artificial chimeric antigen receptors (CARs) have been used to confer antigen specificity on a cell. CARs are generally composed of an extracellular antigen-binding domain (e.g. a scFv specific for the target antigen), a transmembrane domain, and an intracellular signalling domain which sends signals into the cell and activates it upon binding of the antigen to the extracellular antigen-binding domain. This enables the cells to provide a targeted response, for example CAR-Treg cells are able to provide a targeted immune suppression. CAR-Tregs have been reported to have increased suppressive ability compared to polyclonal Tregs.

Treg cells express FOXP3 and conventional T cells (Tcon cells) can be differentiated towards a regulatory phenotype ex vivo by expressing FOXP3 in those cells. Loss of FOXP3 expression is associated with a loss of suppressive function in regulatory T cells and a potential return to an effector phenotype. It has therefore been proposed to express exogenous FOXP3 in immune cells to provide a stable Treg phenotype or to confer a Treg phenotype on the cell.

Expression of transgenes in a cell can be obtained by introducing one or more expression constructs encoding the transgenes into the cell. The expression constructs may be comprised within a vector, particularly a viral vector, to enable it to be introduced into a cell. The transgenes may be operably linked to one or more exogenous promoters which lead to the initiation of transcription of the transgenes. Although a large number of promoters are known in the art and have been used to express transgenes in target cells, many of these promoters are viral in nature which have been reported in the literature to be more prone to gene silencing. Particularly for clinical engineered cell products, to avoid such issues, selection of a mammalian promoter may be desirable in certain circumstances. However, mammalian promoters typically are weaker than viral promoters and may not provide vector titre levels or gene expression levels which are necessary to allow production of a dose for a clinical product. Further, other variables, including the number, type and size of transgenes to be expressed, construct design, the formation of secondary structures and the type of cell in which expression is desired, may all affect the ability of a particular promoter to provide desired expression levels of one or more transgenes, and of an expression construct to be packaged with a desirable titre in a viral vector. It can therefore be challenging to select a mammalian promoter and construct design which provides sufficient transgene expression and for viral vector production, sufficient vector titre. It may be particularly difficult to select a suitable promoter to obtain the desired level of transgene expression in cells which are less metabolically active such as Tregs.

### SUMMARY

The present inventors have developed expression constructs for co-expression of a CAR and an exogenous FOXP3 polypeptide in cells, which are operably linked to a mammalian promoter, namely a phosphoglycerate kinase 1 (PGK) promoter. These nucleic acid molecules are particularly suitable for co-expression of a CAR and exogenous FOXP3 in regulatory T cells (Tregs). Surprisingly, the inventors found that the mammalian PGK promoter, when combined with a nucleotide sequence encoding a CAR and FOXP3, is able to provide desirable levels of viral vector titre, and is able to drive desirable levels of cell transduction/transgene expression, particularly desirable expression of a CAR and a FOXP3 polypeptide in Tregs, whereas other mammalian promoters were not. Additionally, the inventors identified that gene order of CAR and FOXP3 had an unexpected positive impact on the production of viral vector and the transduction of cells, particularly for bicistronic constructs. Further, the inventors found that Tregs transduced with the expression construct of the invention were able to efficiently expand and were at least functionally equivalent or highly similar to Tregs transduced with an expression construct driving co-expression of a CAR and a FOXP3 polypeptide using a viral SFFV promoter, despite the Tregs transduced with the PGK construct having a lower CAR MFI than the Tregs transduced with the SFFV promoter construct. In addition, the inventors found that greater Treg expansion was obtained with Tregs transduced with the PGK construct compared to Tregs transduced with the SFFV promoter construct. Thus, the inventors have identified a new expression construct which utilises a mammalian promoter that is capable of being packaged as a viral vector with desirable titres, resulting in efficient transduction and expansion of Treg cells and expression of CAR and FOXP3 transgenes. The new expression construct design is particularly advantageous for the production and use of a cell therapy product, where vector titre, cell transduction and transgene expression, and cell expansion are required at particular levels to enable the production of a dose of cell product.

Accordingly, in one aspect, the present invention provides an expression construct comprising a PGK promoter operably linked to:
(i) a first nucleotide sequence encoding a chimeric antigen receptor (CAR); and
(ii) a second nucleotide sequence encoding a FOXP3 polypeptide;
wherein the first nucleotide sequence is located upstream of the second nucleotide sequence.

The region that lies towards the 5′ end of the DNA coding strand is referred to as “upstream”. Therefore, in other words, the present invention provides an expression construct comprising 5′ to 3′:
a PGK promoter operably linked to;
   (i) a first nucleotide sequence encoding a chimeric antigen receptor (CAR); and
   (ii) a second nucleotide sequence encoding a FOXP3 polypeptide.

The first and second nucleotide sequences are operably linked to the same PGK promoter. By “operably linked to the same promoter” it is meant that there is a functional linkage between the PGK promoter and both the first and second nucleotide sequence that results in expression of the first and second nucleotide sequences. In other words, transcription of the first and second nucleotide sequences is initiated from the same PGK promoter and that the nucleotide sequences are positioned and oriented for transcription to be initiated from the promoter. Polynucleotides operably linked to a promoter are under transcriptional regulation of that promoter.

In certain embodiments, the expression construct does not comprise any other coding nucleotide sequence or, alternatively put, does not comprise any other coding nucleotide sequence operably linked to the same PGK promoter. In other words, the expression construct defined herein may be bicistronic because it encodes two discrete proteins (the CAR and the FOXP3 polypeptide encoded by the first and second nucleotide sequences). By “coding sequence” or “coding region” it is meant the nucleotide sequence that is translated and thus codes for a discrete protein.

The polypeptides may be produced in a cell as individual components, i.e., as discrete entities. Thus, the CAR and FOXP3 polypeptides may be separately located, in or on the cell in which they are expressed, as separate and distinct, or discrete, functional polypeptides. In certain embodiments, this may be achieved by the presence of cleavage sequences in the nucleic acid molecule, in particular self-cleaving sequences, in between the first nucleotide sequence and second nucleotide sequence and/or by the presence of an internal ribosome entry site (IRES) between the first nucleotide sequence and second nucleotide sequence. The self-cleavage sequence may be a 2A sequence, optionally selected from P2A, T2A, E2A and F2A. For example, the self-cleavage sequence may be the T2A sequence.

As used herein, the term “PGK promoter” refers to a promoter that is derived from an endogenous promoter operably linked to an endogenous PGK gene in an unmodified or non-engineered cell. Alternatively viewed, the PGK promoter as used herein is derived from an endogenous, wildtype or naturally occurring PGK promoter. An endogenous, wildtype or naturally occurring PGK promoter is capable initiating transcription of an endogenous, naturally occurring or wildtype PGK gene. The PGK gene may be the human PGK gene. The PGK promoter as referred to herein may comprise or consist of an identical sequence to an endogenous, wildtype or naturally occurring PGK promoter or may be a functional variant or functional fragment thereof.

The PGK promoter may, for example, comprise, consist essentially of or consist of the sequence of SEQ ID NO: 195 or a functional variant or functional fragment thereof.

By “functional variant” it is meant any variant that is able to function as a promoter (i.e., initiate transcription and result in expression of the transgene(s) to which it is operably linked). Variants therefore include PGK promoters that include one or more nucleotide truncations, deletions, substitutions or insertions. By “functional fragment” it is meant a fragment of a reference PGK promoter (e.g. a fragment of an endogenous, wildtype or naturally occurring PGK promoter or a fragment of SEQ ID NO: 195) which is able to function as a promoter (i.e. initiate transcription and result in expression of the transgene to which it is operably linked). A functional fragment may further comprise one or more nucleotide deletions, substitutions or insertions.

The functional variant may, for example, have at least about 70% sequence identity to an endogenous, wildtype or naturally occurring PGK promoter or to for example, SEQ ID NO: 195. For example, the functional variant may have at least about 75% or at least about 80% or at least about 85% or at least about 90% or at least about 91% or at least about 92% or at least about 93% or at least about 94% or at least about 95% or at least about 96% or at least about 97% or at least about 98% or at least about 99% sequence identity to an endogenous, wildtype or naturally occurring PGK promoter or to for example, SEQ ID NO: 195.

A functional fragment may have at least 20% sequence identity to an endogenous, wildtype or naturally occurring PGK promoter or to for example, SEQ ID NO. 195. For example, the functional fragment may have at least about 30% or at least about 40%, or at least about 50%, or at least about 60%, or at least about 70%, or at least about 80%, or at least about 85%, or at least about 90%, or at least about 95%, or at least about 96%, or at least about 97%, or at least about 98%, or at least about 99% sequence identity to an endogenous, wildtype or naturally occurring PGK promoter or to for example, SEQ ID NO: 195. As discussed above, a functional fragment may further comprise one or more nucleotide deletions, substitutions or insertions, as compared to an endogenous, wildtype or naturally occurring PGK promoter or to for example, SEQ ID NO. 195, however, the nucleotide sequence of the fragment should have at least 70% (e.g. at least 80, 85, 90, 95, 96, 97, 98 or 99%) sequence identity to its corresponding endogenous, wildtype or naturally occurring PGK promoter fragment.

Alternatively viewed, a functional fragment may be referred to as a truncated PGK promoter. Therefore, in certain embodiments, the PGK promoter comprises, consists essentially of or consists of a truncated PGK promoter or a functional variant thereof, for example a truncated human PGK promoter or a functional variant thereof. In certain embodiments, the PGK promoter is a truncated human PGK promoter or a functional variant thereof. By “truncated” it is meant that a portion at the 3′ and/or 5′ end of the PGK promoter has been removed (deleted) such that the nucleotide sequence of the truncated PGK promoter is shorter than the reference PGK promoter. The reference PGK promoter may be an endogenous, wildtype or naturally occurring PGK promoter or SEQ ID NO: 195 or nucleotides 1 to 531 of SEQ ID NO: 274. The truncated PGK promoter may, for example, comprise, consist essentially of or consist of the sequence of SEQ ID NO: 196, SEQ ID NO: 197, SEQ ID NO: 198 or SEQ ID NO: 199.

The functional variant of the truncated PGK promoter may, for example, have at least about 70% sequence identity to a reference truncated PGK promoter. For example, the variant may have at least about 75% or at least about 80% or at least about 85% or at least about 90% or at least about 95% or at least about 96% or at least about 97% or at least about 98% or at least about 99% sequence identity to a reference truncated PGK promoter. The reference truncated PGK promoter may, for example, be SEQ ID NO: 196, SEQ ID NO: 197, SEQ ID NO: 198 or SEQ ID NO: 199.

In certain embodiments, the CAR is a human CAR. By “human CAR” it is meant that all domains of the CAR (e.g. hinge, transmembrane, co-stimulatory and/or signalling domains) are all derived from human proteins.

The CAR may comprise an exodomain comprising an antigen recognition domain. The antigen recognition domain may comprise means for specifically binding a target antigen, The antigen recognition domain may specifically bind to ENTPD3. The antigen recognition domain may comprise means for specifically binding to ENTPD3. The antigen recognition domain may be an antibody, an antibody fragment, or derived from an antibody. For example, the antigen recognition domain may be a single chain antibody (scFv).

In certain embodiments, the antigen recognition domain comprises:
i. VH CDR1, 2 and 3 sequences set forth in SEQ ID NOs: 7, 8 and 9 respectively and VL CDR1, 2 and 3 sequences set forth in SEQ ID NOs: 10, 11 and 12 respectively;
ii. VH CDR1, 2 and 3 sequences set forth in SEQ ID NOs: 19, 20 and 21 respectively and VL CDR1, 2 and 3 sequences set forth in SEQ ID NOs: 22, 23 and 24 respectively;
iii. VH CDR1, 2 and 3 sequences set forth in SEQ ID NOs: 73, 74 and 75 respectively and VL CDR1, 2 and 3 sequences set forth in SEQ ID NOs: 76, 77 and 78 respectively;
iv. VH CDR1, 2 and 3 sequences set forth in SEQ ID NOs: 25, 26 and 27 respectively and VL CDR1, 2 and 3 sequences set forth in SEQ ID NOs: 28, 29 and 30 respectively;
v. VH CDR1, 2 and 3 sequences set forth in SEQ ID NOs: 37, 38 and 39 respectively and VL CDR1, 2 and 3 sequences set forth in SEQ ID NOs: 40, 41 and 42 respectively;
vi. VH CDR1, 2 and 3 sequences set forth in SEQ ID NOs: 1, 2 and 3 respectively and VL CDR1, 2 and 3 sequences set forth in SEQ ID NOs: 4, 5 and 6 respectively;
vii. VH CDR1, 2 and 3 sequences set forth in SEQ ID NOs: 13, 14 and 15 respectively and VL CDR1, 2 and 3 sequences set forth in SEQ ID NOs: 16, 17 and 18 respectively;
viii. VH CDR1, 2 and 3 sequences set forth in SEQ ID NOs: 31, 32 and 33 respectively and VL CDR1, 2 and 3 sequences set forth in SEQ ID NOs: 34, 35 and 36 respectively;
ix. VH CDR1, 2 and 3 sequences set forth in SEQ ID NOs: 43, 44 and 45 respectively and VL CDR1, 2 and 3 sequences set forth in SEQ ID NOs: 46, 47 and 48 respectively;
x. VH CDR1, 2 and 3 sequences set forth in SEQ ID NOs: 49, 50 and 51 respectively and VL CDR1, 2 and 3 sequences set forth in SEQ ID NOs: 52, 53 and 54 respectively;
xi. VH CDR1, 2 and 3 sequences set forth in SEQ ID NOs: 55, 56 and 57 respectively and VL CDR1, 2 and 3 sequences set forth in SEQ ID NOs: 58, 59 and 60 respectively;
xii. VH CDR1, 2 and 3 sequences set forth in SEQ ID NOs: 61, 62 and 63 respectively and VL CDR1, 2 and 3 sequences set forth in SEQ ID NOs: 64, 65 and 66 respectively;
xiii. VH CDR1, 2 and 3 sequences set forth in SEQ ID NOs: 67, 68 and 69 respectively and VL CDR1, 2 and 3 sequences set forth in SEQ ID NOs: 70, 71 and 72 respectively; or
xiv. VH CDR1, 2 and 3 sequences set forth in SEQ ID NOs: 79, 80 and 81 respectively and VL CDR1, 2 and 3 sequences set forth in SEQ ID NOs: 82, 83 and 84 respectively;
   wherein one or more of said CDR sequences of (i) to (xiv) may optionally comprise 1 to 3 amino acid modifications relative to an aforementioned CDR sequence, particularly wherein one or more of said CDR sequences may optionally be modified by substitution, addition or deletion of 1 to 3 amino acids.

In certain embodiments, the antigen recognition domain comprises:
i. a VH domain comprising the sequence set forth in SEQ ID NO: 87, or a sequence having at least 70% sequence identity thereto, and a VL domain comprising the sequence as set forth in SEQ ID NO: 88, or a sequence having at least 70% identity thereto;
ii. a VH domain comprising the sequence set forth in SEQ ID NO: 91, or a sequence having at least 70% sequence identity thereto, and a VL domain comprising the sequence as set forth in SEQ ID NO: 92, or a sequence having at least 70% identity thereto;
iii. a VH domain comprising the sequence set forth in SEQ ID NO: 109, or a sequence having at least 70% sequence identity thereto, and a VL domain comprising the sequence as set forth in SEQ ID NO: 110, or a sequence having at least 70% identity thereto;
iv. a VH domain comprising the sequence set forth in SEQ ID NO: 93, or a sequence having at least 70% sequence identity thereto, and a VL domain comprising the sequence as set forth in SEQ ID NO: 94, or a sequence having at least 70% identity thereto;
v. a VH domain comprising the sequence set forth in SEQ ID NO: 97, or a sequence having at least 70% sequence identity thereto, and a VL domain comprising the sequence as set forth in SEQ ID NO: 98, or a sequence having at least 70% identity thereto;
vi. a VH domain comprising the sequence set forth in SEQ ID NO: 85, or a sequence having at least 70% sequence identity thereto, and a VL domain comprising the sequence as set forth in SEQ ID NO: 86, or a sequence having at least 70% identity thereto;
vii. a VH domain comprising the sequence set forth in SEQ ID NO: 89, or a sequence having at least 70% sequence identity thereto, and a VL domain comprising the sequence as set forth in SEQ ID NO: 90, or a sequence having at least 70% identity thereto;
viii. a VH domain comprising the sequence set forth in SEQ ID NO: 95, or a sequence having at least 70% sequence identity thereto, and a VL domain comprising the sequence as set forth in SEQ ID NO: 96, or a sequence having at least 70% identity thereto;
ix. a VH domain comprising the sequence set forth in SEQ ID NO: 99, or a sequence having at least 70% sequence identity thereto, and a VL domain comprising the sequence as set forth in SEQ ID NO: 100, or a sequence having at least 70% identity thereto;
x. a VH domain comprising the sequence set forth in SEQ ID NO: 101, or a sequence having at least 70% sequence identity thereto, and a VL domain comprising the sequence as set forth in SEQ ID NO: 102, or a sequence having at least 70% identity thereto;
xi. a VH domain comprising the sequence set forth in SEQ ID NO: 103, or a sequence having at least 70% sequence identity thereto, and a VL domain comprising the sequence as set forth in SEQ ID NO: 104, or a sequence having at least 70% identity thereto;
xii. a VH domain comprising the sequence set forth in SEQ ID NO: 105, or a sequence having at least 70% sequence identity thereto, and a VL domain comprising the sequence as set forth in SEQ ID NO: 106, or a sequence having at least 70% identity thereto;
xiii. a VH domain comprising the sequence set forth in SEQ ID NO: 107, or a sequence having at least 70% sequence identity thereto, and a VL domain comprising the sequence as set forth in SEQ ID NO: 108, or a sequence having at least 70% identity thereto; or
xiv. a VH domain comprising the sequence set forth in SEQ ID NO: 111, or a sequence having at least 70% sequence identity thereto, and a VL domain comprising the sequence as set forth in SEQ ID NO: 112, or a sequence having at least 70% identity thereto.

In certain embodiments, the antigen recognition domain comprises or consists of:
i. the sequence set forth in SEQ ID NO: 114 or a sequence having at least 70% sequence identity thereto;
ii. the sequence set forth in SEQ ID NO: 116 or a sequence having at least 70% sequence identity thereto;
iii. the sequence set forth in SEQ ID NO: 125 or a sequence having at least 70% sequence identity thereto;
iv. the sequence set forth in SEQ ID NO: 117 or a sequence having at least 70% sequence identity thereto;
v. the sequence set forth in SEQ ID NO: 119 or a sequence having at least 70% sequence identity thereto;
vi. the sequence set forth in SEQ ID NO: 113 or a sequence having at least 70% sequence identity thereto;
vii. the sequence set forth in SEQ ID NO: 115 or a sequence having at least 70% sequence identity thereto;
viii. the sequence set forth in SEQ ID NO: 118 or a sequence having at least 70% sequence identity thereto;
ix. the sequence set forth in SEQ ID NO: 120 or a sequence having at least 70% sequence identity thereto;
x. the sequence set forth in SEQ ID NO: 121 or a sequence having at least 70% sequence identity thereto;
xi. the sequence set forth in SEQ ID NO: 122 or a sequence having at least 70% sequence identity thereto;
xii. the sequence set forth in SEQ ID NO: 123 or a sequence having at least 70% sequence identity thereto;
xiii. the sequence set forth in SEQ ID NO: 124 or a sequence having at least 70% sequence identity thereto; or
xiv. the sequence set forth in SEQ ID NO: 126 or a sequence having at least 70% sequence identity thereto.

In certain embodiments, the CAR comprises or consists of the sequence of any one of SEQ ID NOs: 127 to 152 or a functional variant having at least 70% sequence identity thereto. For example, the CAR may comprise or consist of the sequence of SEQ ID NO: 128 or 130 or a functional variant having at least 70% sequence identity thereto.

In certain embodiments, the CAR may comprise:
a. an exodomain comprising an antigen recognition domain;
b. a transmembrane domain; and
c. an endodomain comprising an intracellular signalling domain.

In certain embodiments, the CAR further comprises a hinge domain and/or one or more co-stimulatory domains. In certain embodiments, the CAR comprises a CD8α or CH2CH3 hinge domain, a CD28, CD8α or CH2CH3 transmembrane domain, a CD28 co-stimulatory domain, and a CD3zeta signalling domain, wherein when the hinge domain is CD8α, the transmembrane domain is CD8α, and when the hinge domain is CH2CH3, the transmembrane domain is CD28 or CH2CH3.

In certain embodiments, the FOXP3 polypeptide has the sequence of SEQ ID NO: 200, SEQ ID NO: 201, SEQ ID NO: 202, SEQ ID NO: 203, SEQ ID NO: 204, SEQ ID NO: 205 or a variant having at least 70% identity thereto. In certain embodiments, the second nucleotide sequence encoding the FOXP3 polypeptide has the sequence of SEQ ID NO: 206, SEQ ID NO: 207 or a sequence having at least 70% identity thereto.

In certain embodiments, the expression construct comprises, consists essentially of or consists of the sequence of SEQ ID NO: 214 or a sequence having at least 70% identity thereto.

In certain embodiments, the expression construct may comprise or consist of 5′ to 3′:
(i) a PGK promoter;
(ii) a first nucleotide sequence encoding a CAR;
(iii) a nucleotide sequence encoding a self-cleavage sequence (e.g. a 2A sequence such as P2A, T2A, E2A or F2A); and
(iv) a second nucleotide sequence encoding a FOXP3 polypeptide;
wherein the PGK promoter is operably linked to the first nucleotide sequence and second nucleotide sequence.

In certain embodiments, the expression construct may comprise or consist of 5′ to 3′:
(i) a PGK promoter;
(ii) a first nucleotide sequence encoding a CAR, wherein the CAR has VH CDR1, 2 and 3 sequences set forth in SEQ ID NOs: 7, 8 and 9 respectively and VL CDR1, 2 and 3 sequences set forth in SEQ ID NOs: 10, 11 and 12 respectively, wherein one or more of said CDR sequences may optionally comprise 1 to 3 amino acid modifications relative to an aforementioned CDR sequence;
(iii) a nucleotide sequence encoding a self-cleavage sequence (e.g. a 2A sequence such as P2A, T2A, E2A or F2A); and
(iv) a second nucleotide sequence encoding a FOXP3 polypeptide;
wherein the PGK promoter is operably linked to the first nucleotide sequence and second nucleotide sequence.

In certain embodiments, the expression construct may comprise or consist of 5′ to 3′:
(i) a PGK promoter;
(ii) a first nucleotide sequence encoding a CAR, wherein the CAR has VH CDR1, 2 and 3 sequences set forth in SEQ ID NOs: 19, 20 and 21 respectively and VL CDR1, 2 and 3 sequences set forth in SEQ ID NOs: 22, 23 and 24 respectively, wherein one or more of said CDR sequences may optionally comprise 1 to 3 amino acid modifications relative to an aforementioned CDR sequence;
(iii) a nucleotide sequence encoding a self-cleavage sequence (e.g. a 2A sequence such as P2A, T2A, E2A or F2A); and
(iv) a second nucleotide sequence encoding a FOXP3 polypeptide;
wherein the PGK promoter is operably linked to the first nucleotide sequence and second nucleotide sequence.

In certain embodiments, the expression construct may comprise or consist of 5′ to 3′:
(i) a PGK promoter comprising or consisting of the sequence of SEQ ID NO: 195 or a functional variant or functional fragment thereof, optionally having at least 70% identity thereto;
(ii) a first nucleotide sequence encoding a CAR, wherein the CAR has VH CDR1, 2 and 3 sequences set forth in SEQ ID NOs: 7, 8 and 9 respectively and VL CDR1, 2 and 3 sequences set forth in SEQ ID NOs: 10, 11 and 12 respectively, wherein one or more of said CDR sequences may optionally comprise 1 to 3 amino acid modifications relative to an aforementioned CDR sequence;
(iii) a nucleotide sequence encoding a self-cleavage sequence (e.g. a 2A sequence such as P2A, T2A, E2A or F2A); and
(iv) a second nucleotide sequence encoding a FOXP3 polypeptide comprising or consisting of the sequence of SEQ ID NO: 200 or a functional variant thereof, optionally having at least 70% identity thereto;
wherein the PGK promoter is operably linked to the first nucleotide sequence and second nucleotide sequence.

In certain embodiments, the expression construct may comprise or consist of 5′ to 3′:
(i) a PGK promoter comprising or consisting of the sequence of SEQ ID NO: 195 or a functional variant or functional fragment thereof, optionally having at least 70% identity thereto;
(ii) a first nucleotide sequence encoding a CAR, wherein the CAR has VH CDR1, 2 and 3 sequences set forth in SEQ ID NOs: 19, 20 and 21 respectively and VL CDR1, 2 and 3 sequences set forth in SEQ ID NOs: 22, 23 and 24 respectively, wherein one or more of said CDR sequences may optionally comprise 1 to 3 amino acid modifications relative to an aforementioned CDR sequence;
(iii) a nucleotide sequence encoding a self-cleavage sequence (e.g. a 2A sequence such as P2A, T2A, E2A or F2A); and
(iv) a second nucleotide sequence encoding a FOXP3 polypeptide comprising or consisting of the sequence of SEQ ID NO: 200 or a functional variant thereof, optionally having at least 70% identity thereto;
wherein the PGK promoter is operably linked to the first nucleotide sequence and second nucleotide sequence.

In certain embodiments, the expression construct may comprise or consist of 5′ to 3′:
(i) a PGK promoter comprising or consisting of the sequence of SEQ ID NO: 195 or a functional variant or functional fragment thereof, optionally having at least 70% identity thereto;
(ii) a first nucleotide sequence encoding a CAR encoding a CAR comprising or consisting of the sequence of SEQ ID NO: 128 or a functional variant or functional fragment thereof, optionally having at least 70% identity thereto;
(iii) a nucleotide sequence encoding a self-cleavage sequence (e.g. a 2A sequence such as P2A, T2A, E2A or F2A); and
(iv) a second nucleotide sequence encoding a FOXP3 polypeptide comprising or consisting of the sequence of SEQ ID NO: 200 or a functional variant thereof, optionally having at least 70% identity thereto;
wherein the PGK promoter is operably linked to the first nucleotide sequence and second nucleotide sequence.

In certain embodiments, the expression construct may comprise or consist of 5′ to 3′:
(i) a PGK promoter comprising or consisting of the sequence of SEQ ID NO: 195 or a functional variant or functional fragment thereof, optionally having at least 70% identity thereto;
(ii) a first nucleotide sequence encoding a CAR comprising or consisting of the sequence of SEQ ID NO: 130 or a functional variant or functional fragment thereof, optionally having at least 70% identity thereto;
(iii) a nucleotide sequence encoding a self-cleavage sequence (e.g. a 2A sequence such as P2A, T2A, E2A or F2A); and
(iv) a second nucleotide sequence encoding a FOXP3 polypeptide comprising or consisting of the sequence of SEQ ID NO: 200 or a functional variant thereof, optionally having at least 70% identity thereto;
wherein the PGK promoter is operably linked to the first nucleotide sequence and second nucleotide sequence.

In certain embodiments, the expression construct may comprise or consist of 5′ to 3′:
(i) a PGK promoter having the sequence of SEQ ID NO: 195;
(ii) a first nucleotide sequence encoding a CAR having the sequence of SEQ ID NO: 128;
(iii) a nucleotide sequence encoding a self-cleavage sequence (e.g. a 2A sequence such as P2A, T2A, E2A or F2A); and
(iv) a second nucleotide sequence encoding a FOXP3 polypeptide having the sequence of SEQ ID NO: 200;
wherein the PGK promoter is operably linked to the first nucleotide sequence and second nucleotide sequence.

In certain embodiments, the expression construct may comprise or consist of 5′ to 3′:
(i) a PGK promoter having the sequence of SEQ ID NO: 195;
(ii) a first nucleotide sequence encoding a CAR having the sequence of SEQ ID NO: 130;
(iii) a nucleotide sequence encoding a self-cleavage sequence (e.g. a 2A sequence such as P2A, T2A, E2A or F2A); and
(iv) a second nucleotide sequence encoding a FOXP3 polypeptide having the sequence of SEQ ID NO: 200;
wherein the PGK promoter is operably linked to the first nucleotide sequence and second nucleotide sequence.

In a second aspect, the invention provides a nucleic acid molecule comprising the expression construct of the first aspect of the invention, including any embodiment thereof.

In certain embodiments, the nucleic acid molecule does not comprise any other coding nucleotide sequence. Alternatively put, the nucleic acid molecule does not comprise any other coding nucleotide sequence operably linked to the same PGK promoter present in the expression construct and does not comprise any other coding nucleotide sequence operably linked to a different promoter. In other words, the nucleic acid molecule defined herein may be bicistronic because it encodes two discrete proteins (the CAR and the FOXP3 polypeptide encoded by the first and second nucleotide sequences in the expression construct). By “coding sequence” or “coding region” it is meant the nucleotide sequence that is translated and thus codes for a discrete protein.

In a third aspect, the invention provides a vector comprising the expression construct of the first aspect of the invention and/or the nucleic acid molecule of the second aspect of the invention, including any embodiment thereof. The vector may be a plasmid or a viral vector. For example, the vector may be a lentiviral vector or a gamma-retroviral vector.

In a fourth aspect, the invention provides a cell comprising the expression construct of the first aspect of the invention and/or the nucleic acid molecule of the second aspect of the invention and/or the vector of the third aspect of the invention, including any embodiment thereof.

The cell is thus an engineered cell, or alternatively viewed, a cell which has been modified to introduce the expression construct or the nucleic acid molecule or vector as defined herein. The cell recombinantly expresses the expression construct or the nucleic acid molecule. The cell may be a cell for production of the polypeptide, or for production of the expression construct, nucleic acid molecule or vector, for example for producing the vector. Accordingly, the cell may be a production host cell. Alternatively, the cell may be a cell intended for use in therapy, particularly ACT. Thus, the cell may be an immune cell or a progenitor or precursor thereof. Optionally, the cell may be a T cell, or a precursor thereof, or a stem cell. In particular, the cell may be a Treg, or a precursor thereof, or an iPSC cell. In a specific embodiment, the invention provides a Treg comprising the expression construct, nucleic acid molecule and/or vector as defined herein. The Treg expresses the CAR and FOXP3 polypeptide encoded by the first and second nucleotide sequences in the expression construct.

The cell may be provided in a cell population, which forms a further aspect of the invention. In particular, the cell population may comprise a plurality of cells according to the invention, particularly a plurality of T cells (e.g. a plurality of Tregs) according to the invention.

The invention also provides a pharmaceutical composition comprising the cell, cell population or vector according to the invention.

In another aspect, the invention provides a cell, cell population or pharmaceutical composition according to the invention for use in therapy (e.g., for use in induction of tolerance to a transplant, treating and/or preventing graft-versus-host disease (GvHD), treating and/or preventing an infectious, allergic, autoimmune or inflammatory disease, or for use in inducing immunosuppression, or for use in promoting tissue repair and/or tissue regeneration). The therapy may be adoptive cell transfer therapy.

Alternatively viewed, the invention provides a method for inducing tolerance to a transplant, treating and/or preventing graft-versus-host disease (GvHD), treating and/or preventing an infectious, allergic, autoimmune or inflammatory disease, or for inducing immunosuppression, or for promoting tissue repair and/or tissue regeneration, wherein the method comprises administering a cell to a subject, particularly a Treg cell, a cell population, or a pharmaceutical composition, particularly comprising a Treg, according to the invention.

In this respect, the method may comprise the following steps:
(i) isolation or provision of a Treg-enriched cell sample from a subject;
(ii) introduction into the Treg cells of an expression construct, nucleic acid molecule or vector of the invention; and
(iii) administering the Treg cells from (ii) to the subject.

The invention also provides use of a cell, cell population or pharmaceutical composition according to the invention in the manufacture of a medicament for inducing tolerance to a transplant, treating and/or preventing graft-versus-host disease (GvHD), treating and/or preventing an infectious, allergic, autoimmune or inflammatory disease, or for inducing immunosuppression, or for promoting tissue repair and/or tissue regeneration in a subject, particularly wherein the cell is a Treg cell.

The autoimmune or inflammatory disease may particularly be type 1 diabetes (T1D).

In another aspect, the invention provides a method of making a cell according to the invention, which comprises the step of introducing into the cell (e.g., transducing or transfecting a cell with) the expression construct, nucleic acid molecule or vector according to the invention. The cell may be a Treg cell, and the method may comprise isolating or providing a cell-containing sample comprising Tregs, and/or enriching Tregs or generating Tregs from the cell-containing sample prior to or after the step of introducing the expression construct, nucleic acid molecule or vector into the cell. The invention also provides a cell obtainable by this method, which forms a further aspect of the invention.

### BRIEF DESCRIPTION OF THE FIGURES

FIG. 1 shows the expression constructs tested in Examples 1, 2, 3 and 5.

FIG. 2 shows the % of Tregs expressing the CAR (top plot) and MFI of the CAR in CAR+ cells (bottom plot) following transduction with either the SFFV-CAR-FOXP3 construct or PGK-CAR-FOXP3 construct.

FIG. 3 shows the % of Tregs expressing FOXP3 (top plot) and MFI of FOXP3 in CD4+ cells (bottom plot) following transduction with either the SFFV-CAR-FOXP3 construct or PGK-CAR-FOXP3 construct.

FIG. 4 shows % of transduced (CAR+) Tregs expressing the activation marker CD69 following transduction with either the SFFV-CAR-FOXP3 construct or PGK-CAR-FOXP3 construct and exposure to antigen (ENTPD3+HEK, RT4 cells at 10:1, 5:1 and 1:1 and 5, 2, 1 or 0.5 ug/mL of ENTPD3 peptide). As a negative control, cells were exposed to media only (unstimulated) or WT HEK cells. As a positive control, cells were exposed to anti-CD3/CD28 beads.

FIG. 5 shows Treg proliferation (indicated by fold change) following transduction with either the SFFV-CAR-FOXP3 construct or PGK-CAR-FOXP3 construct and exposure to antigen (ENTPD3+HEK, RT4 cells at 10:1, 5:1 or 1:1 and 5, 2 or 1 ug/mL of ENTPD3 peptide). As a negative control, cells were exposed to media only (unstimulated) or WT HEK cells. As a positive control, cells were exposed to anti-CD3/CD28 beads.

FIG. 6 shows Teff suppression by Tregs transduced with either the SFFV-CAR-FOXP3 construct (top plot) or PGK-CAR-FOXP3 construct (middle plot) or untransduced (mock) Tregs (bottom plot) following activation by CD3/CD28 beads and/or B cells expressing antigen. As a negative control, cells were exposed to media only (unstimulated) or exposed to WT (ENTPD3−) B cells.

FIG. 7 shows IL-2 production by Teffs transduced with either the SFFV-CAR-FOXP3 construct or PGK-CAR-FOXP3 construct or by a construct comprising only SFFV-CAR without FOXP3. The flow cytometry results are shown in the top plot and the supernatant analysis is shown in the bottom plot.

FIG. 8 shows expansion fold of cells transduced with the PGK-CAR-FOXP3 construct and cells transduced with the SFFV-CAR-FOXP3 construct across the different donors.

FIG. 9 shows the phenotype analysis of cells transduced with the PGK-CAR-FOXP3 construct and cells transduced with the SFFV-CAR-FOXP3 construct.

FIG. 10 shows % of transduced (CAR+) Tregs expressing the activation markers CD69, CD137 and GARP following transduction with either the SFFV-CAR-FOXP3 construct or PGK-CAR-FOXP3 construct and exposure to antigen (ENTPD3+HEK cells or EndoC-βH5 cells). As a negative control, cells were exposed to media only (no stim) or WT HEK cells. As a positive control, cells were exposed to anti-CD3/CD28 beads.

FIG. 11 shows CD4 and CD8 suppression by Tregs transduced with PGK-CAR-FOXP3 construct (left hand plots) or SFFV-CAR-FOXP3 construct (middle plots) or untransduced (mock) Tregs (right hand plots) following activation by CD3/CD28 beads and/or B cells expressing antigen. As a negative control, cells were exposed to WT (ENTPD3−) B cells.

FIG. 12 shows the difference in area under the under (ΔAUC) for CD4 and CD8 suppression shown in FIG. 10 (ΔAUC=% suppression when exposed to ENTPD3+ B cells minus % suppression when exposed to WT B cells).

FIG. 13 shows depletion of CD80 and CD86 following Treg exposure to either WT B cells or B cells expressing ENTPD3. CD80 and CD86 expression in the Treg conditions were normalized to the B cell alone control. % suppression of CD80 and CD86 was plotted and area under the curve calculated.

FIG. 14 shows the expression constructs tested in Example 4.

FIG. 15 shows % of transduced (CAR+) Tregs expressing the activation markers CD69, CD137 and GARP following transduction with the constructs shown in FIG. 14 and exposure to antigen (peptide of antigen-expressing HEK293 cells). As a negative control, cells were exposed to media only (no stim) or WT HEK cells. As a positive control, cells were exposed to anti-CD3/CD28 beads.

FIG. 16 shows Treg proliferation (indicated by fold change) following transduction with the constructs shown in FIG. 14 and exposure to antigen-expressing B cells. As a negative control, cells were exposed to media only (no stim) or WT B cells. As a positive control, cells were exposed to anti-CD3/CD28 beads.

FIG. 17 shows Teff suppression by Tregs transduced with either the constructs shown in FIG. 14 following activation by CD3/CD28 beads or B cells expressing antigen. As a negative control, cells were exposed to exposed to WT B cells.

FIG. 18 shows fold change of CAR+ cells following the survival assay performed in Example 4.

FIG. 19 shows representative immunofluorescence images of human islet microtissues (hIsMTs) stained for DAPI, NKX6.1, and insulin following 3 days of treatment. Conditions include exposure to a pro-inflammatory cytokine cocktail (CC), co-culture with effector T cells (Teff) and irradiated B cells, or co-culture with Teff cells, irradiated B cells, and Treg cells (1:16-1:2 ratios). All images were acquired using identical exposure settings to enable direct comparison. Scale bar, 50 μm.

FIG. 20 shows NKX6.1 and Insulin expression in hIsMTs. Data represent mean±SEM from one donor, with n=6-11 technical replicates per condition. Outliers were identified and excluded using the ROUT test (Q=5%). Statistical analysis was performed using one-way ANOVA followed by Dunnett's multiple comparisons test, comparing each Treg condition to its corresponding Teff control (denoted by black asterisks). Unpaired t-tests were used to compare the hIsMT group with (1) hIsMT+Cytokine Cocktail (CC) group and (2) the Teff control condition (denoted by red asterisks). *p<0.05, **p<0.01, ***p<0.001.

FIG. 21 shows the expression constructs tested in Example 1A and 1B.

FIG. 22 shows % expression of the CAR, CACR and FOXP3 transgenes in SupT1 cells at various vector dilutions.

FIG. 23 shows % expression of the CAR, CACR and FOXP3 transgenes in SupT1 cells at no vector dilution.

### DETAILED DESCRIPTION

The present invention provides an expression construct which encodes polypeptides useful for expression in cells for ACT, specifically in the context of modifying a cell to express a CAR and a FOXP3 polypeptide. The expression constructs are particularly suitable for modifying T cells, particularly Tregs.

The present invention provides an expression comprising a PGK promoter operably linked to:
(i) a first nucleotide sequence encoding a chimeric antigen receptor (CAR); and
(ii) a second nucleotide sequence encoding a FOXP3 polypeptide;
   wherein the first nucleotide sequence is located upstream of the second nucleotide sequence.

A nucleotide sequence that is closer to the 5′ end of a nucleic acid as compared to a second nucleotide sequence is referred to as “upstream”. Therefore, in other words, the present invention provides an expression construct comprising 5′ to 3′:
a PGK promoter operably linked to;
   (i) a first nucleotide sequence encoding a chimeric antigen receptor (CAR); and
   (ii) a second nucleotide sequence encoding a FOXP3 polypeptide.

The first and second nucleotide sequences are operably linked to the same PGK promoter. By “operably linked to the same promoter” it is meant that there is a functional linkage between the PGK promoter and both the first and second nucleotide sequence that results in expression of the first and second nucleotide sequences. In other words, transcription of the first and second nucleotide sequences is initiated from the same PGK promoter and that the nucleotide sequences are positioned and oriented for transcription to be initiated from the promoter. Polynucleotides operably linked to a promoter are under transcriptional regulation of that promoter. The expression construct thus enables expression of the CAR and FOXP3 polypeptide. As used herein, the term “expression construct” refers to a nucleotide sequence in which the coding sequences are operably linked/under the control of the same promoter.

The expression construct described herein may be comprised within a nucleic acid molecule. In other words, there is also provided herein a nucleic acid molecule comprising an expression construct as described herein. The term “expression construct” refers to the PGK promoter and any nucleotide sequences operably linked to/under the control of the PGK promoter (e.g. the nucleic acid sequence starting with the PGK promoter and ending with the sequence encoding the FOXP3 polypeptide).

Therefore, there is also provided herein a nucleic acid molecule comprising a PGK promoter operably linked to:
(i) a first nucleotide sequence encoding a chimeric antigen receptor (CAR); and
(ii) a second nucleotide sequence encoding a FOXP3 polypeptide;
   wherein the first nucleotide sequence is located upstream of the second nucleotide sequence.

Alternatively viewed, there is also provided herein a nucleic acid molecule comprising 5′ to 3′:
a PGK promoter operably linked to;
   (i) a first nucleotide sequence encoding a chimeric antigen receptor (CAR); and
   (ii) a second nucleotide sequence encoding a FOXP3 polypeptide.

In certain embodiments, the expression construct does not comprise any other coding nucleotide sequence or, alternatively put, does not comprise any other coding nucleotide sequence operably linked to the same PGK promoter. In other words, the expression construct defined herein may be bicistronic because it encodes two discrete proteins (the CAR and the FOXP3 polypeptide encoded by the first and second nucleotide sequences). By “coding sequence” or “coding region” it is meant the nucleotide sequence that is translated and thus codes for a discrete protein.

In certain embodiments, the nucleic acid molecule does not comprise any other coding nucleotide sequence. Alternatively put, the nucleic acid molecule does not comprise any other coding nucleotide sequence operably linked to the same PGK promoter present in the expression construct and does not comprise any other coding nucleotide sequence operably linked to a different promoter. In other words, the nucleic acid molecule defined herein may be bicistronic because it encodes two discrete proteins (the CAR and the FOXP3 polypeptide encoded by the first and second nucleotide sequences in the expression construct). By “coding sequence” or “coding region” it is meant the nucleotide sequence that is translated and thus codes for a discrete protein.

In certain embodiments, the nucleic acid molecule or expression construct may comprise one or more further coding nucleotide sequences. For example, the nucleic acid molecule or expression construct may comprise three or more coding sequence (including the first and second nucleotide sequences encoding the CAR and FOXP3 polypeptide). For example, the nucleic acid molecule or expression construct may be tricistronic (encodes three discrete proteins).

Thus, in certain embodiments the expression construct may comprise one or more further coding nucleotide sequences operably linked to the PGK promoter. In certain embodiments, the nucleic acid molecule may comprise one or more further coding nucleotide sequences, which may be operably linked to one or more further promoters (i.e. in addition to the PGK promoter in the expression construct of the invention). The nucleic acid molecule may therefore comprise an expression construct of the invention and at least a second promoter operably linked to one or more coding sequences. The one or more further promoters may be any promoter capable of initiating transcription of the one or more further coding nucleotide sequences, e.g., a mammalian promoter such as PGK or EF1α (short or long versions). Where the nucleic acid molecule comprises one or more further coding nucleotide sequences, the expression construct may or may not comprise any further coding sequences.

The polypeptides may be produced in a cell as individual components, i.e., as discrete entities. Thus, the CAR and FOXP3 polypeptides may be separately located, in or on the cell, as separate and distinct, or discrete, functional polypeptides. Thus, although the polypeptides are encoded by a single nucleic acid molecule or expression construct, through “cleavage” during or after translation, they may be expressed or produced as separate polypeptides, and thus at the end of the protein production process in the cell, they may be present in the cell as separate entities, or separate polypeptide chains. By “discrete” or “separate” polypeptides, it is meant that the polypeptides are not linked to one another and are physically distinct. In other words they are expressed or produced as separate entities. Indeed, following expression, they are located in different, or separate cellular locations. The CAR and FOXP3 polypeptide are thus ultimately expressed as single and separate components. The CAR is expressed as a cell surface molecule. The FOXP3 is expressed inside the cell, where it can exert its effect as a transcription factor to regulate cell development and/or activity, as described further below.

This may be achieved by the presence of or by encoding cleavage sequences in the expression construct, in particular self-cleaving sequences, in between the nucleotide sequences encoding the discrete polypeptides, particularly between the first nucleotide sequence and second nucleotide sequence of the expression construct of the invention. For example, 2A or 2A-like peptides may be used as the self-cleaving sequences. Although described as “self-cleaving”, such peptides are believed to function by allowing ribosome skipping such that a peptide bond is not formed (skipped) at the C-terminus of the 2A sequence, leading to separation of the 2A sequence and the next polypeptide downstream of it. The term “cleavage” as used herein thus includes the skipping of peptide bond formation.

2A and 2A-like peptides are known and described in the art, for example in Donnelly et al., Journal of General Virology, 2001, 82, 1027-1041, herein incorporated by reference. As noted above, 2A and 2A-like peptides are believed to cause ribosome skipping, and result in a form of cleavage in which a ribosome skips the formation of a peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline. For avoidance of doubt, the self-cleaving sequences such as the 2A sequences disclosed herein are not considered to be discrete proteins encoded by the expression construct or nucleic acid molecule. Constructs comprising only a first nucleotide sequence encoding a CAR separated from a second nucleotide sequence encoding a FOXP3 polypeptide by a 2A sequence is thus bicistronic.

Suitable self-cleaving domains include P2A, T2A, E2A and F2A sequences as shown in SEQ ID NOs: 208 to 211 respectively. The sequences may be modified to include the amino acids GSG at the N-terminus of the 2A peptides. Thus, also included as possible options are the sequences corresponding to SEQ ID NOs: 208 to 211, but with GSG at the N termini thereof. Such modified alternative 2A sequences are known and reported in the art. Alternative 2A-like sequences which may be used are shown in Donnelly et al (supra), for example a TaV sequence. In certain embodiments, a nucleotide sequence encoding the T2A self-cleaving sequence is positioned between the first and second nucleotide sequences in the expression construct described herein.

The self-cleaving sequence may include an additional cleavage site, which may be cleaved by common enzymes present in the cell. This may assist in achieving complete removal of the 2A sequences after translation. Such an additional cleavage site may for example comprising a Furin cleavage site RXXR (SEQ ID NO: 212), for example RRKR (SEQ ID NO: 213)

Alternatively or additionally, an internal ribosome entry site (IRES) may be present between or encoded by a nucleotide sequence positioned between the first nucleotide sequence and second nucleotide sequence.

Where the expression construct includes three or more coding sequences (three or more sequences encoding and resulting in translation of discrete proteins), a self-cleaving sequence may be encoded between each coding sequence (e.g. between the first and second nucleotide sequence and between the second and third coding sequence). The self-cleaving sequences may be the same or different. For example, they may both be 2A or 2A-like sequence, in particular P2A and/or T2A sequences.

In an embodiment, the expression construct may comprise or consist of 5′ to 3′:
(i) a PGK promoter;
(ii) a first nucleotide sequence encoding a CAR;
(iii) a nucleotide sequence encoding a self-cleavage sequence (e.g. a 2A sequence such as P2A, T2A, E2A or F2A); and
(iv) a second nucleotide sequence encoding a FOXP3 polypeptide;
wherein the PGK promoter is operably linked to the first nucleotide sequence and second nucleotide sequence. The PGK promoter, CAR and FOXP3 polypeptide may comprise, consist essentially of or consist of any of the sequences described herein or functional variants or functional fragments thereof, for example having at least 70%, 80%, 90%, 95% or 98% identity thereto.

Nucleic acid molecules and polynucleotides/nucleotides/nucleic acid sequences as defined herein may comprise DNA or RNA. They may be single-stranded or double-stranded. It will be understood by a skilled person that numerous different nucleic acid molecules/polynucleotides can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides/nucleotide sequences as defined herein to reflect the codon usage of any particular host organism in which the polypeptides of the invention are to be expressed.

The nucleic acid molecules/polynucleotides/nucleotides may be modified by any method available in the art. Such modifications may be carried out in order to enhance the in vivo activity or lifespan of the nucleic acid molecules/polynucleotides as defined herein.

Expression constructs/nucleic acid molecules/polynucleotides/nucleotide sequences such as DNA nucleic acid molecules/polynucleotides/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The expression construct/nucleic acid molecule/polynucleotides/nucleotides used in the present invention may be codon-optimised. Codon optimisation has previously been described in WO1999/41397 and WO2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

As is clear from the above description, in addition to the specific polypeptide and nucleotide sequences mentioned herein, also encompassed is the use of variants, or derivatives and fragments thereof.

In certain embodiments, the expression construct comprises, consists essentially of or consists of the sequence of SEQ ID NO: 214 or a sequence having at least 70% identity thereto. For example, the expression construct may comprise, consist essentially of or consist of the sequence of SEQ ID NO: 214 or a sequence having at least 75%, 80%, 85%, 90%, 95%, 96%, 97%, 98% or 99% identity thereto. The sequence having at least 70% identity thereto (e.g. at least 75, 80, 85, 90, 95, 96, 97. 98 or 99% identity thereto) is a functional variant in that it is able to drive the expression of a functional CAR and functional FOXP3 polypeptide as described herein.

The term “derived from” as used herein, in relation to nucleotide or polypeptide sequences means that the sequence is the same as the reference nucleotide or polypeptide sequence or includes one or more variations (e.g. substitutions, insertions, deletions, truncations) compared to the reference nucleotide or polypeptide sequence, provided that the resultant nucleotide or polypeptide sequence retains the desired function as described below. The reference nucleotide or polypeptide sequence may be a naturally-occurring, endogenous or wildtype sequence.

The term “derivative” or “variant” as used interchangeably herein, in relation to promoters, proteins or polypeptides or nucleic acids of the present invention includes any substitution of, variation of, modification of, replacement of, deletion of and/or addition of one (or more) amino acid residues or nucleotides from or to the sequence providing that the resultant protein or polypeptide or nucleic acid sequence retains the desired function (for example, where the derivative or variant is an antigen binding domain, the desired function may be the ability of the antigen binding domain to bind its target antigen (for example, a variant of an antigen binding domain which binds to ENTPD3 retains the ability to bind ENTPD3), where the derivative or variant is a signalling domain, the desired function may be the ability of that domain to signal (e.g. activate or inactivate a downstream molecule), where the derivative or variant is a transcription factor (e.g. FOXP3), the desired function may be the ability of the transcription factor to bind to target DNA and/or to induce transcription, or where the derivative or variant is a PGK promoter, the desired function may be the ability to initiate transcription or the expression level obtained (e.g. mRNA or protein) of the genes to which it is operably linked. Alternatively viewed, the variants or derivatives referred to herein are functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g., increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Nucleic acid molecules may also have deletions, insertions or substitutions of nucleotides. Where the nucleotide sequence is a coding sequence, deletions, insertion or substitutions of nucleotides may result in a silent change in that the amino acid encoded by the nucleotide sequence does not change due to the degeneracy of the genetic code. Coding and non-coding regions of a nucleic acid molecule may have deletion, insertion or substitution of nucleotides compared to a reference sequence. The variant or derivative nucleic acid molecule may retain at least 10%, at least 20%, at least 30%, at least 40%, at least 50%, at least 60%, at least 70%, at least 80%, or at least 90% of its function compared to the corresponding, reference sequence. The variant or derivative may have a similar or the same level of function as compared to the corresponding, reference sequence or may have an increased level of function (e.g., increased by at least 10%, at least 20%, at least 30%, at least 40% or at least 50%). For example, the function of a promoter sequence may be to initiate transcription and result in expression of transgene so transduction efficiency (e.g. % transduced cells or MFI) may be used to determine the level of function of a variant promoter sequence.

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid or nucleotide sequence which may be at least 70%, 75%, 85% or 90% identical, optionally at least 95%, 96%, 97%, 98% or 99% identical to the subject sequence. Typically, the variants will comprise the same active sites etc. as the subject sequence. Although homology can also be considered in terms of similarity (e.g., amino acid residues having similar chemical properties/functions), in the context herein homology may be expressed in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, the default values may be used when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, the GCG Bestfit program may be used. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, the public default values for the GCG package may be used, or in the case of other software, the default matrix, such as BLOSUM62. Suitably, the percentage identity is determined across the entirety of the reference and/or the query sequence.

Once the software has produced an optimal alignment, it is possible to calculate percentage homology, optionally percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### PGK Promoter

A “promoter” is a region of DNA where or which defines where transcription of a coding nucleotide sequence or gene is initiated. Promoters are typically located immediately upstream of (or 5′ to) a coding nucleotide sequence or gene position and may control expression of the nucleotide sequence or gene, e.g. by providing a site for the binding of proteins necessary for transcription.

The present disclosure provides nucleic acid molecules and/or expression constructs comprising a PGK promoter operably linked to a first nucleotide sequence encoding a CAR and a second nucleotide sequence encoding a FOXP3 polypeptide.

By “operably linked to the same promoter” it is meant that there is a functional linkage between the PGK promoter and both the first and second nucleotide sequence that results in expression of the first and second nucleotide sequences. In other words, transcription of the first and second nucleotide sequences is initiated from the same PGK promoter and that the nucleotide sequences are positioned and oriented for transcription to be initiated from the promoter. Polynucleotides operably linked to a promoter are under transcriptional regulation of that promoter.

The expression constructs and nucleic acid molecules described herein comprise a phosphoglycerate kinase 1 (PGK) promoter. As used herein, the term “PGK promoter” refers to a promoter that is or is derived from an endogenous promoter operably linked to an endogenous PGK gene in an unmodified or non-engineered cell. Alternatively viewed, the PGK promoter as used herein is derived from an endogenous, wildtype or naturally occurring PGK promoter. An endogenous, wildtype or naturally occurring PGK promoter is capable initiating transcription of an endogenous, naturally occurring or wildtype PGK gene. The PGK gene may be the human PGK gene. The PGK promoter as referred to herein may comprise, consist essentially of or consist of an identical sequence to an endogenous, wildtype or naturally occurring PGK promoter or may be a functional variant or functional fragment thereof.

By “functional variant” it is meant any variant that is able to function as a promoter (i.e., initiate transcription and result in expression of the transgene(s) to which it is operably linked). Variants therefore include PGK promoters that include one or more nucleotide truncations, deletions, substitutions or insertions. The variant may retain at least 10%, at least 20%, at least 30%, at least 40%, at least 50%, at least 60%, at least 70%, at least 80%, or at least 90% of its function compared to the corresponding, reference sequence (e.g. an endogenous, wildtype or naturally occurring PGK promoter or SEQ ID NO: 195 or nucleotides 1 to 531 of SEQ ID NO: 274). The variant or derivative may have a similar or the same level of function as compared to the corresponding, reference sequence (an endogenous, wildtype or naturally occurring PGK promoter or SEQ ID NO: 195 or nucleotides 1 to 531 of SEQ ID NO: 274) or may have an increased level of function (e.g., increased by at least 10%, at least 20%, at least 30%, at least 40% or at least 50%). For example, the function of a promoter sequence may be to initiate transcription and result in expression of transgene so transduction efficiency (e.g. % transduced cells or MFI) may be used to determine the level of function of a variant promoter sequence.

By “functional fragment” it is meant a fragment of a reference PGK promoter (e.g. a fragment of an endogenous, wildtype or naturally occurring PGK promoter or a fragment of SEQ ID NO: 195 or a fragment of nucleotides 1 to 531 of SEQ ID NO: 274) which is able to function as a promoter (i.e. initiate transcription and result in expression of the transgene to which it is operably linked). A functional fragment may further comprise one or more nucleotide deletions, substitutions or insertions.

The functional variant may, for example, have at least about 70% sequence identity to an endogenous, wildtype or naturally occurring PGK promoter. For example, the functional variant may have at least about 75% or at least about 80% or at least about 85% or at least about 90% or at least about 91% or at least about 92% or at least about 93% or at least about 94% or at least about 95% or at least about 96% or at least about 97% or at least about 98% or at least about 99% sequence identity to an endogenous, wildtype or naturally occurring PGK promoter.

A functional fragment may have at least 20% sequence identity to an endogenous, wildtype or naturally occurring PGK promoter. For example, the functional fragment may have at least about 30% or at least about 40%, or at least about 50%, or at least about 60%, or at least about 70%, or at least about 80%, or at least about 85%, or at least about 90%, or at least about 95%, or at least about 96%, or at least about 97%, or at least about 98%, or at least about 99% sequence identity to an endogenous, wildtype or naturally occurring PGK promoter. As discussed above, a functional fragment may further comprise one or more nucleotide deletions, substitutions or insertions, as compared to an endogenous, wildtype or naturally occurring PGK promoter, however, the nucleotide sequence of the fragment should have at least 70% (e.g. at least 75, 80, 90, 95, 96, 97, 98 or 99%) sequence identity to its corresponding endogenous, wildtype or naturally occurring PGK promoter fragment.

The sequence of human phosphoglycerate kinase 1 (PGK) gene is disclosed as GenBank accession no. AH002937.2 (SEQ ID NO: 274). The coding sequence (CDS) is highlighted in SEQ ID NO: 274 and starts at nucleotide 532.

The PGK promoter may therefore comprise, consist essentially of or consist of the sequence of nucleotides 1 to 531 of SEQ ID NO: 274 or a functional variant or a functional fragment thereof. The functional variant thereof may, for example, have at least about 70% sequence identity to nucleotides 1 to 531 of SEQ ID NO: 274. For example, the functional variant may have at least about 75% or at least about 80% or at least about 85% or at least about 90% or at least about 95% or at least about 96% or at least about 97% or at least about 98% or at least about 99% sequence identity to nucleotides 1 to 531 of SEQ ID NO: 274. The functional fragment thereof may, for example, have at least about 20% or at least about 30% or at least about 40%, or at least about 50%, or at least about 60%, or at least about 70%, or at least about 80%, or at least about 85%, or at least about 90%, or at least about 95%, or at least about 96%, or at least about 97%, or at least about 98%, or at least about 99% sequence identity to nucleotides 1 to 531 of SEQ ID NO: 274. As discussed above, a functional fragment may further comprise one or more nucleotide deletions, substitutions or insertions, as compared to nucleotides 1 to 531 of SEQ ID NO: 274, however, the nucleotide sequence of the fragment should have at least 70% (e.g. at least 75, 80, 90, 95, 96, 97, 98 or 99%) sequence identity to its corresponding fragment of SEQ ID NO: 274.

The PGK promoter may, for example, comprise, consist essentially of or consist of the sequence of SEQ ID NO: 195 or a functional variant or functional fragment thereof. The functional variant may, for example, have at least about 70% sequence identity to SEQ ID NO: 195. For example, the functional variant may have at least about 75% or at least about 80% or at least about 85% or at least about 90% or at least about 95% or at least about 96% or at least about 97% or at least about 98% or at least about 99% sequence identity to SEQ ID NO: 195.

A functional fragment may have at least 20% sequence identity to SEQ ID NO: 195. For example, the functional fragment may have at least about 30% or at least about 40%, or at least about 50%, or at least about 60%, or at least about 70%, or at least about 80%, or at least about 85%, or at least about 90%, or at least about 95%, or at least about 96%, or at least about 97%, or at least about 98%, or at least about 99% sequence identity to SEQ ID NO: 195. As discussed above, a functional fragment may further comprise one or more nucleotide deletions, substitutions or insertions, as compared to SEQ ID NO: 195, however, the nucleotide sequence of the fragment should have at least 70% (e.g. at least 75, 80, 90, 95, 96, 97, 98 or 99%) sequence identity to its corresponding fragment of SEQ ID NO: 195.

Alternatively viewed, a functional fragment may be referred to as a truncated PGK promoter. In certain embodiments, the PGK promoter comprises, consists essentially of or consists of a truncated PGK promoter or a functional variant thereof. In certain embodiments, the PGK promoter is a truncated human PGK promoter or a functional variant thereof. By “truncated” it is meant that a portion at the 3′ and/or 5′ end of the PGK promoter has been removed (deleted) such that the nucleotide sequence of the truncated PGK promoter is shorter than the reference PGK promoter. The reference PGK promoter may, for example, be an endogenous, wildtype or naturally occurring PGK promoter, nucleotides 1 to 531 of SEQ ID NO: 274 and/or the sequence of SEQ ID NO: 195.

Exemplary truncated PGK promoters are disclosed in WO 2016/115482, the contents of which are incorporated herein by reference.

In certain embodiments, at least about 10, 20, 30, 40, 50, 60, 70, 80, 90, 100, 150, 200, 250, 300, 350, 400 nucleotides are removed from the 5′ end of an endogenous, wildtype or naturally occurring PGK promoter or from the 5′ end of SEQ ID NO: 274 or from the 5′ end of SEQ ID NO: 195. For example, from about 5 to about 450 or from about 10 to about 450 or from about 50 to about 450 or from about 100 to about 450 nucleotides may be removed from the 5′ end of an endogenous, wildtype or naturally occurring PGK promoter or from the 5′ end of SEQ ID NO: 274 or from the 5′ end of SEQ ID NO: 195. For example, from about 5 to about 400 or from about 10 to about 350 or from about 50 to about 300 or from about 100 to about 250 nucleotides are removed from the 5′ end of an endogenous, wildtype or naturally occurring PGK promoter or from the 5′ end of SEQ ID NO: 274 or from the 5′ end of SEQ ID NO: 195.

The truncated PGK promoter may, for example, comprise, consist essentially of or consist of the sequence of SEQ ID NO: 196, SEQ ID NO: 197, SEQ ID NO: 198 or SEQ ID NO: 199.

The variant of the truncated PGK promoter may, for example, have at least about 70% sequence identity to a reference truncated PGK promoter. For example, the variant may have at least about 75% or at least about 80% or at least about 85% or at least about 90% or at least about 95% or at least about 96% or at least about 97% or at least about 98% or at least about 99% sequence identity to a reference truncated PGK promoter. The reference truncated PGK promoter may, for example, comprise, consist essentially of or consist of the sequence of SEQ ID NO: 196, SEQ ID NO: 197, SEQ ID NO: 198 or SEQ ID NO: 199.

### CAR

A “chimeric antigen receptor”, “CAR” or “CAR construct” refers to engineered receptors which can confer an antigen specificity onto cells (e.g., immune cells, such as Tregs). In particular, a CAR enables a cell to bind specifically to a particular antigen, e.g., a target molecule such as a target protein, whereupon a signal is generated by the endodomain (comprising an intracellular signalling domain) of the CAR, e.g., a signal resulting in activation of the cell. CARs are also known as artificial T-cell receptors, chimeric T-cell receptors or chimeric immunoreceptors.

The structure of CARs is well-known in the art and several generations of CARs have been produced. For instance, as a minimum a CAR may contain an extracellular antigen-specific targeting region, antigen binding domain, target binding domain or ligand binding domain, which is or forms part of the exodomain (also known as the extracellular domain or ectodomain) of the CAR, a transmembrane domain, and an intracellular signalling domain (which is, or is comprised within, an endodomain). However, the CAR may contain further domains to improve its functionality, e.g., one or more co-stimulatory domains to improve T cell proliferation, cytokine secretion, resistance to apoptosis, and in vivo persistence.

Thus, a chimeric receptor or CAR construct generally comprises a binding domain (which may be viewed as an antigen (i.e., target) or ligand binding domain and the terms binding domain, antigen recognition domain, antigen binding domain and ligand binding domain are used interchangeably herein), optionally a hinge domain, which functions as a spacer to extend the binding domain away from the plasma membrane of the cell (e.g., immune cell) on which it is expressed, a transmembrane domain, an intracellular signalling domain (e.g., the signalling domain from the zeta chain of the CD3 molecule (CD32) of the TCR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g., within two polypeptides which associate with one another.

When the CAR binds its target antigen, this results in the transmission of an activating signal to the cell in which it is expressed. Thus, the CAR directs the specificity of the engineered cells towards the antigen, particularly towards cells expressing the antigen.

The term “directed towards” or “directed against” is synonymous with “specific for” or “anti”. Put another way, the CAR recognises the antigen target molecule. Accordingly, it is meant that the CAR is capable of binding specifically to antigen. In particular, the antigen-binding domain of the CAR is capable of binding specifically to antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing antigen, particularly a target cell expressing antigen on its cell surface. Particularly, “specific” binding means that binding only or mainly occurs to antigen and not to other proteins or polypeptides (i.e. binding to other proteins or polypeptides is insignificant or weaker). Some cross-reaction with other proteins may occur but this level of binding can be considered as background. As mentioned above, the CAR is capable of binding to antigen and of transducing a signal into a cell in which it is expressed. The cell may then be activated and may exert a suppressive effect within the local environment. Activation of a cell expressing a CAR after antigen binding can be determined by an increased level of CD69 as compared to the same cells expressing a CAR in the absence of antigen. For example, an increase of at least 10, 20, 30, 40, 50, 60, 70, 80 or 90% in CD69. Expression levels of CD69 can be determined using standard techniques, for example FACS, using commercially available antibodies (e.g. FITC anti-human CD69 antibody, Biolegend). Thus, CAR function within a cell can be determined by activation status of the cell in which the CAR is expressed, e.g., by determining CD69 expression.

The antigen-binding domain of a CAR may be derived or obtained from any protein or polypeptide which binds (i.e., has affinity for) antigen (e.g. which binds to any region or part of antigen, or alternatively viewed which binds to any epitope within antigen, either in an isolated protein form or when expressed on cells). Particularly, the antigen-binding domain of a CAR may be derived or obtained from any protein or polypeptide which binds (i.e., has affinity for) the extracellular domain of antigen. This may be for example, a ligand of antigen, or a physiological binding protein for antigen, or a part thereof, or a synthetic or derivative protein. The target molecule (antigen) may commonly be expressed on the surface of a cell, for example a target cell, or a cell in the vicinity of a target cell (for a bystander effect), but need not be.

The antigen-binding domain is most commonly derived from antibody variable chains (for example it commonly takes the form of a scFv), but may also be generated from other molecules, such as ligands or other binding molecules.

The CAR is typically expressed as a polypeptide also comprising a signal sequence (also known as a leader sequence), and in particular a signal sequence which targets the CAR to the plasma membrane of the cell. This will generally be positioned next to or close to the antigen binding domain, generally upstream of the antigen binding domain. The extracellular domain, or ectodomain, of the CAR may thus comprise, consist essentially of or consist of a signal sequence and an antigen binding domain.

The antigen binding domain provides the CAR with the ability to bind a predetermined antigen of interest. The antigen binding domain preferably targets an antigen of clinical interest or an antigen at a site of disease.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

Antigens which may be targeted by the CAR include, but are not limited to, antigens expressed on cells associated with transplanted organs, autoimmune diseases, infectious diseases, allergic diseases and inflammatory diseases. It will be understood by a skilled person that where the cell engineered to express the nucleic acid molecule is a Treg cell, or a precursor therefor, due to the bystander effect of Treg cells, the antigen may be simply present and/or expressed at the site of transplantation, inflammation or disease.

Antigens associated with organ transplants and/or cells associated with transplanted organs include, but are not limited to, a HLA antigen present in the transplanted organ but not in the patient, or an antigen whose expression is upregulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

The antigen which may be targeted by the CAR may be HLA-A2. HLA-A2 may particularly be expressed on cells associated with transplanted organs. HLA-A2 may also be referred to herein as HLA-A*02, HLA-A02 and HLA-A*2. HLA-A2 is one particular class I major histocompatibility complex (MHC) allele group at the HLA-A locus. Binders and CARs specific for HLA-A2 are described in WO 2019/056106, WO 2023/019144, WO 2018/001874, WO2020/201230 and WO2020/044055, the contents of which are incorporated by reference. The CAR encoded by the nucleic acid molecule described herein may thus be a CAR or incorporate an antigen binding domain, CDR, VH and/or VL sequence as described in WO 2019/056106, WO 2023/019144, WO 2018/001874 or WO2020/201230 or WO 2020/044055.

Antigens expressed on cells associated with neurodegenerative disease and neuroinflammatory disease include antigens expressed in the central nervous system (CNS), for example those presented on glial cells, motor neurons, oligodendrocytes and Schwann cells. p75NTR (p75 neurotrophin receptor) is an example of an antigen to target CAR-Tregs to the CNS and brain as its expression is normally low in adult brain but is upregulated by injury/trauma/stress and increased p75NTR expression has been detected in a wide variety of neurodegenerative diseases. Binders and CARs specific for p75NTR are described in WO 2023/047098, the contents of which are incorporated herein by reference. The CAR encoded by the nucleic acid molecule described herein may thus be a CAR or incorporate an antigen binding domain, CDR, VH and/or VL sequence as described in WO 2023/047098.

Antigens expressed at the site of an inflammatory lesion, for example present in the extracellular matrix of an inflammatory lesion may be associated with inflammatory and/or autoimmune diseases. For example, the antigen may be a modified protein or proteins present in the extracellular matrix of an inflammatory lesion, for example a citrullinated protein such as citrullinated vimentin, citrullinated filaggrin and citrullinated fibrinogen. For example, the antigen may be citrullinated vimentin, a modified form of vimentin that is specifically found in the joints. This target antigen may be useful for the treatment of rheumatoid arthritis, Alzheimer's disease, sporadic Creutzfeldt-Jakob disease, juvenile idiopathic arthritis, idiopathic pulmonary fibrosis, rheumatoid arthritis associated interstitial lung disease, chronic obstructive pulmonary disease and/or liver fibrosis. For example, citrullinated vimentin may be the target antigen for treatment of rheumatoid arthritis. Binders and CARs specific for citrullinated vimentin are described in WO 2019/157461 and WO 2021/030257, the contents of which are incorporated herein by reference. The CAR encoded by the nucleic acid molecule described herein may thus be a CAR or incorporate an antigen binding domain, CDR, VH and/or VL sequence as described in WO 2019/157461 or WO 2021/030257. Binders and CARs specific for citrullinated vimentin, citrullinated filaggrin and citrullinated fibrinogen are described in WO 2023/010122, the contents of which are incorporated herein by reference. The CAR encoded by the nucleic acid molecule described herein may thus be a CAR or incorporate an antigen binding domain, CDR, VH and/or VL sequence as described in WO 2023/010122.

Antigens associated with type 1 diabetes include antigens present at the site of disease (i.e., the pancreas), for example insulin and antigens expressed on pancreatic beta cells (e.g., ENTPD3, glutamic acid decarboxylase 65 kDA (GAD65) or dipeptidyl-peptidase 6 (DPP6)). Binders and CARs specific for GAD65 are described in WO 2020/097546, WO 2022/240797 and WO 2022/094614, the contents of which are incorporated herein by reference. Binders and CARs specific for DPP6 are disclosed in WO 2022/094614 and WO 2022/187182, the contents of which are incorporated herein by reference. The CAR encoded by the nucleic acid molecule described herein may thus be a CAR or incorporate an antigen binding domain, CDR, VH and/or VL sequence as described in WO 2020/097546, WO 2022/240797, WO 2022/094614, WO 2022/094614 or WO 2022/187182.

The antigen which may be targeted by the CAR may be ENTPD3. ENTPD3 (Ectonucleoside triphosphate diphosphohydrolase 3) is also known as CD39L3, HB6 and NTPDase-3. It is a membrane-bound enzyme that is similar to E-type nucleotidases (NTPases) and is expressed on pancreatic beta cells. The amino acid sequence of human ENTPD3 is shown in SEQ ID NO: 218 and the amino acid sequence of mouse ENTPD3 is shown in SEQ ID NO: 219.

As noted above, the antigen binding domain may be any protein or peptide that possesses the ability to specifically recognize and bind to antigen (e.g. ENTPD3). The antigen binding domain includes any naturally occurring, synthetic, semi-synthetic, or recombinantly produced binding partner for antigen (e.g. ENTPD3). Illustrative antigen-specific targeting domains include antibodies or antibody fragments or derivatives, or ligands for antigen (e.g. ENTPD3).

In an embodiment, the antigen binding domain is, or is derived from, an antibody.

The term “antibody” as used herein refers broadly to any immunological binding agent or molecule that comprises an antigen binding domain, including polyclonal and monoclonal antibodies. Depending on the type of constant domain in the heavy chains, whole antibodies are assigned to one of five major classes: IgA, IgD, IgE, IgG and IgM and the antibodies as described herein may be in any one of these classes. Several of these are further divided into subclasses or isotypes such as IgG1, IgG2, IgG3, IgG4 and the like. Generally, IgG or IgM antibodies are the most common antibodies utilised in physiological settings. As will be understood by those in the art, the term “antibody” extends to all antibodies including whole antibodies, dimeric, trimeric and multimeric antibodies; bispecific antibodies; chimeric antibodies; recombinant and engineered antibodies and fragments thereof.

An antibody-derived binding domain can be a fragment of an antibody or a genetically engineered product of one or more fragments of the antibody, which fragment is involved in binding with the antigen. Examples include a variable region (Fv), a complementarity determining region (CDR), Fab or F(ab′)₂, or the light and heavy chain variable regions can be joined together in a single chain (e.g. as a scFv) and in either orientation (e.g. V_{L}-V_{H} or V_{H}-V_{L}). The V_{L} and/or V_{H} sequences may be modified. In particular, the framework regions may be modified (e.g., substituted, for example to humanise the antigen-binding domain). Other examples include a heavy chain variable region (VH), a light chain variable region (VL), and a single domain antibody (sAb) (which may be referred to as a nanobody). An example of a single domain antibody is a camelid heavy-chain antibody (HCAb) which has an antigen recognition site formed by a single domain, termed VHH.

In an embodiment, the antigen-binding domain is a single chain antibody (scFv). The scFv may be murine, human or humanized scFv.

In an alternative embodiment, the antigen-binding domain is derived from a camelid heavy-chain antibody (HCAb), for example the antigen-binding domain may be a VHH domain of a HCAb. VHHs have a similar structure to that of VH domains from conventional IgGs and include three variable CDRs, although CDRs 1 and 3 often have more amino acids than those of VH domains. In certain embodiments, the VHH domain may comprise one or more CDRs comprising or consisting of the sequence(s) set forth in SEQ ID NOs: 1 to 84 specified herein, or sequence having at least 70%, 75%, 80%, 85%, 90%, 95%, 96%, 97%, 98%, or 99% sequence identity thereto. For example, the VHH domain may comprise one, two or three of the VH CDRs comprising or consisting of the sequence(s) set forth in SEQ ID NOs: 1 to 84 specified herein, or sequences having at least 70%, 75%, 80%, 85%, 90%, 95%, 96%, 97%, 98%, or 99% sequence identity thereto. Where the VHH domain comprises two or three VH CDRs of SEQ ID NOs: 1 to 84, the CDRs may be from the same binder described herein (e.g. all from the A8, B2, B7 binder etc).

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another, in either orientation, directly or via a peptide linker sequence.

Antibodies that specifically bind a predetermined antigen, e.g., ENTPD3, can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule, e.g., to ENTPD3. Phage display libraries of human antibodies are also available. Once identified, the amino acid sequence or polynucleotide sequence coding for the antibody can be isolated and/or determined.

The antigen recognition domain may bind, suitably specifically bind, one or more regions or epitopes within the antigen. An epitope, also known as antigenic determinant, is the part of an antigen that is recognised by an antigen recognition domain (e.g., an antibody). In other words, the epitope is the specific piece of the antigen to which an antibody binds. Suitably, the antigen recognition domain binds, suitably specifically binds, to one region or epitope within the antigen.

The antigen recognition domain may comprise at least one CDR (e.g. CDR3), which can be predicted from an antibody which binds to an antigen, (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13:1882-91).

In this respect, the antigen binding domain may comprise one or more variable heavy chain CDRs, e.g., one, two or three variable heavy chain CDRs. Alternatively, or additionally, the antigen binding domain may comprise one or more variable light chain CDRs, e.g. one, two or three variable light chain CDRs. The antigen binding domain may comprise three heavy chain CDRs and/or three light chain CDRs (and more particularly a heavy chain variable region comprising three CDRs and/or a light chain variable region comprising three CDRs) wherein at least one CDR, optionally all CDRs, may be from an antibody which binds to antigen.

The antigen binding domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two or three variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Optionally, the antigen binding domain comprises three variable heavy chain CDRs (CDR1, CDR2 and CDR3) and/or three variable light chain CDRs (CDR1, CDR2 and CDR3).

The one or more CDRs present within the antigen binding domain may not all be from the same antibody, as long as the domain has the desired binding activity. Thus, one CDR may be predicted from the heavy or light chains of an antibody which binds to antigen (e.g., ENTPD3) whilst another CDR present may be predicted from a different antibody which binds to antigen (e.g., ENTPD3). A combination of CDRs may be used from different antibodies, particularly from antibodies that bind to the same desired region or epitope.

In an embodiment, the antigen binding domain comprises three CDRs predicted from the variable heavy chain sequence of an antibody which binds to a particular antigen and/or three CDRs predicted from the variable light chain sequence of an antibody which binds to a particular antigen (optionally the same antibody).

In an embodiment, the antigen-binding domain comprises VH CDR1, 2 and 3 sequences as set forth in SEQ ID NOs. 1, 2 and 3 respectively and VL CDR1, 2 and 3 sequences as set forth in SEQ ID NOs. 4, 5 and 6 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 85, or a sequence having at least 70% sequence identity thereto, and a VL domain comprising the sequence as set forth in SEQ ID NO: 86, or a sequence having at least 70% sequence identity thereto.

For example, in such an embodiment, the antigen binding domain of the CAR comprises a VH domain comprising the sequence encoded by SEQ ID NO: 167, or a sequence having at least 70% identity to the sequence encoded by SEQ ID NO: 167, and a VL domain comprising the sequence encoded by SEQ ID NO: 168, or a sequence having at least 70% identity to the sequence encoded by SEQ ID NO: 168. For example, the first nucleotide sequence in the nucleic acid molecule described herein may comprise the sequence of SEQ ID NO: 167 and/or the sequence of SEQ ID NO: 168, or a sequence encoding a VH or VL domain having at least 70% identity to the sequence encoded by SEQ ID NO: 167 or SEQ ID NO: 168 respectively.

In an embodiment, the antigen-binding domain comprises VH CDR1, 2 and 3 sequences as set forth in SEQ ID NOs. 7, 8 and 9 respectively and VL CDR1, 2 and 3 sequences as set forth in SEQ ID NOs. 10, 11 and 12 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 87, or a sequence having at least 70% sequence identity thereto, and a VL domain comprising the sequence as set forth in SEQ ID NO: 88, or a sequence having at least 70% sequence identity thereto.

For example, in such an embodiment, the antigen binding domain of the CAR comprises a VH domain comprising the sequence encoded by SEQ ID NO: 169, or a sequence having at least 70% identity to the sequence encoded by SEQ ID NO: 169, and a VL domain comprising the sequence encoded by SEQ ID NO: 170, or a sequence having at least 70% identity to the sequence encoded by SEQ ID NO: 170. For example, the first nucleotide sequence in the nucleic acid molecule described herein may comprise the sequence of SEQ ID NO: 169 and/or the sequence of SEQ ID NO: 170, or a sequence encoding a VH or VL domain having at least 70% identity to the sequence encoded by SEQ ID NO: 169 or SEQ ID NO: 170 respectively.

In an embodiment, the antigen-binding domain comprises VH CDR1, 2 and 3 sequences as set forth in SEQ ID NOs. 13, 14 and 15 respectively and VL CDR1, 2 and 3 sequences as set forth in SEQ ID NOs. 16, 17 and 18 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 89, or a sequence having at least 70% sequence identity thereto, and a VL domain comprising the sequence as set forth in SEQ ID NO: 90, or a sequence having at least 70% sequence identity thereto.

For example, in such an embodiment, the antigen binding domain of the CAR comprises a VH domain comprising the sequence encoded by SEQ ID NO: 171, or a sequence having at least 70% identity to the sequence encoded by SEQ ID NO: 171, and a VL domain comprising the sequence encoded by SEQ ID NO: 172, or a sequence having at least 70% identity to the sequence encoded by SEQ ID NO: 172. For example, the first nucleotide sequence in the nucleic acid molecule described herein may comprise the sequence of SEQ ID NO: 171 and/or the sequence of SEQ ID NO: 172, or a sequence encoding a VH or VL domain having at least 70% identity to the sequence encoded by SEQ ID NO: 171 or SEQ ID NO: 172 respectively.

In an embodiment, the antigen-binding domain comprises VH CDR1, 2 and 3 sequences as set forth in SEQ ID NOs. 19, 20 and 21 respectively and VL CDR1, 2 and 3 sequences as set forth in SEQ ID NOs. 22, 23 and 24 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 91, or a sequence having at least 70% sequence identity thereto, and a VL domain comprising the sequence as set forth in SEQ ID NO: 92, or a sequence having at least 70% sequence identity thereto.

For example, in such an embodiment, the antigen binding domain of the CAR comprises a VH domain comprising the sequence encoded by SEQ ID NO: 173, or a sequence having at least 70% identity to the sequence encoded by SEQ ID NO: 173, and a VL domain comprising the sequence encoded by SEQ ID NO: 174, or a sequence having at least 70% identity to the sequence encoded by SEQ ID NO: 174. For example, the first nucleotide sequence in the nucleic acid molecule described herein may comprise the sequence of SEQ ID NO: 173 and/or the sequence of SEQ ID NO: 174, or a sequence encoding a VH or VL domain having at least 70% identity to the sequence encoded by SEQ ID NO: 173 or SEQ ID NO: 174 respectively.

In an embodiment, the antigen-binding domain comprises VH CDR1, 2 and 3 sequences as set forth in SEQ ID NOs. 25, 26 and 27 respectively and VL CDR1, 2 and 3 sequences as set forth in SEQ ID NOs. 28, 29 and 30 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 93, or a sequence having at least 70% sequence identity thereto, and a VL domain comprising the sequence as set forth in SEQ ID NO: 94, or a sequence having at least 70% sequence identity thereto.

For example, in such an embodiment, the antigen binding domain of the CAR comprises a VH domain comprising the sequence encoded by SEQ ID NO: 175, or a sequence having at least 70% identity to the sequence encoded by SEQ ID NO: 175, and a VL domain comprising the sequence encoded by SEQ ID NO: 176, or a sequence having at least 70% identity to the sequence encoded by SEQ ID NO: 176. For example, the first nucleotide sequence in the nucleic acid molecule described herein may comprise the sequence of SEQ ID NO: 175 and/or the sequence of SEQ ID NO: 176, or a sequence encoding a VH or VL domain having at least 70% identity to the sequence encoded by SEQ ID NO: 175 or SEQ ID NO: 176 respectively.

In an embodiment, the antigen-binding domain comprises VH CDR1, 2 and 3 sequences as set forth in SEQ ID NOs. 31, 32 and 33 respectively and VL CDR1, 2 and 3 sequences as set forth in SEQ ID NOs. 34, 35 and 36 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 95, or a sequence having at least 70% sequence identity thereto, and a VL domain comprising the sequence as set forth in SEQ ID NO: 96, or a sequence having at least 70% sequence identity thereto.

For example, in such an embodiment, the antigen binding domain of the CAR comprises a VH domain comprising the sequence encoded by SEQ ID NO: 177, or a sequence having at least 70% identity to the sequence encoded by SEQ ID NO: 177, and a VL domain comprising the sequence encoded by SEQ ID NO: 178, or a sequence having at least 70% identity to the sequence encoded by SEQ ID NO: 178. For example, the first nucleotide sequence in the nucleic acid molecule described herein may comprise the sequence of SEQ ID NO: 177 and/or the sequence of SEQ ID NO: 178, or a sequence encoding a VH or VL domain having at least 70% identity to the sequence encoded by SEQ ID NO: 177 or SEQ ID NO: 178 respectively.

In an embodiment, the antigen-binding domain comprises VH CDR1, 2 and 3 sequences as set forth in SEQ ID NOs. 37, 38 and 39 respectively and VL CDR1, 2 and 3 sequences as set forth in SEQ ID NOs. 40, 41 and 42 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 97, or a sequence having at least 70% sequence identity thereto, and a VL domain comprising the sequence as set forth in SEQ ID NO: 98, or a sequence having at least 70% sequence identity thereto.

For example, in such an embodiment, the antigen binding domain of the CAR comprises a VH domain comprising the sequence encoded by SEQ ID NO: 179, or a sequence having at least 70% identity to the sequence encoded by SEQ ID NO: 179, and a VL domain comprising the sequence encoded by SEQ ID NO: 180, or a sequence having at least 70% identity to the sequence encoded by SEQ ID NO: 180. For example, the first nucleotide sequence in the nucleic acid molecule described herein may comprise the sequence of SEQ ID NO: 179 and/or the sequence of SEQ ID NO: 180, or a sequence encoding a VH or VL domain having at least 70% identity to the sequence encoded by SEQ ID NO: 179 or SEQ ID NO: 180 respectively.

In an embodiment, the antigen-binding domain comprises VH CDR1, 2 and 3 sequences as set forth in SEQ ID NOs. 43, 44 and 45 respectively and VL CDR1, 2 and 3 sequences as set forth in SEQ ID NOs. 46, 47 and 48 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 99, or a sequence having at least 70% sequence identity thereto, and a VL domain comprising the sequence as set forth in SEQ ID NO: 100, or a sequence having at least 70% sequence identity thereto.

For example, in such an embodiment, the antigen binding domain of the CAR comprises a VH domain comprising the sequence encoded by SEQ ID NO: 181, or a sequence having at least 70% identity to the sequence encoded by SEQ ID NO: 181, and a VL domain comprising the sequence encoded by SEQ ID NO: 182, or a sequence having at least 70% identity to the sequence encoded by SEQ ID NO: 182. For example, the first nucleotide sequence in the nucleic acid molecule described herein may comprise the sequence of SEQ ID NO: 181 and/or the sequence of SEQ ID NO: 182, or a sequence encoding a VH or VL domain having at least 70% identity to the sequence encoded by SEQ ID NO: 181 or SEQ ID NO: 182 respectively.

In an embodiment, the antigen-binding domain comprises VH CDR1, 2 and 3 sequences as set forth in SEQ ID NOs. 49, 50 and 51 respectively and VL CDR1, 2 and 3 sequences as set forth in SEQ ID NOs. 52, 53 and 54 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 101, or a sequence having at least 70% sequence identity thereto, and a VL domain comprising the sequence as set forth in SEQ ID NO: 102, or a sequence having at least 70% sequence identity thereto.

For example, in such an embodiment, the antigen binding domain of the CAR comprises a VH domain comprising the sequence encoded by SEQ ID NO: 183, or a sequence having at least 70% identity to the sequence encoded by SEQ ID NO: 183, and a VL domain comprising the sequence encoded by SEQ ID NO: 184, or a sequence having at least 70% identity to the sequence encoded by SEQ ID NO: 184. For example, the first nucleotide sequence in the nucleic acid molecule described herein may comprise the sequence of SEQ ID NO: 183 and/or the sequence of SEQ ID NO: 184, or a sequence encoding a VH or VL domain having at least 70% identity to the sequence encoded by SEQ ID NO: 183 or SEQ ID NO: 184 respectively.

In an embodiment, the antigen-binding domain comprises VH CDR1, 2 and 3 sequences as set forth in SEQ ID NOs. 55, 56 and 57 respectively and VL CDR1, 2 and 3 sequences as set forth in SEQ ID NOs. 58, 59 and 60 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 103, or a sequence having at least 70% sequence identity thereto, and a VL domain comprising the sequence as set forth in SEQ ID NO: 104, or a sequence having at least 70% sequence identity thereto.

For example, in such an embodiment, the antigen binding domain of the CAR comprises a VH domain comprising the sequence encoded by SEQ ID NO: 185, or a sequence having at least 70% identity to the sequence encoded by SEQ ID NO: 185, and a VL domain comprising the sequence encoded by SEQ ID NO: 186, or a sequence having at least 70% identity to the sequence encoded by SEQ ID NO: 186. For example, the first nucleotide sequence in the nucleic acid molecule described herein may comprise the sequence of SEQ ID NO: 185 and/or the sequence of SEQ ID NO: 186, or a sequence encoding a VH or VL domain having at least 70% identity to the sequence encoded by SEQ ID NO: 185 or SEQ ID NO: 186 respectively.

In an embodiment, the antigen-binding domain comprises VH CDR1, 2 and 3 sequences as set forth in SEQ ID NOs. 61, 62 and 63 respectively and VL CDR1, 2 and 3 sequences as set forth in SEQ ID NOs. 64, 65 and 66 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 105, or a sequence having at least 70% sequence identity thereto, and a VL domain comprising the sequence as set forth in SEQ ID NO: 106, or a sequence having at least 70% sequence identity thereto.

For example, in such an embodiment, the antigen binding domain of the CAR comprises a VH domain comprising the sequence encoded by SEQ ID NO: 187, or a sequence having at least 70% identity to the sequence encoded by SEQ ID NO: 187, and a VL domain comprising the sequence encoded by SEQ ID NO: 188, or a sequence having at least 70% identity to the sequence encoded by SEQ ID NO: 188. For example, the first nucleotide sequence in the nucleic acid molecule described herein may comprise the sequence of SEQ ID NO: 187 and/or the sequence of SEQ ID NO: 188, or a sequence encoding a VH or VL domain having at least 70% identity to the sequence encoded by SEQ ID NO: 187 or SEQ ID NO: 188 respectively.

In an embodiment, the antigen-binding domain comprises VH CDR1, 2 and 3 sequences as set forth in SEQ ID NOs. 67, 68 and 69 respectively and VL CDR1, 2 and 3 sequences as set forth in SEQ ID NOs. 70, 71 and 72 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 107, or a sequence having at least 70% sequence identity thereto, and a VL domain comprising the sequence as set forth in SEQ ID NO: 108, or a sequence having at least 70% sequence identity thereto.

For example, in such an embodiment, the antigen binding domain of the CAR comprises a VH domain comprising the sequence encoded by SEQ ID NO: 189, or a sequence having at least 70% identity to the sequence encoded by SEQ ID NO: 189, and a VL domain comprising the sequence encoded by SEQ ID NO: 190, or a sequence having at least 70% identity to the sequence encoded by SEQ ID NO: 190. For example, the first nucleotide sequence in the nucleic acid molecule described herein may comprise the sequence of SEQ ID NO: 189 and/or the sequence of SEQ ID NO: 190, or a sequence encoding a VH or VL domain having at least 70% identity to the sequence encoded by SEQ ID NO: 189 or SEQ ID NO: 190 respectively.

In an embodiment, the antigen-binding domain comprises VH CDR1, 2 and 3 sequences as set forth in SEQ ID NOs. 73, 74 and 75 respectively and VL CDR1, 2 and 3 sequences as set forth in SEQ ID NOs. 76, 77 and 78 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 109, or a sequence having at least 70% sequence identity thereto, and a VL domain comprising the sequence as set forth in SEQ ID NO: 110, or a sequence having at least 70% sequence identity thereto.

For example, in such an embodiment, the antigen binding domain of the CAR comprises a VH domain comprising the sequence encoded by SEQ ID NO: 191, or a sequence having at least 70% identity to the sequence encoded by SEQ ID NO: 191, and a VL domain comprising the sequence encoded by SEQ ID NO: 192, or a sequence having at least 70% identity to the sequence encoded by SEQ ID NO: 192. For example, the first nucleotide sequence in the nucleic acid molecule described herein may comprise the sequence of SEQ ID NO: 191 and/or the sequence of SEQ ID NO: 192, or a sequence encoding a VH or VL domain having at least 70% identity to the sequence encoded by SEQ ID NO: 191 or SEQ ID NO: 192 respectively.

In an embodiment, the antigen-binding domain comprises VH CDR1, 2 and 3 sequences as set forth in SEQ ID NOs. 79, 80 and 81 respectively and VL CDR1, 2 and 3 sequences as set forth in SEQ ID NOs. 82, 83 and 84 respectively, or the CDRs may contain 1 to 3, or more particularly 1 or 2 amino acid sequence modifications in any of the aforementioned sequences.

More particularly, in such an embodiment the antigen binding domain of the CAR comprises a VH domain comprising the sequence as set forth in SEQ ID NO: 111, or a sequence having at least 70% sequence identity thereto, and a VL domain comprising the sequence as set forth in SEQ ID NO: 112, or a sequence having at least 70% sequence identity thereto.

For example, in such an embodiment, the antigen binding domain of the CAR comprises a VH domain comprising the sequence encoded by SEQ ID NO: 193, or a sequence having at least 70% identity to the sequence encoded by SEQ ID NO: 193, and a VL domain comprising the sequence encoded by SEQ ID NO: 194, or a sequence having at least 70% identity to the sequence encoded by SEQ ID NO: 194. For example, the first nucleotide sequence in the nucleic acid molecule described herein may comprise the sequence of SEQ ID NO: 193 and/or the sequence of SEQ ID NO: 194, or a sequence encoding a VH or VL domain having at least 70% identity to the sequence encoded by SEQ ID NO: 193 or SEQ ID NO: 194 respectively.

Where a CDR does contain an amino acid sequence modification, this may be a deletion, addition, or substitution of an amino acid residue of the CDR sequence as set out in the above-mentioned SEQ ID NOs. More particularly, the modification may be an amino acid substitution, for example a conservative amino acid substitution, e.g., as set out above. A longer CDR may tolerate more amino acid residue modifications. In the case of CDRs which are 5 or more, or 7 or more, amino acid residues long, the modifications may be of 0, 1, 2 or 3 residues, e.g. 2 residues. In general, there may be 0, 1, 2, or 3 modifications to any particular CDR sequence. Further, in an embodiment, CDRs 1 and 2 may be modified, and CDR3 may be unmodified. In another embodiment all 3 CDRs may be modified. In another embodiment, the CDRs are not modified.

The antigen binding domain may be in the form of a scFv comprising the VH and VL domain sequences as set out above, in either order, for example VH-VL. The VH and VL sequences may be linked by a linker sequence.

Suitable linkers can be readily selected and can be of any of a suitable length, such as from 1 amino acid (e.g. Gly) to 30 amino acids, e.g. from any one of 2, 3, 4, 5, 6, 7, 8, 9, or 10 amino acids to any one of 12, 15, 18, 20, 21, 25, 30 amino acids, for example, 5-30, 5-25, 6-25, 10-15, 12-25, 15 to 25 etc.

Exemplary linkers include glycine polymers (G), glycine-serine polymers, glycine-alanine polymers, alanine-serine polymers, and other flexible linkers known in the art, as discussed above. The linker may comprise 1 or more “GS” domains as discussed above.

The linker sequence may be a flexible linker sequence. Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27 (10), 325-330; van Rosmalen et al., 2017, Biochemistry, 56, 6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules.

The most commonly used flexible linkers have sequences primarily composed of Ser and Gly residues (so-called “GS linkers”). However, many other flexible linkers have also been described (see Chen et al, 2013, supra, for example), which may contain additional amino acids such as Thr and/or Ala, and/or Lys and/or Glu which may improve solubility. Any flexible linker known and reported in the art may be used.

Although the length of the linker is not critical, it may in some embodiments be desirable to have a shorter linker sequence. For example, the linker sequence may have a length of no more than 25, optionally no more than 24, 23, 22 or 21 amino acids.

In other embodiments, a longer linker sequence may be desired, for example composed of, or comprising, multiple repeats of a GS domain.

In some embodiments the linker may be from any one of 2, 3, 4, 5 or 6 to any one of 24, 23, 22 or 21 amino acids in length. In other embodiments it may be from any one of 2, 3, 4, 5 or 6 to any one of 21, 20, 19, 18, 17, 16, or 15 amino acids in length. In other embodiments it may be intermediate between these ranges, from example from 6 to 21, 6 to 20, 7 to 20, 8-20, 9-20, 10-20, 8-18, 9-18, 10-18, 9-17, 10-17, 9-16, 10-16 etc. It may accordingly be in a range made up from any of the integers listed above.

The use of GS linkers, or more particularly GS (“Gly-Ser”) domains in linkers, may allow the length of the linker readily to be varied by varying the number of GS domain repeats, and so such linkers represent one class of linkers. However, flexible linkers are not limited to those based on “GS” repeats, and other linkers comprising Ser and Gly residues dispersed throughout the linker sequence have been reported, including in Chen et al., supra.

Accordingly, in one embodiment the linker sequence may comprise at least 40% Gly or Gly and Ser residues.

In another embodiment, the linker sequence may comprise Ser and/or Gly residues, and no more than 15 other amino acid residues, optionally no more than 14, 13, 12, 11, 10, 9, 8, 6, 7, 5, or 4 other amino acid residues. It will be understood than an “other” amino acid residue may be any amino acid which is not Ser or Gly.

Pro residues in linkers tend to confer rigidity and so in one embodiment the linker sequence does not comprise any Pro residues. However, this is not absolute, as depending on the sequence context, a flexible linker sequence may contain one or more Pro residues.

In one embodiment, the linker sequence comprises at least one Gly-Ser domain composed solely of Ser and Gly residues. In such an embodiment, the linker may contain no more than 15 other amino acid residues, optionally no more than 14, 13, 12, 11, 10, 9, 8, 6, 7, 5, or 4 other amino acid residues.

The Gly-Ser domain may have the formula:

(S)q-[(G)m-(S)m]n-(G)p

wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula:

(i)
S-[(G)m-S]n;
(ii)
[(G)m-S]n;
or
(iii)
[(G)m-S]n-(G)p

wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

S-[G-G-G-G-S]n

wherein n is an integer of at least one (optionally 1 to 8, or 1-6, 1-5, 1-4, or 1-3).

A representative sequence GGGS is shown in SEQ ID NO. 220.

A linker sequence may be composed solely of, or may consist of, one or more Gly-Ser domains as described or defined above. However, as noted above, in another embodiment, the linker sequence may comprise one or more Gly-Ser domains, and additional amino acids. The additional amino acids may be at one or both ends of a Gly-Ser domain, or at one or both ends of a stretch of repeating Gly-Ser domains. Thus, the additional amino acid, which may be other amino acids, may lie at one or both ends of the linker sequence, e.g. they may flank the Gly-Ser domain(s). In other embodiments, the additional amino acids may lie between Gly-Ser domains. For example, two Gly-Ser domains may flank a stretch of other amino acids in the linker sequence. Further, as also noted above, in other linkers, GS domains need not be repeated, and G and/or S residues, or a short domain such as GS, may simply be distributed along the length or the sequence.

Representative exemplary linker sequences are listed below:

(SEQ ID NO. 221)
ETSGGGGSRL
(SEQ ID NO. 222)
SGGGGSGGGGSGGGGS
(where GGGGS is SEQ ID NO. 223)
S(GGGGS)₁₋₅
(where GGGGS is SEQ ID NO. 223)
(GGGGS)₁₋₅
(where GGGS is SEQ ID NO. 220)
S(GGGS)₁₋₅
(where GGGS is SEQ ID NO. 220)
(GGGS)₁₋₅
(where GGGGGS is SEQ ID NO. 224)
S(GGGGGS)₁₋₅
(where GGGGGS is SEQ ID NO. 224)
(GGGGGS)₁₋₅
(where GGGGGGS is SEQ ID NO. 225)
S(GGGGGGS)₁₋₅
(where GGGGGGS is SEQ ID NO. 225)
(GGGGGGS)₁₋₅
(SEQ ID NO: 226)
GGGGSGGGGSGGGGS
(SEQ ID NO: 227)
GGGGG
(SEQ ID NO: 228)
GGGGSGGGGS
(SEQ ID NO: 229)
GGGGSGGGGSGGGGSGGGGS
(SEQ ID NO: 230)
GGGGGGG
(SEQ ID NO. 231)
G6
(SEQ ID NO. 232)
G8
(SEQ ID NO. 233)
KESGSVSSEQLAQFRSLD
(SEQ ID NO. 234)
EGKSSGSGSESKST
(SEQ ID NO. 235)
GSAGSAAGSGEF
(SEQ ID NO. 236)
SGGGGSAGSAAGSGEF
(SEQ ID NO. 237)
SGGGLLLLLLLLGGGS
(SEQ ID NO. 238)
SGGGAAAAAAAAGGGS
(SEQ ID NO. 239)
SGGGAAAAAAAAAAAAAAAAGGGS
(SEQ ID NO. 240)
SGALGGLALAGLLLAGLGLGAAGS
(SEQ ID NO. 241)
SLSLSPGGGGGPAR
(SEQ ID NO. 242)
SLSLSPGGGGGPARSLSLSPGGGGG
(SEQ ID NO. 243)
GSSGSS
(SEQ ID NO. 244)
GSSSSSS
(SEQ ID NO. 245)
GGSSSS
(SEQ ID NO. 246)
GSSSSS
(SEQ ID NO. 247)
SGGGGS.

In certain embodiments, the linker has the sequence (GGGGS)₃ (SEQ ID NO: 226).

Although the linker sequences, as defined above, are flexible sequences, the present disclosure includes also other polypeptides, including those which comprise linkers which are not flexible, and/or which do not meet the definitions and requirements set out above.

A further example of a linker that may be used to join the VH and VL domains is KLEEGEFSEARV (SEQ ID NO: 248) or a sequence having at least about 60% identity thereto, for example a sequence having at least about 65% or at least about 70% or at least about 75% or at least about 80% or at least about 85% or at least about 90% or at least about 95% identity thereto. Alternatively, the linker may have the sequence of SEQ ID NO: 248 or a sequence that differs by no more than 6, for example no more than 5 or no more than 4 or no more than 3 or no more than 2 or no more than 1 amino acid. In particular, this linker may be truncated or elongated at the N terminus and/or the C terminus by one or more amino acids (e.g. see Schmiedl A. et al (Protein Eng. 2000 October; 13 (10): 725-34 where this linker is shortened by one amino acid at each terminus compared to SEQ ID NO: 248). In order to improve the stability of this linker and to delete the two putative trypsin cleavage sites (lysine and arginine), the linker may be modified by exchanging lysine (K) against isoleucine (I), arginine (R) against glycine (G) and valine (V) against cysteine (C). The resulting linker has the amino acid sequence ILEEGEFSEAGC (SEQ ID NO: 249). Any peptide linker carrying the consensus amino acid sequence X₁LEEGEFSEAX₂X₃ (SEQ ID NO: 250) wherein X₁ is K or I, X₂ is R or G and X₃ is V or C, may also be used.

Accordingly in one embodiment the antigen binding domain may comprise, or consist of, a VH sequence as set forth in SEQ ID NO. 85 linked via a linker of sequence (X)n, where X is any amino acid and n is an integer of between 15 and 25, to the VL sequence of SEQ ID NO. 86.

Accordingly in one embodiment the antigen binding domain may comprise, or consist of, a VH sequence as set forth in SEQ ID NO. 87 linked via a linker of sequence (X)n, where X is any amino acid and n is an integer of between 15 and 25, to the VL sequence of SEQ ID NO. 88.

Accordingly in one embodiment the antigen binding domain may comprise, or consist of, a VH sequence as set forth in SEQ ID NO. 89 linked via a linker of sequence (X)n, where X is any amino acid and n is an integer of between 15 and 25, to the VL sequence of SEQ ID NO. 90.

Accordingly in one embodiment the antigen binding domain may comprise, or consist of, a VH sequence as set forth in SEQ ID NO. 91 linked via a linker of sequence (X)n, where X is any amino acid and n is an integer of between 15 and 25, to the VL sequence of SEQ ID NO. 92.

Accordingly in one embodiment the antigen binding domain may comprise, or consist of, a VH sequence as set forth in SEQ ID NO. 93 linked via a linker of sequence (X)n, where X is any amino acid and n is an integer of between 15 and 25, to the VL sequence of SEQ ID NO. 94.

Accordingly in one embodiment the antigen binding domain may comprise, or consist of, a VH sequence as set forth in SEQ ID NO. 95 linked via a linker of sequence (X)n, where X is any amino acid and n is an integer of between 15 and 25, to the VL sequence of SEQ ID NO. 96.

Accordingly in one embodiment the antigen binding domain may comprise, or consist of, a VH sequence as set forth in SEQ ID NO. 97 linked via a linker of sequence (X)n, where X is any amino acid and n is an integer of between 15 and 25, to the VL sequence of SEQ ID NO. 98.

Accordingly in one embodiment the antigen binding domain may comprise, or consist of, a VH sequence as set forth in SEQ ID NO. 99 linked via a linker of sequence (X)n, where X is any amino acid and n is an integer of between 15 and 25, to the VL sequence of SEQ ID NO. 100.

Accordingly in one embodiment the antigen binding domain may comprise, or consist of, a VH sequence as set forth in SEQ ID NO. 101 linked via a linker of sequence (X)n, where X is any amino acid and n is an integer of between 15 and 25, to the VL sequence of SEQ ID NO. 102.

Accordingly in one embodiment the antigen binding domain may comprise, or consist of, a VH sequence as set forth in SEQ ID NO. 103 linked via a linker of sequence (X)n, where X is any amino acid and n is an integer of between 15 and 25, to the VL sequence of SEQ ID NO. 104.

Accordingly in one embodiment the antigen binding domain may comprise, or consist of, a VH sequence as set forth in SEQ ID NO. 105 linked via a linker of sequence (X)n, where X is any amino acid and n is an integer of between 15 and 25, to the VL sequence of SEQ ID NO. 106.

Accordingly in one embodiment the antigen binding domain may comprise, or consist of, a VH sequence as set forth in SEQ ID NO. 107 linked via a linker of sequence (X)n, where X is any amino acid and n is an integer of between 15 and 25, to the VL sequence of SEQ ID NO. 108.

Accordingly in one embodiment the antigen binding domain may comprise, or consist of, a VH sequence as set forth in SEQ ID NO. 109 linked via a linker of sequence (X)n, where X is any amino acid and n is an integer of between 15 and 25, to the VL sequence of SEQ ID NO. 110.

Accordingly in one embodiment the antigen binding domain may comprise, or consist of, a VH sequence as set forth in SEQ ID NO. 111 linked via a linker of sequence (X)n, where X is any amino acid and n is an integer of between 15 and 25, to the VL sequence of SEQ ID NO. 112.

In this respect, in one embodiment, the antigen binding domain of the CAR comprises or consists of the sequence as set forth in SEQ ID NO: 113 or a sequence having at least 70% or 80% identity thereto.

In another embodiment, in one embodiment, the antigen binding domain of the CAR comprises or consists of the sequence as set forth in SEQ ID NO: 114 or a sequence having at least 70% or 80% identity thereto.

In a further embodiment, in one embodiment, the antigen binding domain of the CAR comprises or consists of the sequence as set forth in SEQ ID NO: 115 or a sequence having at least 70% or 80% identity thereto.

In a further embodiment, in one embodiment, the antigen binding domain of the CAR comprises or consists of the sequence as set forth in SEQ ID NO: 116 or a sequence having at least 70% or 80% identity thereto.

In a further embodiment, in one embodiment, the antigen binding domain of the CAR comprises or consists of the sequence as set forth in SEQ ID NO: 117 or a sequence having at least 70% or 80% identity thereto.

In a further embodiment, in one embodiment, the antigen binding domain of the CAR comprises or consists of the sequence as set forth in SEQ ID NO: 118 or a sequence having at least 70% or 80% identity thereto.

In a further embodiment, in one embodiment, the antigen binding domain of the CAR comprises or consists of the sequence as set forth in SEQ ID NO: 119 or a sequence having at least 70% or 80% identity thereto.

In a further embodiment, in one embodiment, the antigen binding domain of the CAR comprises or consists of the sequence as set forth in SEQ ID NO: 120 or a sequence having at least 70% or 80% identity thereto.

In a further embodiment, in one embodiment, the antigen binding domain of the CAR comprises or consists of the sequence as set forth in SEQ ID NO: 121 or a sequence having at least 70% or 80% identity thereto.

In a further embodiment, in one embodiment, the antigen binding domain of the CAR comprises or consists of the sequence as set forth in SEQ ID NO: 122 or a sequence having at least 70% or 80% identity thereto.

In a further embodiment, in one embodiment, the antigen binding domain of the CAR comprises or consists of the sequence as set forth in SEQ ID NO: 123 or a sequence having at least 70% or 80% identity thereto.

In a further embodiment, in one embodiment, the antigen binding domain of the CAR comprises or consists of the sequence as set forth in SEQ ID NO: 124 or a sequence having at least 70% or 80% identity thereto.

In a further embodiment, in one embodiment, the antigen binding domain of the CAR comprises or consists of the sequence as set forth in SEQ ID NO: 125 or a sequence having at least 70% or 80% identity thereto.

In a further embodiment, in one embodiment, the antigen binding domain of the CAR comprises or consists of the sequence as set forth in SEQ ID NO: 126 or a sequence having at least 70% or 80% identity thereto.

In one embodiment, the antigen binding domain of the CAR comprises or consists of the amino acid sequence encoded by a sequence as set forth in SEQ ID NO: 153 or an amino acid sequence having at least 80% identity to the amino acid sequence encoded by the sequence set forth in SEQ ID NO: 153. For example, the antigen binding domain of the CAR may comprise of consist of the amino acid sequence encoded by the sequence remaining after the sequence encoding the signal sequence and/or any tag sequence (e.g myc-tag and/or his-tag) is removed from SEQ ID NO: 153 or variant thereof. For example, the first nucleotide sequence encoding the CAR may comprise the sequence set forth in SEQ ID NO: 153 or a sequence encoding an amino acid sequence having at least 80% identity to the amino acid sequence encoded by the sequence set forth in SEQ ID NO: 153. For example, the first nucleotide sequence encoding the CAR may comprise the sequence remaining after the sequence encoding the signal sequence any/or any tag sequence is removed from SEQ ID NO: 153.

In one embodiment, the antigen binding domain of the CAR comprises or consists of the amino acid sequence encoded by a sequence as set forth in SEQ ID NO: 154 or an amino acid sequence having at least 80% identity to the amino acid sequence encoded by the sequence set forth in SEQ ID NO: 154. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 154 or variant thereof. For example, the first nucleotide sequence encoding the CAR may comprise the sequence set forth in SEQ ID NO: 154 or a sequence encoding an amino acid sequence having at least 80% identity to the amino acid sequence encoded by the sequence set forth in SEQ ID NO: 154. For example, the first nucleotide sequence encoding the CAR may comprise the sequence remaining after the sequence encoding the signal sequence any/or any tag sequence is removed from SEQ ID NO: 154.

In one embodiment, the antigen binding domain of the CAR comprises or consists of the amino acid sequence encoded by a sequence as set forth in SEQ ID NO: 155 or an amino acid sequence having at least 80% identity to the amino acid sequence encoded by the sequence set forth in SEQ ID NO: 155. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 155 or variant thereof. For example, the first nucleotide sequence encoding the CAR may comprise the sequence set forth in SEQ ID NO: 155 or a sequence encoding an amino acid sequence having at least 80% identity to the amino acid sequence encoded by the sequence set forth in SEQ ID NO: 155. For example, the first nucleotide sequence encoding the CAR may comprise the sequence remaining after the sequence encoding the signal sequence any/or any tag sequence is removed from SEQ ID NO: 155.

In one embodiment, the antigen binding domain of the CAR comprises or consists of the amino acid sequence encoded by a sequence as set forth in SEQ ID NO: 156 or an amino acid sequence having at least 80% identity to the amino acid sequence encoded by the sequence set forth in SEQ ID NO: 156. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 156. For example, the first nucleotide sequence encoding the CAR may comprise the sequence set forth in SEQ ID NO: 156 or a sequence encoding an amino acid sequence having at least 80% identity to the amino acid sequence encoded by the sequence set forth in SEQ ID NO: 156. For example, the first nucleotide sequence encoding the CAR may comprise the sequence remaining after the sequence encoding the signal sequence any/or any tag sequence is removed from SEQ ID NO: 156.

In one embodiment, the antigen binding domain comprises or consists of the CAR is the amino acid sequence encoded by a sequence as set forth in SEQ ID NO: 157 or an amino acid sequence having at least 80% identity to the amino acid sequence encoded by the sequence set forth in SEQ ID NO: 157. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 157 or variant thereof. For example, the first nucleotide sequence encoding the CAR may comprise the sequence set forth in SEQ ID NO: 157 or a sequence encoding an amino acid sequence having at least 80% identity to the amino acid sequence encoded by the sequence set forth in SEQ ID NO: 157. For example, the first nucleotide sequence encoding the CAR may comprise the sequence remaining after the sequence encoding the signal sequence any/or any tag sequence is removed from SEQ ID NO: 157.

In one embodiment, the antigen binding domain of the CAR comprises or consists of the amino acid sequence encoded by a sequence as set forth in SEQ ID NO: 158 or an amino acid sequence having at least 80% identity to the amino acid sequence encoded by the sequence set forth in SEQ ID NO: 158. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 158 or variant thereof. For example, the first nucleotide sequence encoding the CAR may comprise the sequence set forth in SEQ ID NO: 158 or a sequence encoding an amino acid sequence having at least 80% identity to the amino acid sequence encoded by the sequence set forth in SEQ ID NO: 158. For example, the first nucleotide sequence encoding the CAR may comprise the sequence remaining after the sequence encoding the signal sequence any/or any tag sequence is removed from SEQ ID NO: 158.

In one embodiment, the antigen binding domain of the CAR comprises or consists of the amino acid sequence encoded by a sequence as set forth in SEQ ID NO: 159 or an amino acid sequence having at least 80% identity to the amino acid sequence encoded by the sequence set forth in SEQ ID NO: 159. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 159 or variant thereof. For example, the first nucleotide sequence encoding the CAR may comprise the sequence set forth in SEQ ID NO: 159 or a sequence encoding an amino acid sequence having at least 80% identity to the amino acid sequence encoded by the sequence set forth in SEQ ID NO: 159. For example, the first nucleotide sequence encoding the CAR may comprise the sequence remaining after the sequence encoding the signal sequence any/or any tag sequence is removed from SEQ ID NO: 159.

In one embodiment, the antigen binding domain of the CAR comprises or consists of the amino acid sequence encoded by a sequence as set forth in SEQ ID NO: 160 or an amino acid sequence having at least 80% identity to the amino acid sequence encoded by the sequence set forth in SEQ ID NO: 160. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 160 or variant thereof. For example, the first nucleotide sequence encoding the CAR may comprise the sequence set forth in SEQ ID NO: 160 or a sequence encoding an amino acid sequence having at least 80% identity to the amino acid sequence encoded by the sequence set forth in SEQ ID NO: 160. For example, the first nucleotide sequence encoding the CAR may comprise the sequence remaining after the sequence encoding the signal sequence any/or any tag sequence is removed from SEQ ID NO: 160.

In one embodiment, the antigen binding domain of the CAR comprises or consists of the amino acid sequence encoded by a sequence as set forth in SEQ ID NO: 161 or an amino acid sequence having at least 80% identity to the amino acid sequence encoded by the sequence set forth in SEQ ID NO: 161. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 161 or variant thereof. For example, the first nucleotide sequence encoding the CAR may comprise the sequence set forth in SEQ ID NO: 161 or a sequence encoding an amino acid sequence having at least 80% identity to the amino acid sequence encoded by the sequence set forth in SEQ ID NO: 161. For example, the first nucleotide sequence encoding the CAR may comprise the sequence remaining after the sequence encoding the signal sequence any/or any tag sequence is removed from SEQ ID NO: 161.

In one embodiment, the antigen binding domain of the CAR comprises or consists of the amino acid sequence encoded by a sequence as set forth in SEQ ID NO: 162 or an amino acid sequence having at least 80% identity to the amino acid sequence encoded by the sequence set forth in SEQ ID NO: 162. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 162 or variant thereof. For example, the first nucleotide sequence encoding the CAR may comprise the sequence set forth in SEQ ID NO: 162 or a sequence encoding an amino acid sequence having at least 80% identity to the amino acid sequence encoded by the sequence set forth in SEQ ID NO: 162. For example, the first nucleotide sequence encoding the CAR may comprise the sequence remaining after the sequence encoding the signal sequence any/or any tag sequence is removed from SEQ ID NO: 162.

In one embodiment, the antigen binding domain of the CAR comprises or consists of the amino acid sequence encoded by a sequence as set forth in SEQ ID NO: 163 or an amino acid sequence having at least 80% identity to the amino acid sequence encoded by the sequence set forth in SEQ ID NO: 163. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 163 or variant thereof. For example, the first nucleotide sequence encoding the CAR may comprise the sequence set forth in SEQ ID NO: 163 or a sequence encoding an amino acid sequence having at least 80% identity to the amino acid sequence encoded by the sequence set forth in SEQ ID NO: 163. For example, the first nucleotide sequence encoding the CAR may comprise the sequence remaining after the sequence encoding the signal sequence any/or any tag sequence is removed from SEQ ID NO: 163.

In one embodiment, the antigen binding domain of the CAR comprises or consists of the amino acid sequence encoded by a sequence as set forth in SEQ ID NO: 164 or an amino acid sequence having at least 80% identity to the amino acid sequence encoded by the sequence set forth in SEQ ID NO: 164. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 164 or variant thereof. For example, the first nucleotide sequence encoding the CAR may comprise the sequence set forth in SEQ ID NO: 164 or a sequence encoding an amino acid sequence having at least 80% identity to the amino acid sequence encoded by the sequence set forth in SEQ ID NO: 164. For example, the first nucleotide sequence encoding the CAR may comprise the sequence remaining after the sequence encoding the signal sequence any/or any tag sequence is removed from SEQ ID NO: 164.

In one embodiment, the antigen binding domain of the CAR comprises or consists of the amino acid sequence encoded by a sequence as set forth in SEQ ID NO: 165 or an amino acid sequence having at least 80% identity to the amino acid sequence encoded by the sequence set forth in SEQ ID NO: 165. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 165 or variant thereof. For example, the first nucleotide sequence encoding the CAR may comprise the sequence set forth in SEQ ID NO: 165 or a sequence encoding an amino acid sequence having at least 80% identity to the amino acid sequence encoded by the sequence set forth in SEQ ID NO: 165. For example, the first nucleotide sequence encoding the CAR may comprise the sequence remaining after the sequence encoding the signal sequence any/or any tag sequence is removed from SEQ ID NO: 165.

In one embodiment, the antigen binding domain of the CAR comprises or consists of the amino acid sequence encoded by a sequence as set forth in SEQ ID NO: 166 or an amino acid sequence having at least 80% identity to the amino acid sequence encoded by the sequence set forth in SEQ ID NO: 166. For example, the antigen binding domain of the CAR may comprise or consist of the amino acid sequence encoded by the sequence remaining after the sequence encoding the signal sequence and/or any tag sequence (e.g. myc-tag and/or his-tag) is removed from SEQ ID NO: 166 or variant thereof. For example, the first nucleotide sequence encoding the CAR may comprise the sequence set forth in SEQ ID NO: 166 or a sequence encoding an amino acid sequence having at least 80% identity to the amino acid sequence encoded by the sequence set forth in SEQ ID NO: 166. For example, the first nucleotide sequence encoding the CAR may comprise the sequence remaining after the sequence encoding the signal sequence any/or any tag sequence is removed from SEQ ID NO: 166.

In a further embodiment, the CAR construct may comprise, or consist of, a sequence as set forth in any one of SEQ ID NOs: 127 to 152, or a variant thereof, e.g. a sequence having at least 70% or 80% identity thereto. In certain embodiments, the CAR construct may comprise, or consist of, a sequence as set forth in SEQ ID NO: 128, or a variant thereof, e.g. a sequence having at least 70% or 80% identity thereto. In certain embodiments, the CAR construct may comprise, or consist of, a sequence as set forth in SEQ ID NO: 130, or a variant thereof, e.g. a sequence having at least 70% or 80% identity thereto. In certain embodiments, the CAR construct may comprise, or consist of, a sequence as set forth in SEQ ID NO: 131, or a variant thereof, e.g. a sequence having at least 70% or 80% identity thereto. In certain embodiments, the CAR construct may comprise, or consist of, a sequence as set forth in SEQ ID NO: 133, or a variant thereof, e.g. a sequence having at least 70% or 80% identity thereto. In certain embodiments, the CAR construct may comprise, or consist of, a sequence as set forth in SEQ ID NO: 139, or a variant thereof, e.g. a sequence having at least 70% or 80% identity thereto. In certain embodiments, the CAR construct may comprise, or consist of, a sequence as set forth in SEQ ID NO: 143, or a variant thereof, e.g. a sequence having at least 70% or 80% identity thereto.

The variant sequences disclosed and described herein, including the variant CAR, VH, VL, antigen binding domain sequences, may have at least 75, 80, 85, 90, 92, 95, 96, 97, 98, or 99% sequence identity to the specified SEQ ID NOs. The variant sequences disclosed and described herein may be functional variants or functional fragments of the reference CAR, VH, VL, antigen binding domain sequences described herein.

The CAR also optionally comprises a hinge domain to hold the extracellular domain, particularly the antigen binding domain, away from the cell surface, and further comprises a transmembrane domain. The hinge and transmembrane domains may comprise the hinge and transmembrane sequences from any protein which has a hinge domain and/or a transmembrane domain, including any of the type I, type II or type Ill transmembrane proteins. The hinge domain may be selected from the hinge regions of CD28, CD8alpha, CD4, CD7, CH2CH3, an immunoglobulin, or a part or variant thereof. Typically, the hinge may be derived from CD8, particularly, CD8alpha, or from CH2CH3. In one embodiment, the hinge may comprise one or more cysteine residues e.g., to allow disulphide bonding. For example, the CD8 hinge may comprise one or more cysteine residues e.g., one cysteine residue, two cysteine residues or three cysteine residues.

The transmembrane domain of the CAR may also comprise an artificial hydrophobic sequence. The transmembrane domains of the CAR may be selected so as not to dimerize. Additional transmembrane domains will be apparent to those of skill in the art. Examples of transmembrane (TM) regions used in CAR constructs are: 1) The CD28 TM region (Pule et al, Mol Ther, 2005 November; 12(5):933-41; Brentjens et al, CCR, 2007 Sep. 15; 13(18 Pt 1):5426-35; Casucci et al, Blood, 2013 Nov. 14; 122(20):3461-72); 2) The OX40 TM region (Pule et al, Mol Ther, 2005 November; 12(5):933-41); 3) The 4-1BB TM region (Brentjens et al, CCR, 2007 Sep. 15; 13(18 Pt 1):5426-35); 4). The CD3 zeta TM region (Pule et al, Mol Ther, 2005 November; 12(5):933-41; Savoldo B, Blood, 2009 Jun. 18; 113(25):6392-402); 5) The CD8α TM region (Maher et al, Nat Biotechnol, 2002 January; 20 (1): 70-5; Imai C, Leukemia, 2004 April; 18(4):676-84; Brentjens et al, CCR, 2007 Sep. 15; 13(18 Pt 1):5426-35; Milone et al, Mol Ther, 2009 August; 17(8):1453-64). Other transmembrane domains which may be used include those from ICOS, CD4, CD45, CD9, CD16, CD22, CD33, CD64, CD80, CD86, CD154 or CH2CH3. Optionally, the transmembrane domain may be derived from CD4, CD28, CD8α or CH2CH3.

In one embodiment, the CAR may not comprise a dimerisation domain which binds to a regulating molecule. A regulating molecule is any molecule that can bind to at least one dimerisation domain in a CAR and can prevent the interaction, or cause the interaction, of a pair of dimerisation domains. Examples of regulating molecules include soluble proteins (e.g., cytokines, TGF-beta, VEGF) or small molecules. In a further embodiment, when dimerisation with other CAR molecules occurs, it may not be controlled. In another embodiment, monovalent binding of the CAR to an antigen may allow activation of the cell in which the CAR is expressed.

A hinge domain may conveniently be obtained from the same protein as the transmembrane domain. In one embodiment, where the transmembrane domain is derived from the CD8α transmembrane domain, the hinge domain is derived from the CD8α hinge domain. In an alternative embodiment, where the transmembrane domain is derived from the CH2CH3 transmembrane domain, the hinge domain is derived from the CH2CH3 hinge domain.

Alternatively, the hinge domain may be obtained from a different protein to the transmembrane domain. For example, the hinge domain may be derived from the CH2CH3 hinge domain and the transmembrane domain may be derived from the CD28 transmembrane domain.

For example, the hinge domain may be derived from the CD8α hinge domain and may comprise the amino acid sequence shown in SEQ ID NO: 251, or a variant thereof which is at least 80% identical to SEQ ID NO; 251. Suitably, the variant may be at least 85, 90, 95, 97, 98 or 99% identical to SEQ ID NO: 251. An example of a modified CD8α hinge domain is shown in SEQ ID NO: 252.

For example, the hinge domain may comprise the amino acid sequence shown in SEQ ID NO: 253 or encoded by the nucleotide sequence shown in SEQ ID NO: 254, or a variant thereof which is at least 80% identical to SEQ ID NO: 253 or SEQ ID NO: 253. Suitably, the variant may be at least 85, 90, 95, 97, 98 or 99% identical to SEQ ID NO: 253 or SEQ ID NO: 254.

For example, the transmembrane domain may be derived from the CD8α transmembrane domain and may comprise the amino acid sequence shown as SEQ ID NO: 255 which represents amino acids 183 to 203 of human CD8α, or a variant which is at least 80% identical to SEQ ID NO: 255. Suitably, the variant may be at least 85, 90, 95, 97, 98 or 99% identical to SEQ ID NO: 255.

The CD8α transmembrane domain may be combined with a CD8α hinge domain. In an embodiment, the CAR comprises a combined CD8α hinge and transmembrane domain sequence as shown in SEQ ID NO. 256 or SEQ ID NO. 257, or a variant thereof which has at least 80% sequence identity thereto. The variant may be at least 85, 90, 95, 97, 98 or 99% identical to SEQ ID NO: 256 or SEQ ID NO. 257 respectively. SEQ ID NO. 256 comprises a modified hinge domain comprising 2 amino acid modifications of cysteine residues relative to the wild-type CD8α hinge sequence. The modified CD8α hinge domain sequence is shown in SEQ ID NO. 256. The wildtype CD8α hinge and transmembrane domain sequence is shown in SEQ ID NO. 257. The 6 amino acids at the end of SEQ ID NO. 256 and 257, when present, are not located in the membrane and form part of the endodomain of the CAR. A variant of such hinge sequences having at least 80% sequence identity to SEQ ID NO. 256 or 257 may be used.

For example, the hinge domain may be derived from the CH2CH3 hinge domain and may comprise the sequence shown in SEQ ID NO. 258 or 259 or a variant thereof which is at least 80% identical to SEQ ID NO. 258 or 259 respectively. The variant may be at least 85, 90, 95, 97, 98 or 99% identical to SEQ ID NO. 258 or 259 respectively.

Alternatively, an example of a CD28 hinge and transmembrane sequence which may be used is SEQ ID NO: 260 or a variant thereof which is at least 80% identical to SEQ ID NO: 260. The variant may be at least 85, 90, 95, 97, 98 or 99% identical to SEQ ID NO: 260.

By way of further example, the CAR may comprise a native or modified CD8α hinge domain and a CD28 transmembrane domain, or a CD28 hinge domain and CD8α transmembrane domain, for example based on the sequences given above.

In one embodiment, a CH2CH3 hinge sequence comprising one or more cysteine residues may be used. For example, a CH2CH3 hinge sequence comprising one, two, three, four or more cysteine residues. Other hinge domains which may be used include those from CD4, CD7, or an immunoglobulin, or a part or variant thereof. These hinge domains may comprise one or more cysteine residues, for example one, two, three, four or more cysteine residues.

In one embodiment, the transmembrane domain may be derived from the CD4 transmembrane domain and may comprise the sequence shown in SEQ ID NO: 261 or a variant thereof which is at least 80% identical to SEQ ID NO: 261. The variant may be at least 85, 90, 95, 97, 98 or 99% identical to SEQ ID NO: 261.

The CAR may further comprise a signal (or alternatively termed, leader) sequence which targets it to the endoplasmic reticulum pathway for expression on the cell surface. An illustrative signal/leader sequence is MALPVTALLLPLALLLHAAAP as shown in SEQ ID NO. 262. This comprises a single amino acid substitution compared to the wild type CD8α sequence MALPVTALLLPLALLLHAARP as shown in SEQ ID NO. 263. Either sequence, or a variant sequence having at least 70% sequence identity thereto may be used. For example, a variant sequence may have at least 75, 80, 85, 90, 95, 97, 98 or 99% sequence identity thereto.

The endodomain of a CAR as described herein comprises motifs necessary to transduce the effector function signal and direct a cell expressing the CAR to perform its specialized function upon antigen binding. Particularly, the endodomain may comprise one or more (e.g. two or three) Immunoreceptor tyrosine-based activation motifs (ITAMs), typically comprising the amino acid sequence of YXXL/I, where X can be any amino acid. Examples of intracellular signaling domains include, but are not limited to, ζ chain endodomain of the T-cell receptor or any of its homologs (e.g., n chain, FcεR1γ and β chains, MB1 (Igα) chain, B29 (Igβ) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcyRIII, FcsRI, cytoplasmic tails of Fc receptors, immunoreceptor tyrosine-based activation motif (ITAM) bearing cytoplasmic receptors or combinations thereof.

Commonly, the intracellular signaling domain comprises the intracellular signaling domain of a human CD3 zeta chain. The sequence of the intracellular signaling domain of human CD3 zeta chain is set out in SEQ ID NO. 264. The CAR may comprise a CD3ζ signalling domain comprising or consisting of a sequence as set out in SEQ ID NO. 264 or a sequence having at least 80, 85, 90, 95, 97, 98 or 99% identity to SEQ ID NO: 264. In an embodiment the signaling domain comprises or consists of SEQ ID NO. 264.

Other signaling domains which may be used include the signaling domains of CD28 or CD27 or variants thereof. Additional intracellular signaling domains will be apparent to those of skill in the art and may be used in connection with alternate embodiments of the invention. In one embodiment, the present CAR may not comprise a costimulatory domain derived from 4-1BB within the endodomain.

The present CAR may comprise a compound endodomain comprising a fusion of the intracellular part of a T-cell co-stimulatory molecule to that of e.g. CD3ζ. Such a compound endodomain may be referred to as a second-generation CAR which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. The CAR endodomain may also comprise one or more TNF receptor family signalling domain, such as the signalling domain of ICOS, (CD134) OX40, 4-1BB, CD27 or TNFRSF25, or a part or variant thereof, although optionally the CAR may not comprise an endodomain comprising the signalling domains of both CD28 and 4-1BB.

An intracellular signaling domain of CD28 which may be used as a co-stimulatory domain is shown in SEQ ID NO. 265. Illustrative sequences for OX40, 4-1BB, ICOS and TNFRSF25 signalling domains are shown in SEQ ID NO: 266 to 269. The CAR may comprise one or more co-stimulatory domains comprising or consisting of the sequence of any one of SEQ ID NO: 265, 266, 267, 268, 269 or a variant thereof having at least 80, 85, 90, 95, 97, 98 or 99% sequence identity thereto.

In certain embodiments, the transmembrane domain and the intracellular signalling domain derived from a T-cell co-stimulatory molecule may be derived from the same protein. For example, in certain embodiments, the transmembrane domain and intracellular signalling domain derived from a T-cell co-stimulatory molecule may be derived from CD28. For example, the CAR may comprise a combined CD28 transmembrane and CD28 intracellular signalling domain as shown in SEQ ID NO: 270, or a variant thereof having at least 80% sequence identity thereto. The variant may be at least 85, 90, 95, 97, 98 or 99% identical to SEQ ID NO: 270.

In one embodiment, the CAR comprises a human CD8 hinge domain or a variant thereof and a human CD8 transmembrane domain. Alternatively, or additionally, the CAR comprises an endodomain comprising, consisting essentially of or consisting of a human CD28 co-stimulatory domain and a human CD3 zeta signalling domain.

In one embodiment the CAR comprises a hinge, transmembrane, and intracellular (or endo) domains as follows:
(i) a CH2CH3 hinge sequence comprising or consisting of the sequence as set forth in SEQ ID NO. 258 or SEQ ID NO. 259, or a sequence having at least 80% sequence identity thereto;
(ii) a CD28 transmembrane and co-stimulatory domain comprising or consisting of the sequence as set forth in SEQ ID NO. 270, or a sequence having at least 80% sequence identity thereto;
(iii) a CD3ζ signalling domain comprising or consisting of the sequence as set forth in SEQ ID NO. 264, or a sequence having at least 80% sequence identity thereto.

In an alternative embodiment the CAR comprises a hinge, transmembrane, and intracellular (or endo) domains as follows:
(i) a CD8α hinge and transmembrane domain sequence comprising or consisting of the sequence as set forth in SEQ ID NO. 256 or SEQ ID NO: 257, or a sequence having at least 80% sequence identity thereto;
(ii) a CD28 co-stimulatory domain comprising or consisting of the sequence as set forth in SEQ ID NO. 265, or a sequence having at least 80% sequence identity thereto;
(iii) a CD34 signalling domain comprising or consisting of the sequence as set forth in SEQ ID NO. 270, or a sequence having at least 80% sequence identity thereto.

The CAR, as encoded and expressed, may further comprise a leader sequence comprising or consisting of a sequence as set out in SEQ ID NO. 263, or a sequence having at least 80% sequence identity thereto.

The antigen binding domain of the CAR may comprise or consist of a sequence as set out in SEQ ID NO. 113, 114, 115, 116, 117, 118, 119, 120, 121, 122, 123, 124, 125, 126 or a sequence having at least 80% sequence identity thereto which is capable of binding to ENTPD3.

Thus, in its entirety one representative CAR may comprise:
(i) a leader sequence comprising or consisting of a sequence as set out in SEQ ID NO. 263, or a sequence having at least 80% sequence identity thereto;
(ii) an antigen binding domain comprising or consisting of a sequence as set out in SEQ ID NO. 113, 114, 115, 116, 117, 118, 119, 120, 121, 122, 123, 124, 125, 126 or a sequence having at least 80% sequence identity thereto;
(iii) a CH2CH3 hinge domain sequence comprising or consisting of the sequence as set forth in SEQ ID NO. 258 or 259, or a sequence having at least 80% sequence identity thereto;
(iv) a CD28 transmembrane and co-stimulatory domain comprising or consisting of the sequence as set forth in SEQ ID NO. 270, or a sequence having at least 80% sequence identity thereto;
(v) a CD33 signalling domain comprising or consisting of the sequence as set forth in SEQ ID NO. 264, or a sequence having at least 80% sequence identity thereto.

An alternative representative CAR may comprise:
(i) a leader sequence comprising or consisting of a sequence as set out in SEQ ID NO. 263, or a sequence having at least 80% sequence identity thereto;
(ii) an antigen binding domain comprising or consisting of a sequence as set out in SEQ ID NO. 113, 114, 115, 116, 117, 118, 119, 120, 121, 122, 123, 124, 125, 126, or a sequence having at least 80% sequence identity thereto;
(iii) a CD8 hinge and transmembrane domain sequence comprising or consisting of the sequence as set forth in SEQ ID NO. 256 or 257, or a sequence having at least 80% sequence identity thereto;
(iv) a CD28 co-stimulatory domain comprising or consisting of the sequence as set forth in SEQ ID NO. 265, or a sequence having at least 80% sequence identity thereto;
(v) a CD3ζ signalling domain comprising or consisting of the sequence as set forth in SEQ ID NO. 264, or a sequence having at least 80% sequence identity thereto.

The CAR of the present invention may comprise, or consist of, any one or more of the sequences as set out in SEQ ID NOs. 127 to 152, or a variant thereof having at least about 70% or 80% identity thereto. For example, any variant may have at least about 85, 90, 95, 97, 98 or 99% identity to SEQ ID NOs 127 to 152. The CARs of SEQ ID NOs 127 to 152, or variants thereof, may further comprise a signal sequence, for example a signal sequence having the sequence set out in SEQ ID NO: 262 or 263.

The variant sequences disclosed and described herein, including the variant CAR domain sequences (e.g. leader sequence, antigen binding domain sequence, hinge domain sequence, transmembrane domain sequence, co-stimulatory domain sequence and/or intracellular signalling sequence), may have at least 75, 80, 85, 90, 92, 95, 96, 97, 98, or 99% sequence identity to the specified SEQ ID NOs. The variant sequences disclosed and described herein may be functional variants or functional fragments of the reference domain sequences described herein.

The CAR may be capable of binding to ENTPD3 and of transducing a signal into a cell in which it is expressed.

The CAR may be a human CAR. By “human CAR” it is meant that all domains of the CAR (e.g. hinge, transmembrane, co-stimulatory and/or signalling domains) are all derived from human proteins.

The cell may express only one type of CAR, i.e., where the cell expresses more than one CAR molecule, the amino acid sequences of each of these expressed molecules are identical to one another. Thus, the nucleic acid molecule described herein may encode only one type of CAR.

The endodomain of a CAR may contain further domains. For example, the CAR may comprise domains that confer the ability on the CAR to provide a productive IL signal to the cell in which it is expressed in an antigen specific manner, without requiring exogenous IL to be administered. For example, it may comprise a domain comprising a STAT5 association motif, a JAK1 and/or JAK 2 binding motif and optionally a JAK 3 binding motif. In such an embodiment the endodomain may comprise one or more sequences from an endodomain of a cytokine receptor, for example an interleukin receptor (IL) receptor. Such CARs are described in WO2020/044055 (also incorporated herein by reference). Exemplary amino acid sequences derived from IL-2 receptor beta that include STAT5 association motif and JAK binding motifs are provided in SEQ ID NOs: 271-273. The inclusion of such domains confers the ability on the CAR to provide a productive IL signal to the cell in which it is expressed in an antigen-specific manner, without requiring exogenous IL to be administered. For example, IL-2 is important for the survival, proliferation and persistence of Treg cells, but IL-2 levels may frequently be low or impaired in patients needing treatment. A CAR may thus comprise a sequence corresponding to all or part of a β chain endodomain of an IL receptor or a variant thereof, such as the IL2 receptor, optionally in combination with the γ chain endodomain of an IL receptor or a variant thereof, such as the IL2 receptor.

### FOXP3

The nucleic acid molecule of the present invention may be designed to increase FOXP3 expression in cells (e.g., Tregs) by introducing into the cells a nucleotide sequence encoding FOXP3, which term is synonymous with the term “a FOXP3 polypeptide”. The nucleic acid molecule, and constructs and vectors containing it, thus provide a means for increasing FOXP3 in a cell, e.g., in a Treg or a CD4+ cell. Thus, the invention provides a cell comprising a nucleic acid molecule comprising a nucleotide sequence encoding a CAR and a nucleotide sequence encoding FOXP3, particularly a pluripotent cell (e.g. an iPSC), an HPC cell (e.g. expressing CD34), a CD4+ T cell or a Treg cell.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a cell (or population of cells) in comparison to a corresponding cell which has not been modified (or population of cells) by introduction of the nucleic acid molecule or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Optionally the cell is a Treg or the population of cells is a population of Tregs.

Suitably, the level of FOXP3 mRNA and/or protein in a modified cell (or a population of such cells) may be increased to at least 1.5-fold greater, 2-fold greater, or 5-fold greater than the level in a corresponding cell which has not been so modified (or population of such cells). Optionally the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3 (SEQ ID NO. 200), e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleotide sequence in the nucleic acid molecule described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24:209-26, incorporated herein by reference). As discussed in detail below, FOXP3 or a FOXP3 polypeptide includes functional fragments, variants, and isoforms thereof, e.g., of SEQ ID NO. 200.

A “functional fragment of FOXP3” may refer to a portion or region of a FOXP3 polypeptide or a polynucleotide (i.e., nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1 (6), pp. 665-675; Lopes, J. E., et al., 2006. The Journal of Immunology, 177 (5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 201 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, optionally at least 95% or at least 97% or at least 99% identical to a FOXP3 polypeptide or a polynucleotide encoding a FOXP3 polypeptide, e.g., to SEQ ID NO. 200. FOXP3 variants may have the same or similar activity to a wildtype FOXP3 polypeptide or polynucleotide, e.g., may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO. 200, for example S418E and/or S422A, as described in WO2019/241549 (incorporated herein by reference) and are set out in SEQ ID NO.s 202 to 204, which represent the aa418, aa422 and aa418 and aa422 mutants respectively.

Suitably, the FOXP3 polypeptide encoded by a nucleic acid molecule, construct or vector as described herein may comprise or consist of the polypeptide sequence of a human FOXP3, such as UniProtKB accession Q9BZS1 (SEQ ID NO: 200), or a functional fragment or variant thereof.

In some embodiments of the invention, the FOXP3 polypeptide comprises or consists of an amino acid sequence which is at least 70% identical to SEQ ID NO: 200 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 200 or a functional fragment thereof. In some embodiments, the FOXP3 polypeptide comprises or consists of SEQ ID NO: 200 or a functional fragment thereof.

In some embodiments, as discussed above, the FOXP3 polypeptide may comprise mutations at residues 418 and/or 422 of SEQ ID NO. 200, as set out in SEQ ID NO. 202, SEQ ID NO. 203, or SEQ ID NO. 204.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO. 201 or a functional variant thereof having at least 80, 85, 90, 95 or 99% identity thereto.

Suitably, the FOXP3 polypeptide may be a variant of SEQ ID NO: 200, for example a natural variant. Suitably, the FOXP3 polypeptide is an isoform of SEQ ID NO: 200. For example, the FOXP3 polypeptide may comprise a deletion of amino acid positions 72-106 relative to SEQ ID NO: 200. Alternatively, the FOXP3 polypeptide may comprise a deletion of amino acid positions 246-272 relative to SEQ ID NO: 200.

Suitably, the FOXP3 polypeptide comprises SEQ ID NO: 205 or a functional fragment thereof. SEQ ID NO: 205 represents an illustrative FOXP3 polypeptide.

Suitably the FOXP3 polypeptide comprises or consists of an amino acid sequence which is at least 70% identical to SEQ ID NO: 205 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 205 or a functional fragment thereof. In some embodiments, the FOXP3 polypeptide comprises or consists of SEQ ID NO: 205 or a functional fragment thereof.

Suitably, the FOXP3 polypeptide may be a variant of SEQ ID NO: 205, for example a natural variant. Suitably, the FOXP3 polypeptide is an isoform of SEQ ID NO: 205 or a functional fragment thereof. For example, the FOXP3 polypeptide may comprise a deletion of amino acid positions 72-106 relative to SEQ ID NO: 205. Alternatively, the FOXP3 polypeptide may comprise a deletion of amino acid positions 246-272 relative to SEQ ID NO: 205.

Suitably, the polynucleotide encoding a FOXP3 polypeptide comprises or consists of a nucleotide sequence set forth in SEQ ID NO: 206, which represents an illustrative FOXP3 nucleotide sequence.

In some embodiments of the invention, the polynucleotide encoding the FOXP3 polypeptide or variant comprises nucleotide sequence which is at least 70% identical to SEQ ID NO: 206 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 206 or a fragment thereof which encodes a functional FOXP3 polypeptide. In some embodiments of the invention, the polynucleotide encoding the FOXP3 polypeptide or variant comprises or consists of SEQ ID NO: 206 or a fragment thereof which encodes a functional FOXP3 polypeptide.

Suitably, the polynucleotide encoding a FOXP3 polypeptide comprises or consists of a polynucleotide sequence set forth in SEQ ID NO: 207, which represents another illustrative FOXP3 nucleotide.

In some embodiments of the invention, the polynucleotide encoding the FOXP3 polypeptide or variant comprises a nucleotide sequence which is at least 70% identical to SEQ ID NO: 207 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 207 or a fragment thereof which encodes a functional FOXP3 polypeptide. In some embodiments of the invention, the polynucleotide encoding the FOXP3 polypeptide or variant comprises or consists of SEQ ID NO: 207 or a fragment thereof which encodes a functional FOXP3 polypeptide.

A skilled person will appreciate that FOXP3 expression within a Treg may be increased indirectly by introducing a polynucleotide into the cell which encodes a protein which increases transcription and/or translation of FOXP3 or which increases the half life (e.g. by at least 10, 20, 30, 40, 50, 60, 70, 80 or 90%) or function of FOXP3 (e.g. determined by suppressive ability of a transduced Treg, measured as previously discussed). For example, it may be possible to introduce a polynucleotide into a Treg which increases transcription of endogenous FOXP3 by interacting with the endogenous FOXP3 promoter or non-coding sequences (CNS, e.g. CNS1, 2 or 3) which are found upstream of the coding region. Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

### Other Transgenes

In certain embodiments, the expression construct and/or the nucleic acid molecule of the invention does not comprise any further coding sequences (in addition to the sequences encoding the CAR and the FOXP3 polypeptide).

In alternative embodiments, the expression construct and/or the nucleic acid molecule of the invention comprises one or more further coding sequences (in addition to the sequences encoding the CAR and the FOXP3 polypeptide). In certain embodiments, the expression construct and/or the nucleic acid molecule of the invention comprises one further coding sequence(s).

The further coding sequence(s) may be selected from sequences which encode a molecule which increases the persistence of cells in which it is expressed, a safety switch polypeptide or a selectable marker.

The molecule which may be introduced into the cell or population of cells in order to improve persistence of the cells may provide a productive IL signal to the cell without requiring exogenous IL to be administered. Such IL signal may be constitutive or inducible. Exemplary technologies may, for example, involve the use of engineered or chimeric receptors that can transmit an IL signal without requiring exogenous IL to be administered. For example, inducible engineered receptors such as those described in WO 2018/111834, WO 2019/169290 and WO 2020/264039; or constitutive engineered receptors such as those described in WO 2018/038954, WO 2019/102207, WO 2019/053420, WO 2020/180694 and WO 2017/218850; chimeric cytokine receptors such as those described in WO 2020/183131, WO 2017/029512, WO 2012/138858, WO 2014/172584, WO 2017/068360, WO 2021/023987, WO 2020/180664 and WO 2020/044239; or engineered receptors having a tethered activation molecule such as those described in WO 2017/201432 and WO 2019/183389.

The safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the CAR, which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9. However, the suicide moiety may be, or comprise, an epitope which is recognised by a cell-deleting antibody or other binding molecule capable of eliciting deletion of the cell. In such an embodiment, the safety switch polypeptide is expressed on the surface of a cell.

The term “delete” as used herein in the context of cell deletion is synonymous with “remove” or “ablate” or “eliminate”. The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. Biosimilars for Rituximab are available and may be used. A person of skill in the art is readily able to use routine methods to prepare an antibody having the binding specificity of Rituximab using the available amino acid sequences therefor.

CAR-cells specific for antigen, which also express a safety switch polypeptide comprising this sequence can be selectively killed using the antibody Rituximab, or an antibody having the binding specificity of Rituximab. The safety switch polypeptide is expressed on the cell surface and when the expressed polypeptide is exposed to or contacted with Rituximab, or an antibody with the same binding specificity, death of the cell ensues.

Thus, Rituximab, or an antibody having the binding specificity thereof, may be provided for use in ACT in combination with a cell of the invention. The cell or nucleic acid or vector or construct for production of the cell and the Rituximab or equivalent antibody may be provided in a kit, or as a combination product.

For example, the suicide constructs of WO2013/153391 or WO2021/239812 (both incorporated herein by reference) may be used in a cell or cell population (e.g., Treg or Treg population) as described herein.

A further coding sequence may encode a selectable marker. Suitably selectable markers are well known in the art and include, but are not limited to, fluorescent proteins-such as GFP. Suitably, the selectable marker may be a fluorescent protein, for example GFP, YFP, RFP, tdTomato, dsRed, or variants thereof. In some embodiments the fluorescent protein is GFP or a GFP variant.

Suitably, the selectable marker/reporter domain may be a luciferase-based reporter, a PET reporter (e.g. Sodium lodide Symporter (NIS)), or a membrane protein (e.g. CD34, or Thy1.1).

The use of a selectable marker is advantageous as it allows cells (e.g., Tregs) in which an expression construct, nucleic acid molecule or vector of the present invention has been successfully introduced (such that the encoded CAR and FOXP3 are expressed) to be selected and isolated from a starting cell population using common methods, e.g., flow cytometry.

### Vector

A vector is a tool that allows or facilitates the transfer of an entity from one environment to another. As used herein, and by way of example, some vectors used in recombinant nucleic acid techniques allow entities, such as a segment of nucleic acid (e.g., a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g., in vitro transcribed mRNAs), chromosomes, artificial chromosomes and viruses. The vector may also be, for example, a naked nucleic acid (e.g., DNA). In its simplest form, the vector may itself be a nucleic acid molecule.

The vectors used herein may be, for example, plasmid, mRNA or virus vectors and may include a promoter (as described above) for the expression of a nucleic acid molecule/polynucleotide and optionally a regulator of the promoter.

In an embodiment the vector is a viral vector, for example a retroviral, e.g., a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal. Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g., act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g., operably linked to the promoter.

Vectors comprising the present expression constructs and/or nucleic acid molecules may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, optionally complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14:556) and combinations thereof.

Although the present expression constructs and nucleic acid molecules are designed to be used as single constructs, and this would be contained in a single vector, it is not precluded that they are introduced into a cell in conjunction with other vectors, for example encoding other polypeptides it may be desired also to introduce into the cell.

Engineered cells may be generated by introducing an expression construct, nucleic acid molecule, or vector as defined herein, by one of many means including transduction with a viral vector, and transfection with DNA or RNA.

### Cells

The cell may be an immune cell, or a precursor therefor. A precursor cell may be a progenitor cell. Representative immune cells thus include T-cells, in particular, cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, tolerogenic NK or NKT cells, dendritic cells, MDSC, neutrophils, and macrophages. Precursors of immune cells include pluripotent stem cells, e.g., induced PSC (iPSC), or more committed progenitors including multipotent stem cells (e.g. HPCs), or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL), helper T cell or a Treg cell.

In an embodiment the immune cell is a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

A T cell as used herein is a lymphocyte including any type of T cell, such as an alpha beta T cell (e.g., CD8 or CD4+), a gamma delta T cell, a memory T cell, a Treg cell.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs as disclosed herein may proliferate 5%, 10%, 20%, 30%, 40%, 50%, 60%, 70%, 90%, 95% or 99% less than the same Tconv cells cultured in the absence of the Tregs. For example, antigen-specific Tconv cells co-cultured with the present Tregs may proliferate 5%, 10%, 20%, 30%, 40%, 50%, 60%, 70%, 90%, 95% or 99% less than the same Tconv cells cultured in the presence of non-engineered Tregs. The cells comprising the nucleic acid, expression construct or vector as defined herein, e.g., Tregs, may have an increased suppressive activity as compared to non-engineered Tregs (e.g., an increased suppressive activity of at least 5, 10, 20, 30, 40, 50, 60, 70, 80 or 90%).

Antigen-specific Tconv cells co-cultured with the Tregs herein may express at least 10%, at least 20%, at least 30%, at least 40%, at least 50%, or at least 60% less effector cytokine than corresponding Tconv cells cultured in the absence of the Tregs (e.g., in the presence of non-engineered Tregs). The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural Tregs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38 (6), pp. 1654-1663).

Further suitable Tregs include, but are not limited to, Tr1 cells (which do not express Foxp3, and have high IL-10 production); CD8⁺FOXP3⁺ T cells; and γδ FOXP3⁺ T cells.

Different subpopulations of Tregs are known to exist, including naïve Tregs (CD45RA⁺FoxP3^{low}), effector/memory Tregs (CD45RA⁻FoxP3^{high}) and cytokine-producing Tregs (CD45RA-FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which optionally do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g., less than 50, 60, 70, 80 or 90% suppressive than naïve Tregs). Reference to expression levels herein may refer to mRNA or protein expression. Particularly, for cell surface markers such as CD45RA, CD25, CD4, CD45RO etc, expression may refer to cell surface expression, i.e., the amount or relative amount of a marker protein that is expressed on the cell surface. Expression levels may be determined by any known method of the art. For example, mRNA expression levels may be determined by Northern blotting/array analysis, and protein expression may be determined by Western blotting, or optionally by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g., a memory Treg cell). Alternatively viewed, a naïve Treg cell may express at least 2, 3, 4, 5, 10, 50 or 100-fold the amount of CD45RA as compared to a non-naïve Treg cell (e.g., a memory Treg cell). The level of expression of CD45RA can be readily determined by methods of the art, e.g., by flow cytometry using commercially available antibodies. Typically, non-naïve Treg cells do not express CD45RA or low levels of CD45RA.

Particularly, naïve Tregs may not express CD45RO, and may be considered to be CD45RO. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g., at least 10, 20, 30, 40, 50, 60, 70, 80 or 90% lower level of expression. Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, the cell, such as a Treg, is isolated from peripheral blood mononuclear cells (PBMCs) obtained from a subject. Suitably the subject from whom the PBMCs are obtained is a mammal, optionally a human. Suitably the cell is matched (e.g. HLA matched) or is autologous to the subject to whom the engineered cell is to be administered. Suitably, the subject to be treated is a mammal, optionally a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered cell is to be administered.

Suitably, the Treg is part of a population of cells. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. An expression construct, nucleic acid molecule or vector as described herein may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference).

As used herein, the term “conventional T cell” or Tcon or Tconv (used interchangeably herein) means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may be generated from a Tcon by introducing the nucleic acid which includes a sequence coding for FOXP3. Alternatively, the engineered Treg may be generated from a Tcon by in vitro culture of CD4+CD25-FOXP3-cells in the presence of IL-2 and TGF-β.

In another embodiment the target cell into which the nucleic acid molecule, expression construct or vector is introduced is not a cell intended for therapy. In an embodiment the cell is a production host cell. The cell may be for production of the expression construct or nucleic acid, e.g., cloning, or vector, or polypeptides.

The invention also provides a cell population comprising a cell as defined or described herein. It will be appreciated that a cell population may comprise both cells of the invention comprising a nucleic acid molecule, expression construct or vector as defined herein, and cells which do not comprise a nucleic acid molecule, expression construct or vector of the invention, e.g., untransduced or untransfected cells. Although in an embodiment, all the cells in a population may comprise a nucleic acid, expression construct or vector of the invention, cell populations having at least 10, 20, 30, 40, 50, 60, 70, 80, 90, 95 or 99% of cells comprising a nucleic acid, expression construct or vector of the invention are provided. Further, the population of cells may comprise more than one cell type, although in an embodiment, at least 10, 20, 30, 40, 50, 60, 70, 80, 90, 95 or 99% of the cells are of the same type. Particularly, a cell population may comprise at least 70, 80, 90, 95 or 99% of Tcells, more particularly Tregs. Additionally, at least 10, 20, 30, 40, 50, 60, 70, 80, 90, 95 or 99% of the Tcells, particularly Tregs may comprise a nucleic acid, expression construct or vector of the invention.

In particular, the invention provides a cell population comprising a plurality of cells comprising an expression construct or nucleic acid molecule of the invention.

### Methods of Making

The present cell may be made by: introducing to a cell (e.g. by transduction or transfection) the expression construct, nucleic acid molecule or vector as defined herein.

Suitable cells are discussed further above, but the cell may be from a sample isolated from a subject. The subject may be a donor subject, or a subject for therapy (i.e., the cell may be an autologous cell, or a donor cell, for introduction to another recipient, e.g., an allogeneic cell).

The cell may be generated by a method comprising the following steps:
(i) isolation of a cell-containing sample from a subject or provision of a cell-containing sample; and
(ii) introduction into (e.g., by transduction or transfection) the cell-containing sample of an expression construct, nucleic acid molecule or vector as defined herein, to provide a population of engineered cells.

A target cell-enriched sample may be isolated from, enriched, and/or generated from the cell-containing sample prior to and/or after step (ii) of the method. For example, isolation, enrichment and/or generation of Tregs (or other target cells) may be performed prior to and/or after step (ii) to isolate, enrich or generate a Treg-enriched sample. Isolation and/or enrichment from a cell-containing sample may be performed after step (ii) to enrich for cells and/or Tregs (or other target cells) comprising the expression construct, nucleic acid molecule or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching other target cells are known in the art.

Suitably, an engineered target cell may be generated by a method comprising the following steps:
(i) isolation of a target-cell enriched sample from a subject or provision of a target cell-enriched sample; and
(ii) introduction into (e.g., by transduction or transfection) the target cell-enriched sample of a nucleic acid, expression construct or vector as defined herein, to provide a population of engineered target cells.

The target cell may be a Treg cell, or precursor or a progenitor thereof.

An “engineered cell” means a cell which has been modified to comprise or express a polynucleotide which is not naturally encoded by the cell. Methods for engineering cells are known in the art and include, but are not limited to, genetic modification of cells e.g., by transduction such as retroviral or lentiviral transduction, transfection (such as transient transfection-DNA or RNA based) including lipofection, polyethylene glycol, calcium phosphate and electroporation, as discussed above. Any suitable method may be used to introduce an expression construct or nucleic acid into a cell. Non-viral technologies such as amphipathic cell penetrating peptides may be used to introduce nucleic acid. A cell may also be genetically modified e.g. using any known gene editing technique to insert an expression construct, nucleotide, polynucleotide or nucleic acid sequence as described herein into the genome, e.g. using CRISPR, Talens or Zn fingers.

Accordingly, the expression construct or nucleic acid molecule as described herein is not naturally expressed by a corresponding, unmodified cell. Indeed, the expression construct or nucleic acid molecule encoding the CAR is an artificial construct such that it could not occur or be expressed naturally. Suitably, an engineered cell is a cell which has been modified e.g., by transduction or by transfection. Suitably, an engineered cell is a cell which has been modified or whose genome has been modified e.g., by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign nucleic acid, e.g., DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing an expression construct or nucleic acid as described herein by one of many means including transduction with a viral vector, transfection with DNA or RNA. Cells may be activated and/or expanded prior to, or after, the introduction of an expression construct or nucleic acid as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g., Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

### Pharmaceutical Compositions and Therapeutic Uses

There is also provided a pharmaceutical composition comprising a cell or cell population as defined or described herein, or a vector as defined herein. The vector may be used for gene therapy. Thus, rather than administering a cell, a vector may be administered instead, to modify endogenous cells in the subject to express the introduced nucleic acid molecule. Vectors suitable for use in gene therapy are known in the art, and include viral vectors.

Thus, in a further aspect, the invention provides a cell, cell population or pharmaceutical composition as defined herein for use in therapy.

A pharmaceutical composition is a composition that comprises or consists of a therapeutically effective amount of a pharmaceutically active agent, i.e., the cell (e.g., Treg), cell population or vector. It optionally includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” it is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The cell, cell population or pharmaceutical composition as described herein can be administered parenterally, for example, intravenously or intrathecally, or they may be administered by infusion techniques. The cell, cell population or pharmaceutical composition may be administered in the form of a sterile aqueous solution which may contain other substances, for example, enough salts or glucose to make the solution isotonic with blood. The aqueous solution may be suitably buffered (optionally to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise cells in infusion media, for example sterile isotonic solution. The pharmaceutical composition may be enclosed in ampoules, disposable syringes or multiple dose vials made of glass or plastic.

The cell, cell population or pharmaceutical composition may be administered in a single or in multiple doses. Particularly, the cell, cell population or pharmaceutical composition may be administered in a single, one-off dose. The pharmaceutical composition may be formulated accordingly.

Depending upon the disease/condition and subject to be treated, as well as the route of administration, the cell, cell population or pharmaceutical composition may be administered at a specific stage of disease.

In type 1 diabetes, for example, pancreatic beta cells are destroyed such that they cannot produce insulin. Therefore, an optimum time to administer the cell, cell population or pharmaceutical composition of the invention may be at an early stage of the disease before all pancreatic beta cells have been destroyed, in order to maintain at least some functioning pancreatic beta cells (with residual pancreatic beta cell function) and maintain production of insulin. Particularly, at least 10, 20, 30, 40, 50, 60, 70, 80, 90 or 95% of pancreatic beta cells may be present at the time of administration of the cell, cell population or pharmaceutical composition described herein. Alternatively viewed, less than 5, 10, 20, 30, 40, 50, 60, 70, 80 or 90% of the pancreatic beta cells may have been destroyed before administration of the cell, cell population or pharmaceutical composition described herein.

The pharmaceutical composition may further comprise one or more active agents. The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18).

Depending upon the disease/condition and subject to be treated, as well as the route of administration, the cell, cell population or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for the cells herein, doses of 5×10⁷ to 3×10⁹ cells, or 10⁸ to 2×10⁹ cells per subject may be administered.

The cell may be appropriately modified for use in a pharmaceutical composition. For example, cells may be cryopreserved and thawed at an appropriate time, before being infused into a subject.

The invention further includes the use of kits comprising the cell, cell population and/or pharmaceutical composition herein. Optionally said kits are for use in the methods and uses as described herein, e.g., the therapeutic methods as described herein. Optionally said kits comprise instructions for use of the kit components.

The cell, cell population and pharmaceutical composition of the invention may find particular utility in the treatment of disorders associated with cells that express antigen (e.g., ENTPD3), or with disorders where antigen (e.g., ENTPD3) is localised at or near the site of disease, particularly disorders that would benefit from the immunosuppressive activity or target killing activity of the cells of the invention.

The cells, cell populations, compositions and vectors herein may be for use in treating, preventing or reducing the risk of a disease or condition in a subject, notably a disease or condition which may be treated by or with the CAR. The cells and compositions containing them are for adoptive cell therapy (ACT). Various conditions may be treated by administration of cells, including particularly Treg cells, expressing a CAR according to the present disclosure. As noted above, this may be conditions responsive to immunosuppression, and particularly the immunosuppressive effects of Tregs cells. The cells, cell populations, compositions and vectors described herein may thus be used for inducing, or achieving, immunosuppression in a subject. The Treg cells administered, or modified in vivo, may be targeted by expression of the CAR. Conditions suitable for such treatment include inducing tolerance to a transplant, treating and/or preventing graft-versus-host disease (GvHD), infectious, allergic, autoimmune or inflammatory diseases (e.g. type I diabetes), or more broadly a condition associated with any undesired or unwanted or deleterious immune response. Additionally, the cells, cell populations, compositions and vectors herein may be for use in promoting tissue repair and/or tissue regeneration.

Conditions to be treated or prevented include inflammation, or alternatively put, an inflammatory disease or a condition associated with or involving inflammation. Inflammation may be chronic or acute. Furthermore, the inflammation may be low-level or systemic inflammation. For example, the inflammation may be inflammation which occurs in the context of a metabolic disorder, for example metabolic syndrome, or in the context of insulin resistance, or type II diabetes or obesity and such like.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, bullous pemphigoid, sarcoidosis, ichthyosis, Graves ophthalmopathy, Addison's disease, Vitiligo, asthma, allergic asthma, acne vulgaris, chronic prostatitis, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “autoimmune disease” refers to a disease or condition in which a subject's immune system has an aberrant immune response against a substance that does not normally elicit an immune response in a healthy subject. Examples of autoimmune diseases include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenia purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Flemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenia purpura (TTP), Tolosa-Hunt syndrome, Transverse myelitis, Type 1 diabetes, Undifferentiated connective tissue disease (UCTD), Uveitis, Vasculitis, Vesiculobullous dermatosis, Vitiligo, or Wegener's granulomatosis (e., Granulomatosis with Polyangiitis (GPA).

The autoimmune or allergic disease may be selected from inflammatory skin diseases including psoriasis and dermatitis (e.g. atopic dermatitis); allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example Amylotrophic Lateral Sclerosis (ALS); neuroinflammatory disease; Chronic inflammatory demyelinating polyneuropathy (CIPD) and juvenile onset diabetes.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The term “target cell” refers to any cell expressing antigen (e.g., ENTPD3) to which the cell of the invention is to be directed to exert its therapeutic effect. In some embodiments, the target cell functions as a marker of a disease site, i.e. to attract the cells of the invention to provide an immunosuppressive effect. In some embodiments, the target cell is killed or abrogated by the cells of the invention. As noted above, in some embodiments, the target cell will be a pancreatic beta cell.

In particular, the disease or disorder to be treated may be type 1 diabetes. Other diseases or disorders that may be treated using the ENTPD3-specific CARs described herein include, for example, autoimmune pancreatitis, type 2 diabetes and insulinomas. Particularly the CARs may be expressed in cells having immunosuppressive function (e.g. CD4+ or CD8+ T-regulatory cells, tolerogenic NK or NKT cells, gamma-delta cells and immune regulatory 1 cells (Tr1) and other cells secreting immune modulatory cytokines such as IL-10, TGFbeta, IL-35 or amphiregulin) in order to treat type 1 diabetes, autoimmune pancreatitis or type 2 diabetes. The CARs may be expressed in cells having effector functions (e.g. T-effector cells, NK cells, NKT cells) in order to treat insulinomas.

Further, the CARs described herein may be used to prevent rejection of transplanted cells expressing antigen (e.g., ENTPD3) (e.g. beta cell replacements including allogeneic islet transplants, xenogeneic islet transplants and stem cell derived beta cells). Particularly, the CARs may be expressed in cells having immunosuppressive function (e.g. CD4+ or CD8+ T-regulatory cells, tolerogenic NK or NKT cells, gamma-delta cells and immune regulatory 1 cells (Tr1) and other cells secreting immune modulatory cytokines such as IL-10, TGFbeta, IL-35 or amphiregulin) in order to prevent rejection of transplanted cells expressing antigen (e.g., ENTPD3).

The engineered cells, e.g., Tregs, may be administered to a subject with a disease in order to lessen, reduce, or improve at least one symptom of disease such as hyperglycemia. The at least one symptom may be lessened, reduced, or improved by at least 10%, at least 20%, at least 30%, at least 40%, or at least 50%, or the at least one symptom may be completely alleviated.

The engineered cells, e.g., Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

As used herein “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immune suppressive drugs that a transplant recipient requires, or may enable the discontinuation of immunosuppressive drugs.

In one embodiment, the subject is a transplant recipient undergoing immunosuppression therapy. CAR-Tregs specific for HLA-A2 may be able to traffic to sites of HLA-A2 expression present on transplanted organs but not in the transplant recipient. The transplant may be a liver, kidney, heart, lung, pancreas, intestine, stomach, bone marrow, vascularized tissue graft, or skin transplant. For example the transplant may be a liver transplant.

In particular, the disease to be treated may be type 1 diabetes. As mentioned above, CAR-Tregs specific for ENTPD3 may be able to traffic to sites of ENTPD3 expression and control inflammation, via their bystander effect, to slow down the rate of pancreatic beta cell destruction.

Type 1 diabetes is a chronic autoimmune disease in which pancreatic beta cells, which are responsible for the production of insulin, are destroyed by the immune system. This is triggered by genetic and environmental factors. Destruction of beta cells reduces or eliminates production of insulin by the body and results in inflammation in the pancreatic islets. Insulin is a hormone required to regulate glucose levels in the bloodstream and, before treatment, subjects with type 1 diabetes will have excessively high blood sugar levels (hyperglycaemia). Type 1 diabetes is a serious and lifelong condition. Patients with type 1 diabetes currently need to closely monitor their blood glucose levels and take an appropriate dose insulin (e.g. by injection or pump). This therapy is not a cure and must be continuously administered. Over time, irregularities in blood sugar levels, including large fluctuations in glycaemic level, can result in long-term complications such as damage to the heart, eyes, feet and kidneys, as well as a reduction in life expectancy. It is noted that glycaemic control is particularly poor in younger subjects, particularly subjects between the ages of approximately 16 and 25 years and the therapies described herein may therefore particularly be useful for this population.

Type 1 diabetes is a continuum that progresses sequentially at variable but predictable rates through distinct identifiable stages prior to the onset of symptoms. This is described in more detail in Insel et al, Diabetes Care. 2015; 38 (10): 1964-1974. The ability to screen for risk and to stage type 1 diabetes prior to symptomatic type 1 diabetes provides an opportunity to intervene early to delay and ultimately prevent the onset of clinical symptoms.

Individuals at increased risk of developing type 1 diabetes can be identified by genetic screening. The HLA region on chromosome 6 accounts for about 30-50% of the genetic risk of type 1 diabetes, with the greatest association with HLA class II haplotypes RB1*0301-DQB1*0201 (DR3-DQ2) and DRB1*0401-DQB1*0302 (DR4-DQ8). The remaining genetic risk for type 1 diabetes can be attributed to approximately 50 non-HLA genes or loci identified by candidate gene and genome-wide association study approaches. The highest non-HLA genetic contribution arises from the INS, PTPN22, CTLA4 and IL2RA genes. Individuals who have been identified at increased risk of type 1 diabetes, but have not yet developed any signs of disease (i.e. pre-stage 1) may be prophylactically administered CAR-Tregs as described herein.

Stage 1 represents individuals who have developed two or more type 1 diabetes-associated islet autoantibodies (to insulin, GAD65, IA-2 and/or ZnT8) but have normal blood glucose levels.

Stage 2 represents individuals who have developed two or more type 1 diabetes-associated islet autoantibodies (to insulin, GAD65, IA-2 and/or ZnT8) but whose disease has progressed to the development of glucose intolerance, or dysglycemia, from loss of functional pancreatic beta cell mass. Dysglycemia may be defined as fasting blood glucose levels equal to or greater than 5.6 mmol/L, or 2 hour plasma glucose with a 75 g oral glucose tolerance test (OGTT) equal to or greater than 7.8 mmol/L, high glucose levels at intermediate time points on OGTT (30, 60, 90 minute levels equal to or greater than 11.1 mmol/L), and/or HbA1c levels equal to or greater than 5.7% (39 mmol/mol).

Stage 3 represents individuals who have typical clinical symptoms and signs of diabetes, which include, for example, polyuria, polydipsia, weight loss, fatigue and diabetic ketoacidosis (DKA).

The CAR-Tregs described herein may, for example, be used to treat subjects at stage 1, stage 2 and/or stage 3 of disease. Alternatively, the CAR-Tregs described herein may be used to treat a subject at risk of T1D, i.e., before Stage 1.

Where the subject is at stage 3 of disease, CAR-Treg treatment should begin as soon as possible to minimize pancreatic beta cell destruction and maximize residual pancreatic beta cell function. This may, for example, be determined by measuring blood insulin levels or blood C-peptide levels in the subject.

It may, for example, be a requirement that the subject has a minimum stimulated C-peptide of 0.2 pmol/mL, or for example a minimum stimulated C-peptide of 0.4 μmol/mL from a mixed meal tolerance test at the time of CAR-Treg administration. These subjects may be considered to have “recent-onset type 1 diabetes”.

For example, where the subject is at stage 3 of disease, CAR-Treg treatment may begin no more than about 24 weeks after the onset of symptoms or after diagnosis, for example no more than about 20 weeks or no more than about 16 weeks or no more than about 15 weeks or no more than about 14 weeks or no more than about 12 weeks or no more than about 8 weeks or no more than about 6 weeks after the onset of symptoms or after diagnosis. For example, CAR-Treg treatment may begin equal to or less than 100 days after the onset of symptoms or after diagnosis.

The subject may, for example, be at any age. For example, the subject may be under 30 years of age or under 25 years of age or under 20 years of age or under 18 years of age or under 16 years of age. Particularly, the subject may be from 8-30 years old, particularly from 8-25 years old, from 8-16 years old or from 16-25 years old.

Disease progression and pancreatic beta cell death may, for example, be monitored by a number of methods.

For example, imaging of pancreatic beta cells can be used to highlight beta cell integrity. The CAR-Tregs described herein may, for example, maintain or increase the number of pancreatic beta cells present in a subject after they are administered. This may, for example, be observed when the subject is receiving a reduced dose or no dose of exogenous insulin.

For example, insulin levels in a subject can be monitored. Normal fasting blood insulin levels (blood insulin levels after the subject has fasted (not eaten or drunk anything except water) for at least 8 hours) may be considered to be around 2 to 20 mIU/mL. The CAR-Tregs described herein may, for example, maintain or increase fasting blood insulin levels in a subject after they are administered to the subject, for example within the normal range for fasting blood insulin. This may, for example, be observed when the subject is receiving a reduced dose or no dose of exogenous insulin.

Insulin cell-free DNA (cfDNA) and unmethylated insulin may also be detected.

C-peptide levels in a subject can be monitored. The beta cells of the pancreas first produce a protein called “proinsulin”. Each proinsulin breaks down to one molecule of insulin and one molecule of C-peptide. Both are released when blood sugar levels are raised. Insulin and C-peptide are released in equal amounts but are broken down differently. Therefore, C-peptide can be employed as a surrogate marker of beta cell function. The liver breaks down insulin at a variable rate, while the kidneys break down C-peptide at a fairly steady rate. C-peptide may therefore be a more reliable measure of insulin production and beta cell function. Normal fasting (i.e. after fasting for at least 8 hours)C-peptide levels may be considered to be around 0.8 to 3.85 ng/ml. The CAR-Tregs described herein may, for example, maintain or increase fasting C-peptide levels in a subject after they are administered to the subject, for example within the normal range for fasting C-peptide. This may, for example, be observed when the subject is receiving a reduced dose or no dose of exogenous insulin.

Further, blood glucose levels in a subject can be monitored. Normal fasting blood glucose levels (i.e. after fasting for at least 8 hours). Normal fasting blood glucose levels may be considered to be around 3.9 to 6.9 mmol/L. Alternatively or additionally, an oral glucose tolerance test could be performed and blood glucose levels measured (e.g. using 75 g oral glucose). 2 hours following oral glucose, normal blood glucose levels may be considered to be less than around 7.8 mmol/L. At intermediate time points prior to the 2 hours (e.g. 30 minutes, 60 minutes, 90 minutes), normal blood glucose levels may be considered to be less than around 11.1 mmol/L. The CAR-Tregs described herein may, for example, maintain or reduce fasting blood glucose or OGTT blood glucose levels in a subject after they are administered to the subject, for example within the normal range for fasting blood glucose and/or OGTT blood glucose. This may, for example, be observed when the subject is receiving a reduced dose or no dose of exogenous insulin.

Haemoglobin (Hb) HbA1c levels in a subject can be monitored. HbA1c is created when glucose binds to haemoglobin and is a measure of average blood sugar levels over the preceding 2 to 3 months. Normal HbA1c values are considered to be approximately 4.0 to 5.6% (20-38 mmol/mol). The CAR-Tregs may, for example, maintain or reduce HbA1c levels in a subject after they are administered to the subject, for example within the normal range for HbA1c. This may, for example, be observed when the subject is receiving a reduced dose or no dose of exogenous insulin.

Suitably, the subject is a mammal. Suitably, the subject is a human.

Suitably, the cell may be an engineered Treg cell and the cell population may be a population of engineered Treg cells, which have been engineered to express a CAR as described herein.

Suitably, the CAR may comprise an antigen binding domain which is capable of specifically binding to ENTPD3, i.e., the antigen is ENTPD3.

A method for treating a disease or condition relates to the therapeutic use of the cells herein. In this respect, the cells may be administered to a subject having an existing disease or condition in order to lessen, reduce or improve at least one symptom associated with the disease or condition and/or to slow down, reduce or block the progression of the disease.

Suitably, inducing tolerance to a transplant, treating and/or preventing graft-versus-host disease (GvHD), and treating and/or preventing an infectious, allergic, autoimmune or inflammatory disease may refer to administering an effective amount of the cells (e.g., Tregs) such that the amount of existing medication (e.g. exogenous insulin) that a subject with said disease requires is reduced, or may enable the discontinuation of the subject's existing medication.

Preventing a disease or condition relates to the prophylactic use of the cells herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition (e.g. pre-stage 1 type 1 diabetes).

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to affect a measurable improvement of a particular disease, disorder, or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the cell, cell population or pharmaceutic compositions are outweighed by the therapeutically beneficial effects.

The terms “subject”, “patient” and “individual” are used interchangeably herein and refer to a mammal, optionally a human. In particular, the terms subject, patient and individual refer to a human having a disease or disorder as defined herein in need of treatment.

In some embodiments of the invention, the patient may be subjected to other treatments prior to, contemporaneously with, or after the treatments of the present invention. For instance, in some embodiments, the patient may be treated with other procedures for the treatment of symptoms associated with the disease or disorder.

The medical use of or method herein may involve the steps of:
(i) isolating a cell-containing sample or providing a cell-containing sample;
(ii) introducing a nucleic acid molecule, expression construct or a vector as defined herein to the cell; and
(iii) administering the cells from (ii) to a subject.

The cell may be a Treg as defined herein. An enriched Treg population may be isolated and/or generated from the cell containing sample prior to, and/or after, step (ii) of the method. For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide, and/or the vector as described herein.

Suitably, the cell may be autologous. Suitably, the cell may be allogenic.

Suitably, the cell (e.g., the engineered Treg) may be administered in combination with one or more other therapeutic agents, such as lympho-depletive agents (e.g., as discussed above). The engineered cell, e.g., Treg, may be administered simultaneously with or sequentially with (i.e., prior to or after) the one or more other therapeutic agents.

Cells, e.g., Tregs, may be activated and/or expanded prior to, or after, the introduction of an expression construct nucleic acid molecule as described herein, for example by treatment with an anti-CD3 monoclonal antibody or both anti-CD3 and anti-CD28 monoclonal antibodies. Expansion protocols are discussed above.

The cell, e.g., Tregs, may be washed after each step of the method, in particular after expansion.

The population of engineered cells, e.g., Treg cells may be further enriched by any method known to those of skill in the art, for example by FACS or magnetic bead sorting.

The steps of the method of production may be performed in a closed and sterile cell culture system.

### Further Uses

The invention may also provide a method for increasing the stability and/or suppressive function of a cell comprising the step of introducing a nucleic acid molecule, an expression construct or vector as provided herein into the cell. An increase in suppressive function can be measured as discussed above, for example by co-culturing activated antigen-specific Tconv cells with cells of the invention, and for example measuring the levels the cytokines produced by the Tconv cells. An increase in suppressive function may be an increase of at least 10, 20, 30, 40, 50, 60, 70, 80, or 90% as compared to a non-engineered Treg.

An increase in stability of a cell, e.g., a Treg as defined herein, refers to an increase in the persistence or survival of those cells or to an increase in the proportion of cells retaining a Treg phenotype over a time period (e.g., to cells retaining Treg markers such as FOXP3 and Helios) as compared to a non-engineered Treg.

An increase in stability may be an increase in stability of at least 10, 20, 30, 40, 50, 60, 70, 80 or 90%, and may be measured by techniques known in the art, e.g., staining of Treg cell markers within a population of cells, and analysis by FACS.

The invention also provides a method for increasing the expression of a CAR and/or FOXP3 polypeptide in a cell, the method comprising the step of introducing an expression construct, nucleic acid molecule or vector as provided herein to the cell. An increase in CAR and/or FOXP3 can be measured as discussed herein, for example by flow cytometry. The increase in expression may be an increase in % transduction, for example an increase of at least 5% (percentage points), 10% (percentage points), 15% (percentage points), 20% (percentage points) or 25% (percentage points). The increase in expression may be an increase in MFI, for example an increase of at least 10, 20, 30, 40, 50, 60, 70, 80, or 90%. The increase may be as compared to the expression levels in a cell which an expression construct, nucleic acid molecule or vector has been introduced, wherein the expression construct comprises a PGK promoter operably linked to a first nucleotide sequence encoding the CAR and second nucleotide sequence encoding the FOXP3 polypeptide, where the second nucleotide sequence is located upstream of the first nucleotide sequence. Alternatively, the increase may be as compared to the expression levels in a cell which an expression construct, nucleic acid molecule or vector has been introduced, wherein the nucleic acid molecule comprises an alternative mammalian promoter (e.g. short EF1a or full length EF1a) operably linked to a first nucleotide sequence encoding the CAR and second nucleotide sequence encoding the FOXP3 polypeptide, where the first nucleotide sequence is located upstream of the second nucleotide sequence.

The invention also provides the use of an expression construct, nucleic acid molecule or vector as provided herein to increase expansion of a cell in which it is expressed, particularly to increase expansion of a Treg in which it is expressed. The invention further provides a method for increasing the expansion of a cell, the method comprising the step of introducing an expression construct, nucleic acid molecule or vector as provided herein to the cell, particularly wherein the cell is a Treg. The increase in expansion may be relative to an expression construct, nucleic acid molecule or vector that is identical except that it uses an alternative promoter in place of the PGK promoter. The alternative promoter may, for example, be a SFFV promoter (e.g. SEQ ID NO: 215). The fold expansion may, for example, increase by at least about 20% or at least about 30% or at least about 40% or at least about 50% or at least about 60% or at least about 70% or at least about 80% or at least about 90% or at least about 100% relative to fold expansion obtained using the expression construct, nucleic acid molecule or vector that is identical except that it uses an alternative promoter in place of the PGK promoter (e.g. SFFV promoter). The fold expansion may, for example, increase by up to about 200% relative to fold expansion obtained using the expression construct, nucleic acid molecule or vector that is identical except that it uses an alternative promoter in place of the PGK promoter (e.g. SFFV promoter).

The invention also provides the use of a CAR-Treg to reduce the rate or prevent death of pancreatic beta cells, for example in a subject having or at risk of developing type 1 diabetes, particularly recent-onset type 1 diabetes. This may, for example, be determined by measuring number of pancreatic beta cells, blood insulin levels and/or blood C-peptide levels in a subject. Any maintenance or improvement in number of pancreatic beta cells, blood insulin levels and/or blood C-peptide levels may be considered to be reducing the rate or preventing death of pancreatic beta cells. This may, for example, be observed when the subject is receiving a reduced dose or no dose of exogenous insulin.

The invention thus also provides the use of a CAR-Treg to maintain or increase fasting blood insulin levels and/or fasting blood C-peptide levels in a subject, for example in a subject having or at risk of developing type 1 diabetes, particularly recent-onset type 1 diabetes. This may, for example, be observed when the subject is receiving a reduced dose or no dose of exogenous insulin.

The invention further provides the use of a CAR-Treg to reduce or prevent hyperglycemia, for example in a subject having or at risk of developing type 1 diabetes, particularly recent-onset type 1 diabetes. This may, for example, be determined by measuring fasting blood glucose and/or HbA1c levels in a subject. Any maintenance or improvement in blood glucose levels and/or HbA1c levels in a subject may be considered to be reducing or preventing hyperglycaemia in a subject. This may, for example, be observed when the subject is receiving a reduced dose or no dose of exogenous insulin.

The invention thus also provides the use of a CAR-Treg to maintain or decrease fasting blood glucose and/or HbA1c levels in a subject, for example in a subject having or at risk of developing type 1 diabetes, particularly recent-onset type 1 diabetes. This may, for example, be observed when the subject is receiving a reduced dose or no dose of exogenous insulin.

### Numbered Paragraphs

The following numbered paragraphs defined particular embodiments of the present invention:
1. An expression construct comprising a PGK promoter operably linked to:
   (i) a first nucleotide sequence encoding a chimeric antigen receptor (CAR); and
   (ii) a second nucleotide sequence encoding a FOXP3 polypeptide;
   wherein the first nucleotide sequence is located upstream of the second nucleotide sequence.
2. The expression construct of paragraph 1, wherein the expression construct does not comprise any other coding nucleotide sequence.
3. The expression construct of any preceding paragraph, wherein the first and second nucleotide sequences are separated by the presence of a self-cleavage sequence and/or an internal ribosome entry site (IRES).
4. The expression construct of paragraph 3, wherein the self-cleavage sequence is a 2A sequence, optionally selected from P2A, T2A, E2A and F2A.
5. The expression construct of any preceding paragraph, wherein the PGK promoter comprises or consists of the sequence of SEQ ID NO: 195 or a functional variant or functional fragment thereof.
6. The expression construct of any preceding paragraph, wherein the PGK promoter is a truncated human PGK promoter or a functional variant having at least 70% identity thereto, optionally wherein the truncated human PGK promoter has the sequence of SEQ ID NO: 196, SEQ ID NO: 197, SEQ ID NO: 198 or SEQ ID NO: 199.
7. The expression construct of any preceding paragraph, wherein the CAR is a human CAR.
8. The expression construct of any preceding paragraph, wherein the CAR comprises an exodomain comprising an antigen recognition domain.
9. The expression construct of paragraph 8, wherein the antigen recognition domain specifically binds to ENTPD3.
10. The expression construct of any preceding paragraph, wherein the CAR comprises:
   a. an exodomain comprising an antigen recognition domain;
   b. a transmembrane domain; and
   c. an endodomain comprising an intracellular signalling domain.
11. The expression construct of paragraph 10, wherein the CAR further comprises a hinge domain and/or one or more co-stimulatory domains.
12. The expression construct of paragraph 11, wherein the CAR comprises a CD8α or CH2CH3 hinge domain, a CD28, CD8α or CH2CH3 transmembrane domain, a CD28 co-stimulatory domain, and a CD3zeta signalling domain, wherein when the hinge domain is CD8α, the transmembrane domain is CD8α, and when the hinge domain is CH2CH3, the transmembrane domain is CD28 or CH2CH3.
13. The expression construct of any of paragraphs 8 to 12, wherein the antigen recognition domain is an antibody, an antibody fragment, or derived from an antibody, optionally wherein the antigen recognition domain is a single chain antibody (scFv).
14. The expression construct of any of paragraphs 8 to 13, wherein the antigen recognition domain comprises:
   i. VH CDR1, 2 and 3 sequences set forth in SEQ ID NOs: 7, 8 and 9 respectively and VL CDR1, 2 and 3 sequences set forth in SEQ ID NOs: 10, 11 and 12 respectively;
   ii. VH CDR1, 2 and 3 sequences set forth in SEQ ID NOs: 19, 20 and 21 respectively and VL CDR1, 2 and 3 sequences set forth in SEQ ID NOs: 22, 23 and 24 respectively;
   iii. VH CDR1, 2 and 3 sequences set forth in SEQ ID NOs: 73, 74 and 75 respectively and VL CDR1, 2 and 3 sequences set forth in SEQ ID NOs: 76, 77 and 78 respectively;
   iv. VH CDR1, 2 and 3 sequences set forth in SEQ ID NOs: 25, 26 and 27 respectively and VL CDR1, 2 and 3 sequences set forth in SEQ ID NOs: 28, 29 and 30 respectively;
   v. VH CDR1, 2 and 3 sequences set forth in SEQ ID NOs: 37, 38 and 39 respectively and VL CDR1, 2 and 3 sequences set forth in SEQ ID NOs: 40, 41 and 42 respectively;
   vi. VH CDR1, 2 and 3 sequences set forth in SEQ ID NOs: 1, 2 and 3 respectively and VL CDR1, 2 and 3 sequences set forth in SEQ ID NOs: 4, 5 and 6 respectively;
   vii. VH CDR1, 2 and 3 sequences set forth in SEQ ID NOs: 13, 14 and 15 respectively and VL CDR1, 2 and 3 sequences set forth in SEQ ID NOs: 16, 17 and 18 respectively;
   viii. VH CDR1, 2 and 3 sequences set forth in SEQ ID NOs: 31, 32 and 33 respectively and VL CDR1, 2 and 3 sequences set forth in SEQ ID NOs: 34, 35 and 36 respectively;
   ix. VH CDR1, 2 and 3 sequences set forth in SEQ ID NOs: 43, 44 and 45 respectively and VL CDR1, 2 and 3 sequences set forth in SEQ ID NOs: 46, 47 and 48 respectively;
   x. VH CDR1, 2 and 3 sequences set forth in SEQ ID NOs: 49, 50 and 51 respectively and VL CDR1, 2 and 3 sequences set forth in SEQ ID NOs: 52, 53 and 54 respectively;
   xi. VH CDR1, 2 and 3 sequences set forth in SEQ ID NOs: 55, 56 and 57 respectively and VL CDR1, 2 and 3 sequences set forth in SEQ ID NOs: 58, 59 and 60 respectively;
   xii. VH CDR1, 2 and 3 sequences set forth in SEQ ID NOs: 61, 62 and 63 respectively and VL CDR1, 2 and 3 sequences set forth in SEQ ID NOs: 64, 65 and 66 respectively;
   xiii. VH CDR1, 2 and 3 sequences set forth in SEQ ID NOs: 67, 68 and 69 respectively and VL CDR1, 2 and 3 sequences set forth in SEQ ID NOs: 70, 71 and 72 respectively; or
   xiv. VH CDR1, 2 and 3 sequences set forth in SEQ ID NOs: 79, 80 and 81 respectively and VL CDR1, 2 and 3 sequences set forth in SEQ ID NOs: 82, 83 and 84 respectively;
   wherein one or more of said CDR sequences of (i) to (xiv) may optionally comprise 1 to 3 amino acid modifications relative to an aforementioned CDR sequence, particularly wherein one or more of said CDR sequences may optionally be modified by substitution, addition or deletion of 1 to 3 amino acids.
15. The expression construct of any of paragraphs 8 to 14, wherein the antigen recognition domain comprises:
   i. a VH domain comprising the sequence set forth in SEQ ID NO: 87, or a sequence having at least 70% sequence identity thereto, and a VL domain comprising the sequence as set forth in SEQ ID NO: 88, or a sequence having at least 70% identity thereto;
   ii. a VH domain comprising the sequence set forth in SEQ ID NO: 91, or a sequence having at least 70% sequence identity thereto, and a VL domain comprising the sequence as set forth in SEQ ID NO: 92, or a sequence having at least 70% identity thereto;
   iii. a VH domain comprising the sequence set forth in SEQ ID NO: 109, or a sequence having at least 70% sequence identity thereto, and a VL domain comprising the sequence as set forth in SEQ ID NO: 110, or a sequence having at least 70% identity thereto;
   iv. a VH domain comprising the sequence set forth in SEQ ID NO: 93, or a sequence having at least 70% sequence identity thereto, and a VL domain comprising the sequence as set forth in SEQ ID NO: 94, or a sequence having at least 70% identity thereto;
   v. a VH domain comprising the sequence set forth in SEQ ID NO: 97, or a sequence having at least 70% sequence identity thereto, and a VL domain comprising the sequence as set forth in SEQ ID NO: 98, or a sequence having at least 70% identity thereto;
   vi. a VH domain comprising the sequence set forth in SEQ ID NO: 85, or a sequence having at least 70% sequence identity thereto, and a VL domain comprising the sequence as set forth in SEQ ID NO: 86, or a sequence having at least 70% identity thereto;
   vii. a VH domain comprising the sequence set forth in SEQ ID NO: 89, or a sequence having at least 70% sequence identity thereto, and a VL domain comprising the sequence as set forth in SEQ ID NO: 90, or a sequence having at least 70% identity thereto;
   viii. a VH domain comprising the sequence set forth in SEQ ID NO: 95, or a sequence having at least 70% sequence identity thereto, and a VL domain comprising the sequence as set forth in SEQ ID NO: 96, or a sequence having at least 70% identity thereto;
   ix. a VH domain comprising the sequence set forth in SEQ ID NO: 99, or a sequence having at least 70% sequence identity thereto, and a VL domain comprising the sequence as set forth in SEQ ID NO: 100, or a sequence having at least 70% identity thereto;
   x. a VH domain comprising the sequence set forth in SEQ ID NO: 101, or a sequence having at least 70% sequence identity thereto, and a VL domain comprising the sequence as set forth in SEQ ID NO: 102, or a sequence having at least 70% identity thereto;
   xi. a VH domain comprising the sequence set forth in SEQ ID NO: 103, or a sequence having at least 70% sequence identity thereto, and a VL domain comprising the sequence as set forth in SEQ ID NO: 104, or a sequence having at least 70% identity thereto;
   xii. a VH domain comprising the sequence set forth in SEQ ID NO: 105, or a sequence having at least 70% sequence identity thereto, and a VL domain comprising the sequence as set forth in SEQ ID NO: 106, or a sequence having at least 70% identity thereto;
   xiii. a VH domain comprising the sequence set forth in SEQ ID NO: 107, or a sequence having at least 70% sequence identity thereto, and a VL domain comprising the sequence as set forth in SEQ ID NO: 108, or a sequence having at least 70% identity thereto; or
   xiv. a VH domain comprising the sequence set forth in SEQ ID NO: 111, or a sequence having at least 70% sequence identity thereto, and a VL domain comprising the sequence as set forth in SEQ ID NO: 112, or a sequence having at least 70% identity thereto.
16. The expression construct of any of paragraphs 8 to 15, wherein the antigen recognition domain comprises or consists of:
   i. the sequence set forth in SEQ ID NO: 114 or a sequence having at least 70% sequence identity thereto;
   ii. the sequence set forth in SEQ ID NO: 116 or a sequence having at least 70% sequence identity thereto;
   iii. the sequence set forth in SEQ ID NO: 125 or a sequence having at least 70% sequence identity thereto;
   iv. the sequence set forth in SEQ ID NO: 117 or a sequence having at least 70% sequence identity thereto;
   v. the sequence set forth in SEQ ID NO: 119 or a sequence having at least 70% sequence identity thereto;
   vi. the sequence set forth in SEQ ID NO: 113 or a sequence having at least 70% sequence identity thereto;
   vii. the sequence set forth in SEQ ID NO: 115 or a sequence having at least 70% sequence identity thereto;
   viii. the sequence set forth in SEQ ID NO: 118 or a sequence having at least 70% sequence identity thereto;
   ix. the sequence set forth in SEQ ID NO: 120 or a sequence having at least 70% sequence identity thereto;
   x. the sequence set forth in SEQ ID NO: 121 or a sequence having at least 70% sequence identity thereto;
   xi. the sequence set forth in SEQ ID NO: 122 or a sequence having at least 70% sequence identity thereto;
   xii. the sequence set forth in SEQ ID NO: 123 or a sequence having at least 70% sequence identity thereto;
   xiii. the sequence set forth in SEQ ID NO: 124 or a sequence having at least 70% sequence identity thereto; or
   xiv, the sequence set forth in SEQ ID NO: 126 or a sequence having at least 70% sequence identity thereto.
17. The expression construct of any preceding paragraph, wherein the CAR comprises or consists of the sequence of any one of SEQ ID NOs: 127 to 152 or a functional variant thereof having at least 70% sequence identity thereto, particularly wherein the CAR comprises or consists of the sequence of SEQ ID NO: 128 or 130 or a functional variant having at least 70% sequence identity thereto.
18. The expression construct of any preceding paragraph, wherein the FOXP3 polypeptide has the sequence of SEQ ID NO: 200, SEQ ID NO: 201, SEQ ID NO: 202, SEQ ID NO: 203, SEQ ID NO: 204, SEQ ID NO: 205 or a variant having at least 70% identity thereto, or the second nucleotide sequence encoding the FOXP3 polypeptide has the sequence of SEQ ID NO: 206, SEQ ID NO: 207 or a sequence having at least 80% identity thereto.
19. The expression construct of any preceding paragraph, wherein the expression construct comprises the sequence of SEQ ID NO: 214 or a sequence having at least 70% identity thereto.
20. A nucleic acid molecule comprising an expression construct of any preceding paragraph.
21. The nucleic acid molecule of paragraph 20, wherein the nucleic acid molecule does not comprise any other coding nucleotide sequence.
22. A vector comprising the expression construct or nucleic acid molecule of any preceding paragraph.
23. The vector of paragraph 22, wherein the vector is a plasmid or a viral vector, optionally a lentiviral vector or gamma-retroviral vector.
24. A cell comprising the expression construct of any of paragraphs 1 to 19 or the nucleic acid molecule of paragraph 20 or 21 or the vector of paragraph 22 or 23.
25. The cell of paragraph 24, wherein the cell is a production host cell.
26. The cell of paragraph 24, wherein the cell is an immune cell or a progenitor or precursor thereof, for example a T cell or precursor thereof, or a stem cell.
27. The cell of paragraph 24 or 26, wherein the cell is a regulatory T cell (Treg) or a precursor thereof, or an iPSC cell.
28. A cell population comprising a cell of any of paragraphs 24, 26 or 27 or a plurality of cells of any of paragraphs 24, 26 or 27, optionally a plurality of T cells of paragraph 26 or a plurality of Tregs of paragraph 27.
29. A pharmaceutical composition comprising a vector of paragraph 22 or 23, a cell of any of paragraphs 24, 26 or 27, or a cell population of paragraph 28.
30. The cell of any of paragraphs 24, 26 or 27, the cell population of paragraph 28 or the pharmaceutical composition of paragraph 29, for use in therapy.
31. The cell, cell population or pharmaceutical composition for use of paragraph 30, wherein the therapy is adoptive cell transfer therapy.
32. The cell of any of paragraphs 24, 26 or 27, the cell population of paragraph 28 or the pharmaceutical composition of paragraph 29, for use in inducing tolerance to a transplant, treating and/or preventing graft-versus-host disease (GvHD), treating or preventing an infectious, allergic, autoimmune or inflammatory disease, or for use in inducing immunosuppression, or for use in promoting tissue repair and/or tissue regeneration, particularly wherein the cell is a Treg.
33. The cell, cell population or pharmaceutical composition for use of paragraph 32, wherein the autoimmune or inflammatory disease is type 1 diabetes.
34. A method of making a cell of any of paragraphs 24 to 27, which comprises the step of introducing into the cell the expression construct of any of paragraphs 1 to 19 or the nucleic acid molecule of paragraph 20 or 21 or the vector of paragraph 22 or 23.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

### Materials & Methods

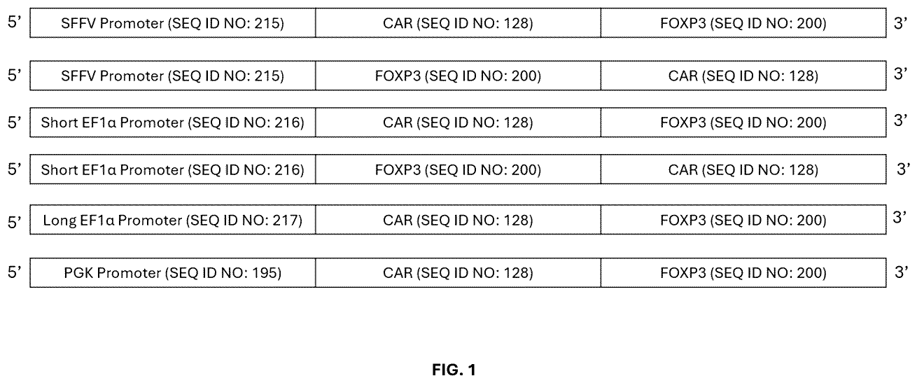

The constructs shown in FIG. 1 were designed and inserts were transferred into a lentiviral backbone by PCR cloning.

### Lentiviral Production:

Suspension HEK293T/17SF cells were seeded in 6 well plates and were transfected using transfection reagents with the helper plasmids, viral envelope and the DNA construct/plasmid of interest. After 48 h the lentiviral supernatant was harvested and filtered.

Infectious titre was determined by transducing SUP-T1 cell line with serial dilutions of vector. 3 days later, cells are harvested and stained for live/dead and CAR using anti-G4S-linked antibody (Cell Signaling Technology, Massachusetts, USA). CAR expression is determined using a flow cytometer and analysed to calculate an infectious titer (TU/mL) using the following equation: Titre (TU/mL)=[(% transduced cells/100)*Number of cells transduced]/sample volume*Dilution factor.

### Generation of Tregs Expressing the CAR and FOXP3

Regulatory T cells were purified and FACS sorted as CD4+ CD25+ CD127-cells from healthy donors. Cells were activated using Human T-Activator CD3/CD28 Dynabeads™ (ThermoFisher Scientific) in X-Vivo medium (Lonza) in the presence of Interleukin-2 (1000 IU/ml). After 48 hours of activation, cells were transduced with lentiviral particles comprising the constructs shown in FIG. 1. Cells were further expanded and at day 15 cells were harvested and counted. Transduction efficiency was assessed at day 15 post transduction by detecting CAR expressing using anti-G4S linker antibody (Cell Signaling Technology, Massachusetts, USA) and FOXP3 expression (Transcription Factor Staining Buffer Set, ThermoFisher Scientific).

### Results

The results are shown in the table below.

| | Infectious Titre | Treg transduction at |
|---|---|---|
| | (TU/mL) | day 15 (%) |
| SFFV-CAR-FOXP3 | 1.73E+09 | 40 |
| SFFV-FOXP3-CAR | 1.16E+09 | 22 |
| Short EF1α-CAR-FOXP3 | 1.91E+08 | 20 |
| Short EF1α-FOXP3-CAR | 6.64E+07 | 15 |
| Long EF1α-CAR-FOXP3 | 1.26E+08 | 18 |
| PGK-CAR-FOXP3 | 7.50E+08 | 45 |

### Conclusions

Constructs having the transgene order ‘CAR-FOXP3’ perform better than constructs having the transgene order ‘FOXP3-CAR’.

The construct having the PGK promoter performed better than the constructs including alternative mammalian promoters (short and long EF1a).

### Example 1A

### Materials & Methods

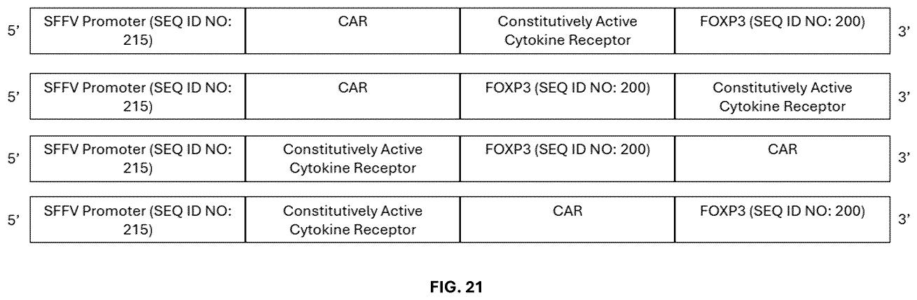

The constructs shown in FIG. 21 were designed and inserts were transferred into a lentiviral backbone by PCR cloning. The CAR includes a different binder and is directed to a different target compared to the CAR used in Examples 1, 2 and 3 herein. The CAR and constitutively active cytokine receptor (CACR) in each of the constructs shown in FIG. 21 is the same.

### Lentiviral Production:

Suspension HEK293T/17 SF cells were cultured in BalanCD media and seeded in shaker flasks. Transfection reagents were brought to room temperature and were mixed with DNA construct/plasmid of interest, helper plasmids and viral envelope. The DNA suspension was added to the cells and after 48 h the viral supernatant was harvested, filtered and concentrated.

Infectious titre was determined by transducing SUP-T1 cell line with serial 5-fold dilutions of vector (so D1=neat vector, D2=5-fold dilution, D3=25-fold dilution etc). 3 days later, cells are harvested and surface stained for LIVE/DEAD fixable dead cell stain (ThermoFisher Scientific, Massachusetts, USA), CAR using anti-G4S-linked antibody (Cell Signaling Technology, Massachusetts, USA) and anti-CACR. Washed cells were then fixed and permeabilised using FoxP3/Transcription Factor Staining Buffer Set (ThermoFisher Scientific) and anti-human FoxP3 antibody (BD Biosciences, New Jersey, USA) was used to intracellularly stain for FoxP3. CAR expression is determined using a flow cytometer and analysed to calculate an infectious titer (TU/mL) using the following equation: Titre (TU/mL)=[(% transduced cells/100)*Number of cells transduced]/sample volume*Dilution factor.

### Results

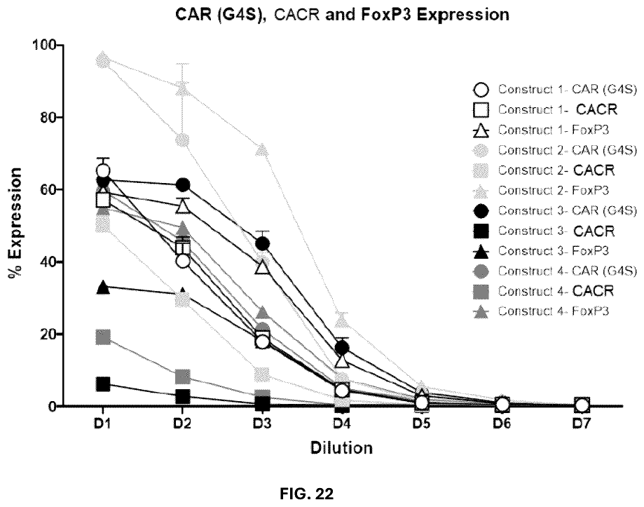

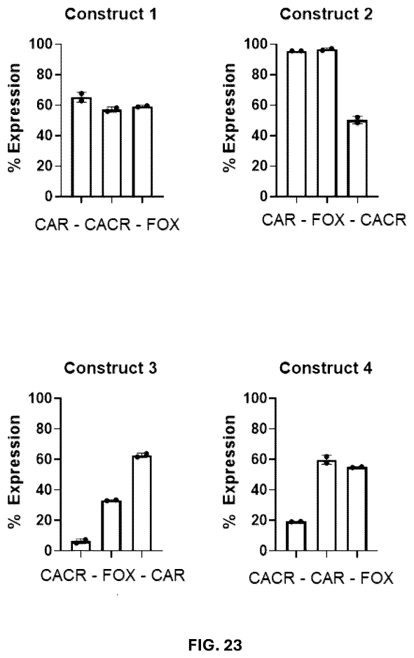

The results of the serial SUP-T1 dilutions are shown in FIGS. 22 and 23. The constructs with the gene order CAR-FOXP3 provided higher transduction efficiency than the construct with the gene order FOXP3-CAR.

### Example 1B

### Materials & Methods

The constructs shown in FIG. 21 were designed and inserts were transferred into a lentiviral backbone as described above.

### Treg Isolation Protocol:

Leukopaks were used to derive Treg populations. Leukopak-derived PBMCs are subjected to CD25 positive selection followed by a CD4 enrichment via negative selection using Human CD25 Positive Selection Cocktail and Human CD4+ T Cell Enrichment Cocktail (Stemcell Technologies), respectively. Cells of interest were separated using a magnet. CD127high Depletion Cocktail (Stemcell Technologies) were used to further separate the CD4+CD25+CD127−/low cell populations and cells of interest were separated using a magnet. Cell fractions were stained with flow cytometry antibodies anti-CD4 VioBlue (M-T466, Miltenyi), anti-CD25 PE (3G10, Miltenyi), anti-CD127 APC (MB15-18C9, Miltenyi), anti-CD45RA FITC (T6D11, Mitenyi) and LD 7AAD (BioLegend) before FACS sorting. CD4+CD25+CD127−/low CD45RA+ T cells (CD45RA+ Tregs) were sorted and used.

### Treg Transduction, Culture Media and Expansion:

Tregs were cultured in X-VIVO 15 media (Lonza) supplemented with 5% Human AB serum (heat-inactivated; Merck), activated with Human T-Activator CD3/CD28 Dynabeads™ (ThermoFisher Scientific) and supplemented with of 300 IU/ml Interleukin-2 (IL-2) (Proleukin, Clinigen Healthcare).

Non-tissue culture-treated 24-well plates were prepared by coating with Retronectin (Takahara-bio-Otsu, Japan). After 48 hours of activation, cells were transduced with lentiviral particles comprising the constructs shown in FIG. 1. Cells cultures were spinoculated and media exchange performed on alternate days. Tregs were cultured in X-VIVO 15 media supplemented with 5% Human AB serum and IL-2 (Proleukin, Clinigen Healthcare) and were re-fed every 2 to 3 days. A second round of stimulation may be performed to promote further expansion of Treg cells before using cells At day 14 cells were harvested and counted. Transduction efficiency was assessed at day 14 post transduction by detecting CAR expression using anti-G4S linker antibody (Cell Signaling Technology, Massachusetts, USA), FOXP3 expression (FoxP3/Transcription Factor Staining Buffer Set, ThermoFisher Scientific) and anti-CACR.

### Results

The results are shown in the table below (n=4 donors).

| | % CAR+ cells | % CACR+ cells | % FOXP3+ cells |
|---|---|---|---|
| MOCK | 0.15 | 0.84 | 86.40 |
| CAR-FOXP3-CACR | 31.43 | 24.44 | 89.88 |
| CAR-CACR-FOXP3 | 18.04 | 19.47 | 86.49 |
| CACR-FOXP3-CAR | 8.22 | 18.39 | 87.86 |
| CACR-CAR-FOXP3 | 20.60 | 23.08 | 88.97 |

The constructs with the gene order CAR-FOXP3 provided higher transduction efficiency than the construct with the gene order FOXP3-CAR.

### Example 2

### Materials & Methods

The constructs ‘SFFV-CAR-FOXP3’ and ‘PGK-CAR-FOXP3’ shown in FIG. 1 were designed and inserts were transferred into a lentiviral backbone by PCR cloning.

### Lentiviral Production:

Suspension HEK293T/17SF cells were seeded in 6 well plates and were transfected using transfection reagents with the helper plasmids, viral envelope and the DNA construct/plasmid of interest. After 48 h the lentiviral supernatant was harvested and filtered.

Infectious titre was determined by transducing SUP-T1 cell line with serial dilutions of vector. 3 days later, cells are harvested and stained for live/dead and CAR using anti-G4S-linked antibody (Cell Signaling Technology, Massachusetts, USA). CAR expression is determined using a flow cytometer and analysed to calculate an infectious titer (TU/mL) using the following equation: Titre (TU/mL)=[(% transduced cells/100)*Number of cells transduced]/sample volume*Dilution factor.

### Generation of Tregs Expressing the CAR and FOXP3

Regulatory T cells were purified and FACS sorted as CD4+CD25+CD127-cells from healthy donors. Cells were activated using Human T-Activator CD3/CD28 Dynabeads™ (ThermoFisher Scientific) in X-Vivo medium (Lonza) in the presence of Interleukin-2 (1000 IU/ml). After 48 hours of activation, cells were transduced with lentiviral particles comprising the constructs shown in FIG. 1. Cells were further expanded and at day 15 cells were harvested and counted.

Transduction efficiency was assessed at day 15 post transduction by detecting CAR expressing using anti-G4S linker antibody (Cell Signaling Technology, Massachusetts, USA) and FOXP3 expression (Transcription Factor Staining Buffer Set, ThermoFisher Scientific).

Vector copy number was determined by measuring WPRE (from the viral construct) in comparison to a housekeeping gene. Genomic DNA from transduced cells was isolated and prepared for qPCR along with standard controls. The quantity of WPRE and housekeeping gene are calculated using the standard controls and VCN per transduced cell was calculated as follows: VCN per transduced cell=[WPRE quantity mean/housekeeping quantity mean]/% transduced cells*100.

### Treg Activation Assay

For analysis of CAR-dependent Treg activation, rested Tregs were cultured in the presence of 1) RT-4 cells (which naturally express ENTPD3) at a ratio of 1:1 or 5:1 or 10:1 (Tregs: cell line); 2) HEK cells expressing ENTPD3 at a ratio of 5:1 Tregs: cell line; or 3) immobilised ENTPD3 protein at 0.5 ug/ml, 1 ug/ml, 2 ug/ml or 5 ug/ml. Here, 0.1*106 Treg were cultured. As a negative control, Tregs were cultured in the absence of a stimulus or in the presence of wildtype HEK cells at a ratio of 5:1 Tregs: cell line. As a positive control, Tregs were cultured in the presence of anti-CD3/CD28 activation beads.

After 24 h cells were harvested and stained with anti-CD4 and anti-CD69 antibodies. Cells were acquired on a flow cytometer and percentages of CD69 up-regulation after stimulation were calculated.

### Treg Proliferation Assay

To assess the proliferative capacity of Tregs in response to antigen, rested Tregs labelled with CellTrace Violet dye (ThermoFisher Scientific) were cultured in the presence of: 1) immobilised ENTPD3 protein at 1 ug/ml, 2 ug/ml or 5 ug/ml, 2) HEK293 expressing ENTPD3 at ratios of 5:1 (Tregs: cell line), or 3) RT-4 cells at a ratio of 10:1, 5:1 or 1:1 (Tregs: cell line). As a negative control, Tregs were cultured in media alone or in the presence of wildtype HEK cells at a ratio of 5:1 (Tregs: cell line). As a positive control, Tregs were cultured in the presence of anti-CD3/CD28 activation beads. Six days after assay set-up, cells were stained with anti-CD4 and anti-G4S linker antibodies and acquired by flow cytometry. The fold change of proliferation for each condition was calculated by dividing the percentage proliferation of that condition with its relevant control.

### Treg Suppression Assay

To assess the ability of Tregs to suppress effector T cell activation, Teff cells were labeled with CellTrace Violet dye (ThermoFisher Scientific). Labeled Teff cells were then co-cultured with different concentrations of rested Tregs (ratios Treg: Teff of 2:1, 1:1, 1:2, 1:4, 1:8, 1:16, 1:32, 0:1). The cells were activated using either anti-CD3/CD28 beads, wildtype B cells or B cells expressing ENTPD3 (for CAR-specific activation). Five days after assay set-up cells were harvested and analyzed by flow cytometry. CellTrace Violet dye dilution is used as a surrogate marker for Teff cell proliferation.

The Tregs were either untransduced (mock) or transduced with the ‘PGK-CAR-FOXP3’ or ‘SFFV-CAR-FOXP3’ construct.

### IL-2 Production by Teffs

The ability of SFFV and PGK to drive FOXP3 expression was assessed by determining the ability of the two constructs to inhibit production of IL-2 by Teffs transduced with the ‘SFFV-CAR-FOXP3’ and ‘PGK-CAR-FOXP3’ constructs shown in FIG. 1.

For the generation of Teff CAR-T cells, CD4 T cells were isolated from PBMCs by magnetic separation and transduced with the ‘SFFV-CAR-FOXP3’ and ‘PGK-CAR-FOXP3’ constructs shown in FIG. 1. An activation assay was set up with the Teff CAR-T cells with ENTPD3 peptide (CAR specific activation), anti-CD3/CD28 beads or media alone. Twenty-four hours later, the Teff CAR-T cells and supernatant were harvested for IL-2 assessment.

For the assessment of IL-2 secretion by Teffs, IL-2 was detected by standard Intracellular Flow Cytometry and supernatants were analysed by ELISA (Ella, BioTechne).

### Results

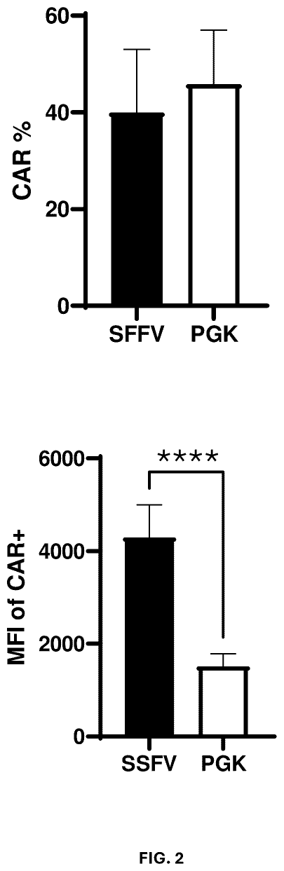

The results are shown in FIGS. 2 to 7 and in the table below. FIG. 2 shows the % of cells expressing CAR (top plot) and the mean fluorescence intensity (MFI) of CAR+ cells (bottom plot). The % of cells expressing CAR is similar for Tregs transduced with the ‘SFFV-CAR-FOXP3’ construct (n=10) and the ‘PGK-CAR-FOXP3’ construct (n=7). However, the MFI of CAR+ cells is significantly higher for Tregs transduced with the ‘SFFV-CAR-FOXP3’ construct compared to the ‘PGK-CAR-FOXP3’ construct.

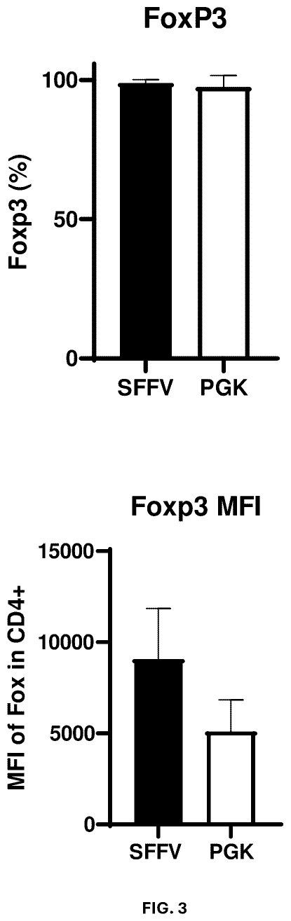

FIG. 3 the % of cells expressing FOXP3 (top plot) and the mean fluorescence intensity (MFI) of FOXP3 in CD4+ cells (bottom plot). The % of cells expressing FOXP3 is similar for Tregs transduced with the ‘SFFV-CAR-FOXP3’ construct (n=10) and the ‘PGK-CAR-FOXP3’ construct (n=7). However, the MFI of FOXP3 is significantly higher for Tregs transduced with the ‘SFFV-CAR-FOXP3’ construct compared to the ‘PGK-CAR-FOXP3’ construct.

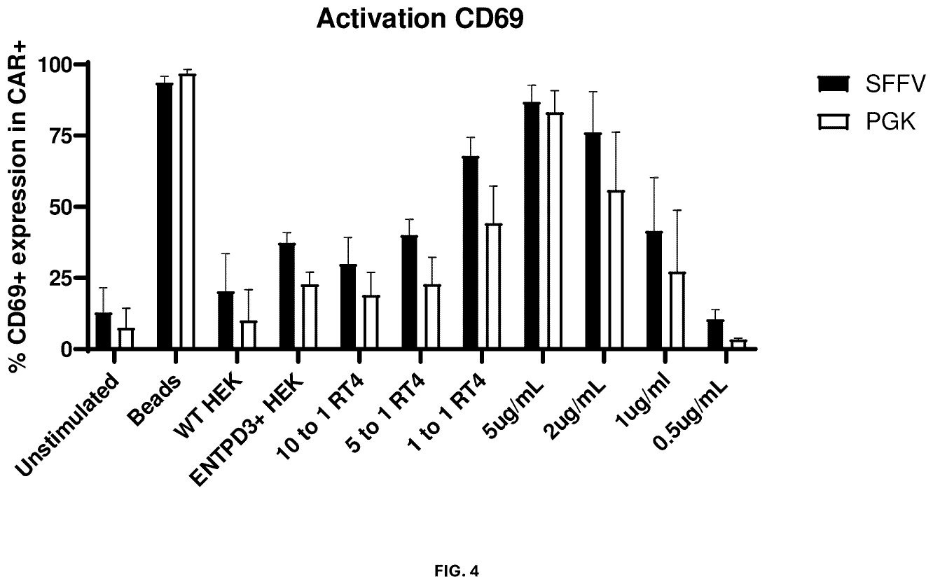

FIG. 4 shows the results of the activation assay, particularly % of cells expressing the activation marker CD69 (n=7 for all conditions except n=2 for 0.5 ug/ml ENTPD3 peptide condition and n=3 for RT4 and 1 ug/ml ENTPD3 peptide conditions). Antigen-specific activation is observed with both the ‘SFFV-CAR-FOXP3’ and ‘PGK-CAR-FOXP3’ constructs (ENTPD+HEK, RT4 and ENTPD3 peptide conditions). Activation is observed to be slightly higher in cells transduced with the ‘SFFV-CAR-FOXP3’ construct compared to ‘PGK-CAR-FOXP3’, although background activation is higher in cells transduced with the ‘SFFV-CAR-FOXP3’ construct (see unstimulated and WT HEK conditions).

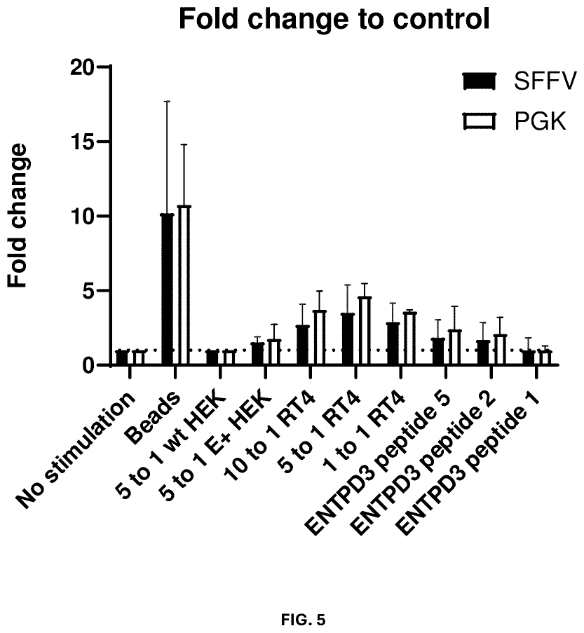

FIG. 5 shows the results of the proliferation assay (n=5-6 for all conditions except n=2 for RT4 conditions). ENTPD3+HEK condition was normalized to WT HEK condition. All other conditions were normalized to the unstimulated condition. The level of antigen-specific CAR-Treg proliferation upon stimulation with ENTPD3 was similar for cells transduced with the ‘SFFV-CAR-FOXP3’ and ‘PGK-CAR-FOXP3’ constructs.

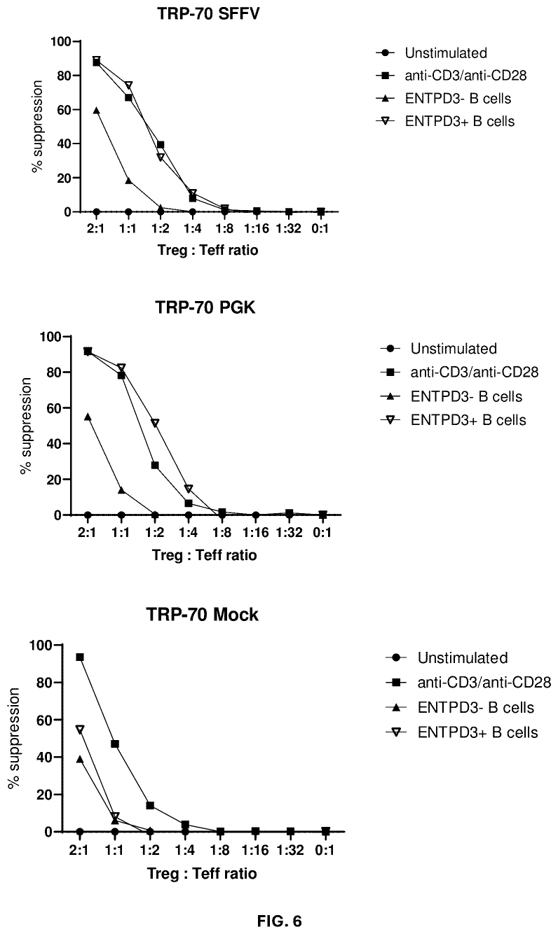

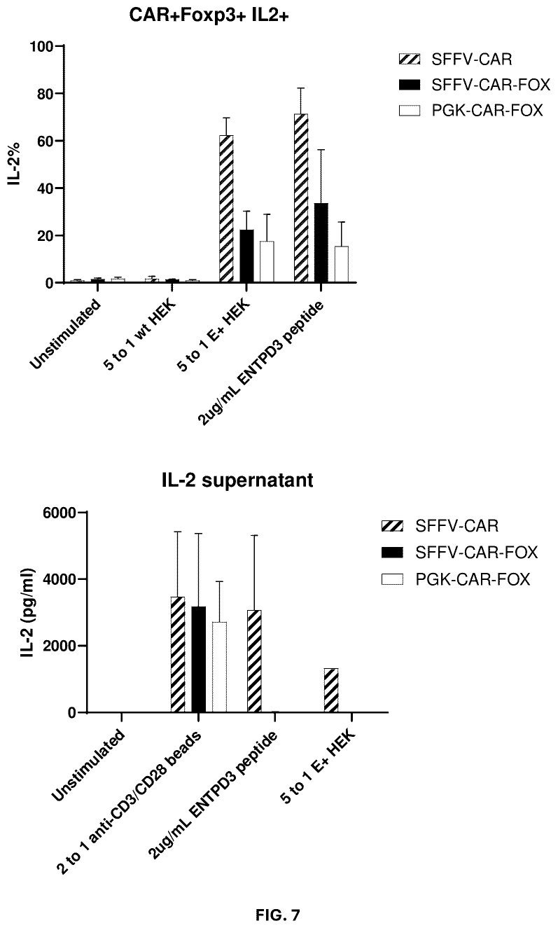

FIG. 6 shows the results of the suppression assay. Antigen-specific suppression was observed for Tregs transduced with both the ‘SFFV-CAR-FOXP3’ and ‘PGK-CAR-FOXP3’ constructs. FIG. 7 shows the results of the Teff IL-2 production assay (flow cytometry results on the left and supernatant results on right). There was similar inhibition of IL-2 production driven by the ‘SFFV-CAR-FOXP3’ and ‘PGK-CAR-FOXP3’ constructs.

| | SFFV-CAR-FOXP3 | PGK-CAR-FOXP2 |
|---|---|---|
| Infectious Titre (TU/mL) | 1.3 × 10⁹ | 7.5 × 10⁸ (first batch) |
| | | 3.3 × 10⁹ (second batch) |
| Treg transduction at day 15 | 40 | 45 |
| (%) | | |
| CAR MFI at day 15 | 4299 | 1510 |
| Vector Copy Number | 5 | ≤5 |
| Antigen-specific activation | 2.4 fold | 3.4 fold |
| (ENTPD3+ HEK cells vs WT | | |
| HEK cells) | | |
| Repression of IL-2 production | 200 fold less | 200 fold less |
| by Teffs relative to non-FOXP3 | IL-2 (pg/ml) | IL-2 (pg/ml) |
| control | | |
| Antigen-specific proliferation | 2.5 fold | 2.5 fold |
| (ENTPD3+ HEK cells vs WT | | |
| HEK cells) | | |

### Conclusions

Tregs transduced with the construct including the mammalian PGK promoter provided a similar CAR-dependent (antigen-specific) response compared to Tregs transduced with the construct including the viral SFFV promoter. This was surprising given that Tregs transduced with the construct including the PGK promoter had a lower CAR MFI than Tregs transduced with the construct including the SFFV promoter.

### Example 3

The constructs ‘SFFV-CAR-FOXP3’ and ‘PGK-CAR-FOXP3’ shown in FIG. 1 and used in Example 2 were used to generate the data presented in Example 3.

### Materials & Methods

Lentivirus and Tregs expressing the CAR and FOXP3 were generated as described in Example 2 above.

Expansion fold was calculated by dividing the absolute number of viable cells at the end of expansion (day 15) by the absolute number of viable cells at the beginning of expansion (day 0).

### Treg Phenotyping

Following Treg expansion, the Tregs were cryopreserved and thawed. Washed cells were then stained with LIVE/DEAD™ Fixable Aqua—Dead Cell Stain (Thermofisher) in PBS for 20 minutes at room temperature. Washed cells were then stained with anti-CCR7 BV421 (G043H7; BioLegend) in Brilliant Stain Buffer Plus at 37 degrees C. for 20 mins. Washed cells were then stained with anti-CD4 (SK3; BD Bioscience), anti-CD226 (11A8; BD Bioscience), anti-CD62L (DREG-56; BD Bioscience), anti-CD45RO (UCHL1; BD Bioscience), anti-OX-40 (Ber-ACT35; BioLegend), anti-CD45RA (HI100; BioLegend), anti-CD70 (Ki-24; BD Bioscience), anti-QBEND (QBEND/10; ThermoFisher), anti-CD39 (A1; BioLegend), anti-TIGIT (A15153G; BioLegend), anti-CD25 (M-A251; BioLegend), anti-CD27 (O323; BioLegend), anti-HLA-DR (L243; BioLegend), anti-IL1R1 (Polyclonal; Bio-Techne), anti-CTLA-4 (BNI3; BioLegend) an at 4 degrees C. for 30 mins. For intracellular staining of cells, cells were fixed and permeabilized using FOXP3/Transcription Factor Staining Buffer Set (Thermo Fisher Scientific, UK) according to the manufacturer's instructions. Permeabilised cells were stained with the anti-FOXP3 (206D; BioLegend), anti-HELIOS (22F6; BioLegend), anti-Ki-67 (Ki-67; BioLegend) in permeabilization buffer. Cells were washed twice before acquisition on a flow cytometer. Marker expression was analysed using FlowJo software.

### Treg Activation Assay

For analysis of CAR-dependent Treg activation, rested Tregs were cultured in the presence of 1) HEK cells expressing ENTPD3 at a ratio of 4:1 Tregs: cell line; or 2) EndoC-βH5 cells (human pancreatic beta cells that are functionally close to primary adult beta cells) at a ratio of 4:1 Tregs: cell line. Here, 0.1*106 Treg were cultured. As a negative control, Tregs were cultured in the absence of a stimulus or in the presence of wildtype HEK cells at a ratio of 5:1 Tregs: cell line. As a positive control, Tregs were cultured in the presence of anti-CD3/CD28 activation beads.

After 24 h cells were harvested and stained with anti-CD4, anti-CD69, anti-CD137 and anti-GARP antibodies. Cells were acquired on a flow cytometer and percentages of CD69, CD137 and GARP up-regulation after stimulation were calculated.

### Treg Suppression Assay

To assess the ability of Tregs to suppress effector T cell activation, Teff cells were labeled with CellTrace Violet dye (ThermoFisher Scientific). Labeled Teff cells were then co-cultured alone or with different concentrations of rested Tregs (ratios Treg: Teff of 1:1, 1:2, 1:4, 1:8, 1:16, 1:32, 1:64). The cells were activated using either anti-CD3/CD28 beads, wildtype B cells or B cells expressing ENTPD3 (for CAR-specific activation). Five days after assay set-up cells were harvested and analyzed by flow cytometry. CellTrace Violet dye dilution is used as a surrogate marker for Teff cell proliferation. Percentage suppression was calculated by normalizing proliferation of Teff cells in the presence of Tregs to Teff cells alone for each of the indicated ratios, % suppression of T cell proliferation was plotted and area under the curve calculated.

The Tregs were either untransduced (mock) or transduced with the ‘PGK-CAR-FOXP3’ or ‘SFFV-CAR-FOXP3’ construct.

### Depletion Assay

For analysis of CAR-dependent modulation of B cells (via CD80/CD86 depletion), Tregs were co-cultured with wildtype B cells or B cells expressing ENTPD3 at Treg: B cell ratios 10:1 to 1:4. Four days after assay set-up cells were harvested and analyzed by flow cytometry. CD80 and CD86 expression in the Treg conditions were normalized to the B cell alone control. % suppression of CD80 and CD86 was plotted and area under the curve calculated.

The Tregs were either untransduced (mock) or transduced with the ‘PGK-CAR-FOXP3’ or ‘SFFV-CAR-FOXP3’ construct.

### Results

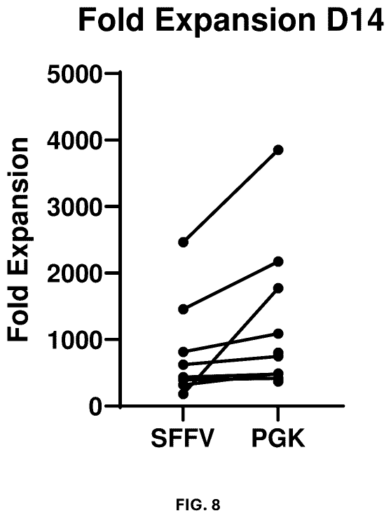

Cells obtained from each human donor were split and transduced with either the PGK-CAR-FOXP3 construct or the SFFV-CAR-FOXP3 construct. FIG. 8 shows expansion fold of cells transduced with the PGK-CAR-FOXP3 and cells transduced with the SFFV-CAR-FOXP3 construct across the different donors. Cells transduced with the PGK construct generally resulted in greater expansion fold compared to cells transduced with the SFFV construct.

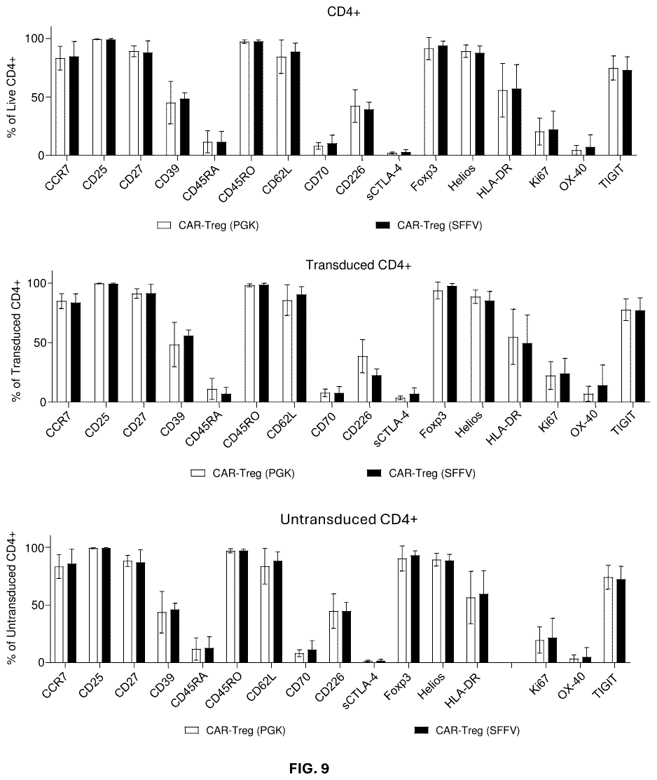

FIG. 9 shows the results of the Treg phenotype analysis. There were minimal phenotypic differences between Tregs transduced with the PGK construct and Tregs transduced with the SFFV construct.

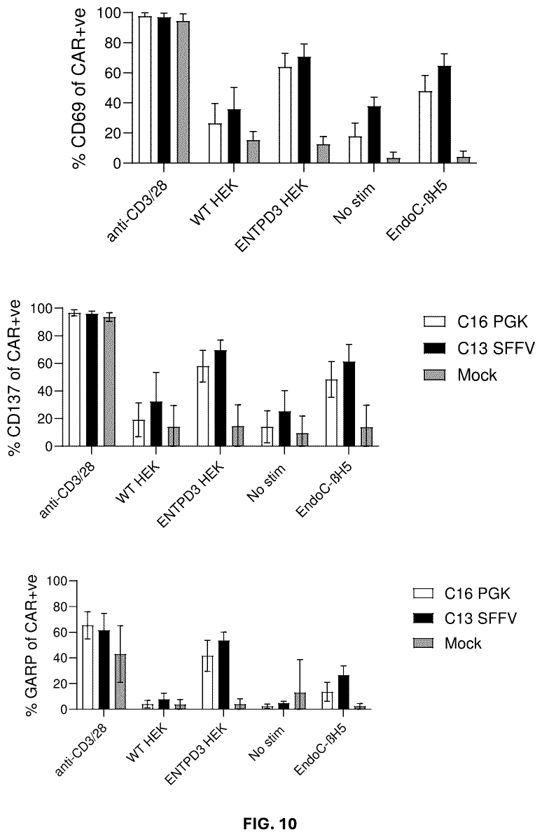

FIG. 10 shows the results of the activation assay, particularly % of cells expressing the activation markers CD69, CD137 and GARP (n=5). Antigen-specific activation is observed with both the ‘SFFV-CAR-FOXP3’ and ‘PGK-CAR-FOXP3’ constructs (ENTPD+HEK and EndoC-βH5 conditions). Activation is observed to be slightly higher in cells transduced with the ‘SFFV-CAR-FOXP3’ construct compared to ‘PGK-CAR-FOXP3’, although background activation is higher in cells transduced with the ‘SFFV-CAR-FOXP3’ construct (see unstimulated and WT HEK conditions). The EndoC-βH5 condition shows activation in response to physiological levels of antigen.

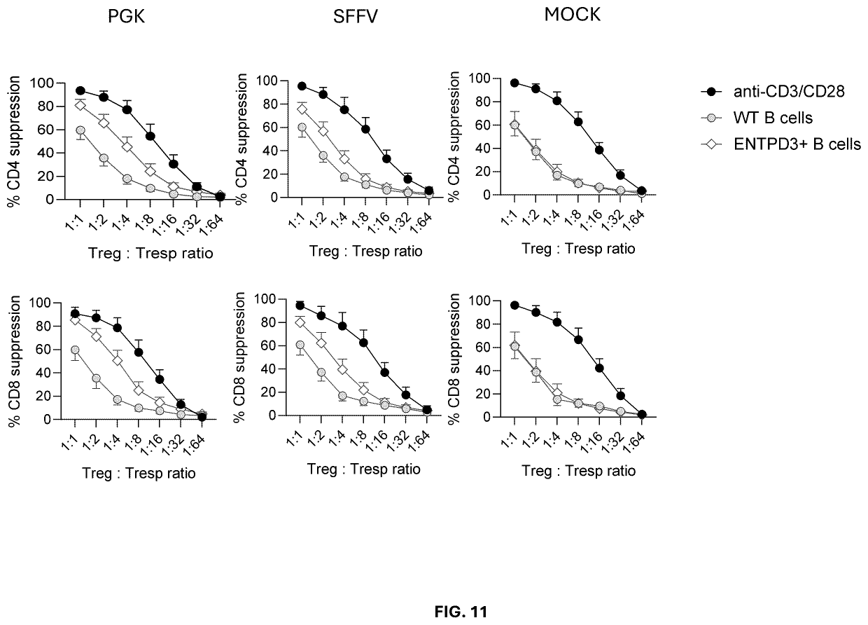

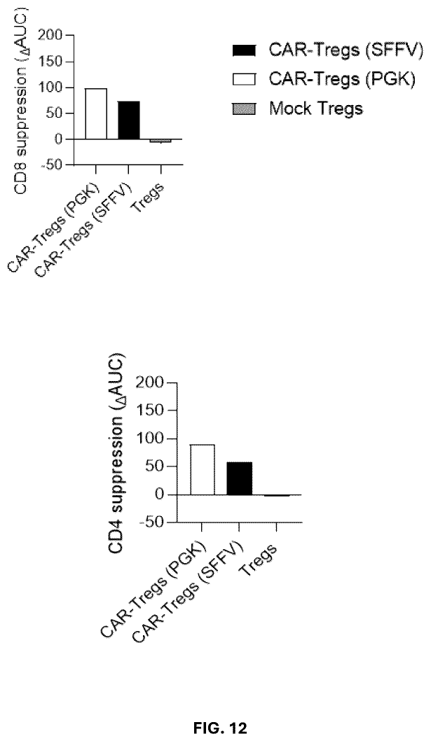

FIGS. 11 and 12 show the results of the suppression assay (n=8). Antigen-specific suppression was observed for Tregs transduced with both the ‘SFFV-CAR-FOXP3’ and ‘PGK-CAR-FOXP3’ constructs. Suppression by CAR-Tregs transduced with the SFFV construct was consistently lower than suppression by CAR-Tregs transduced with the PGK construct despite similar levels of polyclonal response (CD3/CD28 bead activation).

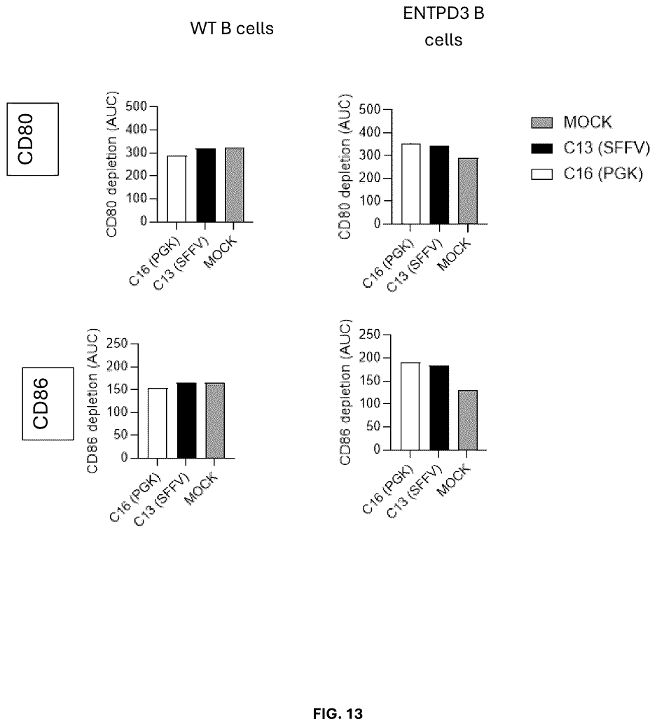

FIG. 13 shows the results of the depletion assay (n=8). When activated via the CAR (ENTPD3 B cell condition), depletion of CD80 and CD86 is higher by Tregs transduced with the PGK construct compared to Tregs transduced with the SFFV construct and untransduced (mock) Tregs.

### Conclusions

Tregs transduced with the construct including the mammalian PGK promoter provided improved cell expansion, CAR-dependent activation, suppression and APC modulation (CD80/CD86 depletion) compared to Tregs transduced with the construct including the SFFV promoter. This was surprising given that Tregs transduced with the construct including the PGK promoter had a lower CAR MFI than Tregs transduced with the construct including the SFFV promoter.

### Example 4

### Materials & Methods

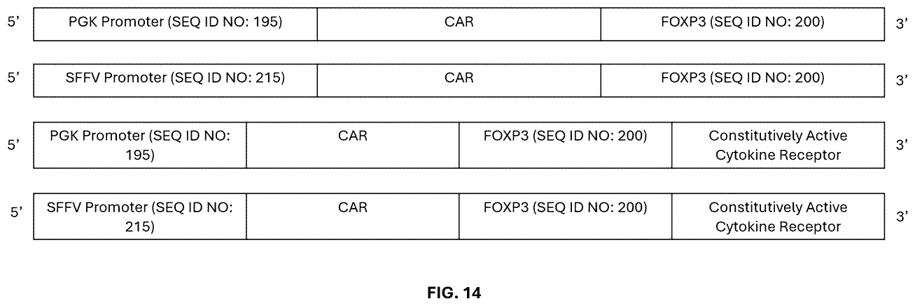

The constructs shown in FIG. 14 were designed and inserts were transferred into a lentiviral backbone by PCR cloning. The bicistronic constructs include a CAR and FOXP3. The tricistronic constructs include a CAR, FOXP3 and a constitutively active cytokine receptor (CACR). The CAR includes a different binder and is directed to a different target compared to the CAR used in Examples 1, 2 and 3 above. The CAR and CACR in each of the constructs shown in FIG. 14 is the same.

### Lentiviral Production:

Suspension HEK293T/17SF cells were seeded in shaker flasks and were transfected using transfection reagents with the helper plasmids, viral envelope and the DNA construct/plasmid of interest. After 48 h the lentiviral supernatant was harvested and filtered.

Infectious titre was determined by transducing SUP-T1 cell line with serial dilutions of vector. 3 days later, cells are harvested and stained for live/dead and CAR using anti-G4S-linked antibody (Cell Signaling Technology, Massachusetts, USA). CAR expression is determined using a flow cytometer and analysed to calculate an infectious titer (TU/mL) using the following equation: Titre (TU/mL)=[(% transduced cells/100)*Number of cells transduced]/sample volume*Dilution factor.

### Generation of Tregs

Regulatory T cells were purified and FACS sorted as CD4+CD25+CD127-CD45RA+ cells from healthy donors. Cells were activated using CTS Treg Xpander Beads (ThermoFisher Scientific) in X-Vivo 15 medium (Lonza) supplemented with 5% heat-inactivated Human AB serum (Grifols Bio Supplies Inc.) in the presence of Interleukin-2 (300 IU/ml). After 48 hours of activation, cells were transduced with lentiviral particles comprising the constructs shown in FIG. 14. Cells were further expanded and at day 14 cells were harvested and counted. Transduction efficiency was assessed at day 12 post transduction by detecting CAR expressing using anti-G4S linker antibody (Cell Signaling Technology, Massachusetts, USA), FOXP3 expression (Transcription Factor Staining Buffer Set, ThermoFisher Scientific), and expression of the constitutively active cytokine receptor using an antibody specific to the extracellular domain of the receptor.

Expansion fold was calculated by dividing the absolute number of viable cells at the end of expansion (day 14) by the absolute number of viable cells at the beginning of expansion (day 0).

### Treg Activation Assay

For analysis of CAR-dependent Treg activation, rested Tregs were cultured in the presence of 1) HEK cells expressing the target antigen for the CAR at a ratio of 5:1 Tregs: cell line; or 2) Peptide which is the target antigen for the CAR (2.5 ug/ml). Here, 0.1*106 Treg were cultured. As a negative control, Tregs were cultured in the absence of a stimulus or in the presence of wildtype HEK cells at a ratio of 5:1 Tregs: cell line. As a positive control, Tregs were cultured in the presence of anti-CD3/CD28 activation beads.

After 24 h cells were harvested and stained with anti-CD4, anti-G4S, anti-CACR, anti-CD69, anti-CD137 and anti-GARP antibodies. Cells were acquired on a flow cytometer and percentages of CD69, CD137 and GARP up-regulation after stimulation were calculated within the transduced cell populations.

### Treg Proliferation Assay

To assess the proliferative capacity of Tregs in response to antigen, rested Tregs labelled with CellTrace Violet dye (ThermoFisher Scientific) were cultured in the presence of: 1) B cells expressing the target antigen for the CAR at ratios of 5:1 (Tregs: cell line). As a negative control, Tregs were cultured in media alone or in the presence of wildtype B cells at a ratio of 5:1 (Tregs: cell line). As a positive control, Tregs were cultured in the presence of anti-CD3/CD28 activation beads. Cultures were supplemented with IL-2 (400 IU/ml). Six days after assay set-up, cells were stained with anti-CD4 and anti-G4S linker antibodies and acquired by flow cytometry. The fold change of proliferation for each condition was calculated by dividing the percentage of proliferation of that condition with its relevant control.

### Treg Suppression Assay

To assess the ability of Tregs to suppress effector T cell activation, Teff cells were labeled with CellTrace Violet dye (ThermoFisher Scientific). Labeled Teff cells were then co-cultured with different concentrations of rested Tregs (ratios Treg: Teff of 1:1, 1:2, 1:4, 1:8, 1:16, 1:32, 1:64). The cells were activated using either anti-CD3/CD28 beads, wildtype B cells or B cells expressing the target antigen for the CAR. Five days after assay set-up cells were harvested and analyzed by flow cytometry. CellTrace Violet dye dilution is used as a surrogate marker for Teff cell proliferation.

### Treg Survival Assay

To assess the ability of Tregs to survive in the absence of IL2, rested Tregs labelled with CellTrace Violet dye (ThermoFisher Scientific) were cultured in the presence of: 1) B cells expressing the target antigen for the CAR at ratios of 5:1 (Tregs: cell line). As a negative control, Tregs were cultured in media alone or in the presence of wildtype B cells at a ratio of 5:1 (Tregs: cell line). As a positive control, Tregs were cultured in the presence of anti-CD3/CD28 activation beads. Six days after assay set-up, cells were stained with anti-CD4 and anti-G4S linker antibodies and acquired by flow cytometry. The fold change of cell counts for each condition was calculated by dividing the percentage of proliferation of that condition with its relevant control.

### Results

Virus titres of each vector is shown in the table below.

| | Titre (TU/mL) |
|---|---|
| PGK-CAR-FOXP3 | 1.23 × 10⁹ |
| SFFV-CAR-FOXP3 | 3.72 × 10⁸ |
| PGK-CAR-FOXP3-CACR | 4.23 × 10⁸ |
| SFFV-CAR-FOXP3-CACR | 3.87 × 10⁸ |

Fold expansion and transduction (% and MFI) following the 14 day expansion protocol are shown in the table below.

| | PGK- | SFFV- | PGK-CAR- | SFFV-CAR- |
|---|---|---|---|---|
| | CAR- | CAR- | FOX- | FOX- |
| | FOXP3 | FOXP3 | CACR | CACR |
| Expansion fold | 1671 | 501 | 2049 | 1068 |
| CAR Expression | 35.9 | 35.7 | 20.5 | 18.2 |
| (%) | | | | |
| CAR Expression | 2441 | 4706 | 2930 | 4694 |
| (MFI) | | | | |
| FOXP3 | 99.9 | 99.9 | 99.9 | 99.9 |
| Expression (%) | | | | |
| FOXP3 | 28246 | 34106 | 31703 | 40362 |
| Expression (MFI) | | | | |
| CACR | N/A | N/A | 13 | 14.4 |
| Expression (%) | | | | |
| CACR | N/A | N/A | 1378 | 2100 |
| Expression (MFI) | | | | |

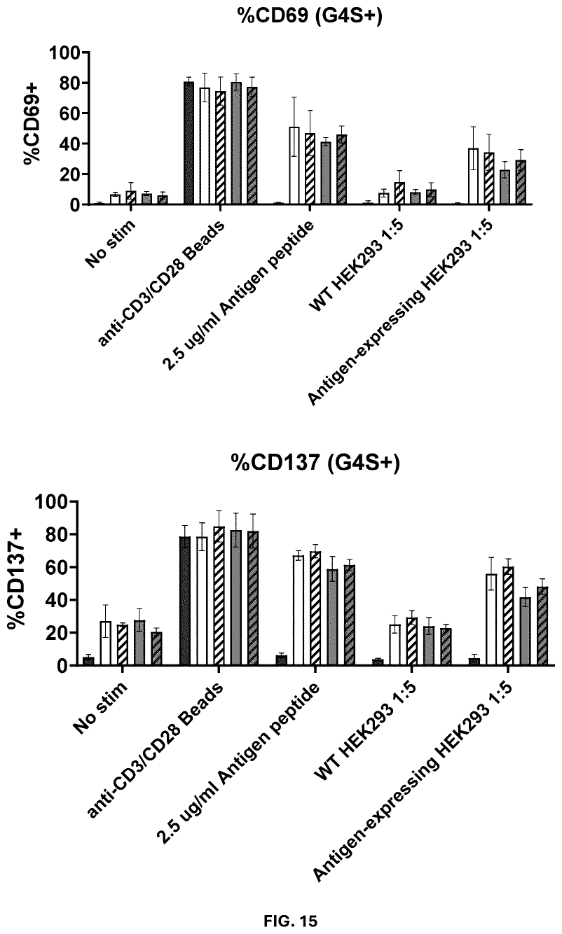

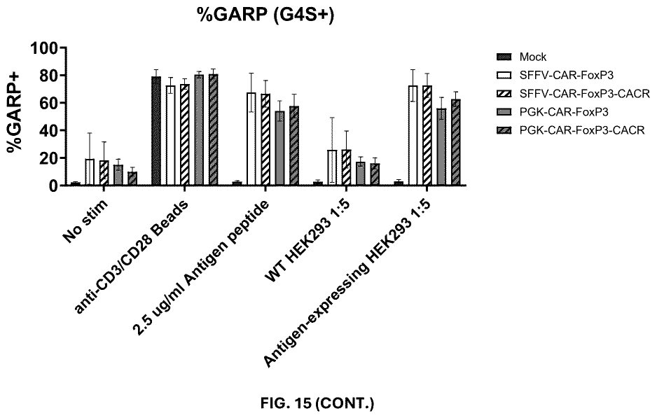

FIG. 15 shows the results of the activation assay, particularly % of cells expressing the activation markers CD69, CD137 and GARP (n=4). Antigen-specific activation is observed with all constructs.

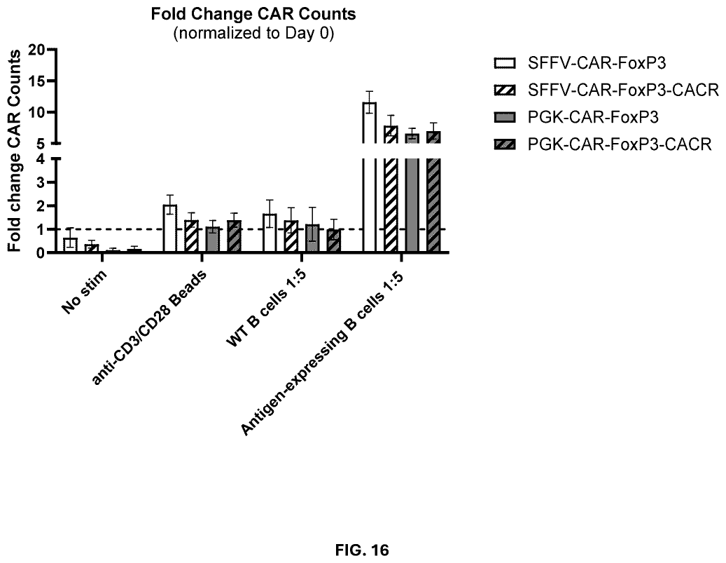

FIG. 16 shows the results of the proliferation assay (n=4). Antigen-specific CAR enrichment and proliferation is observed with all constructs with no major differences between promoters.

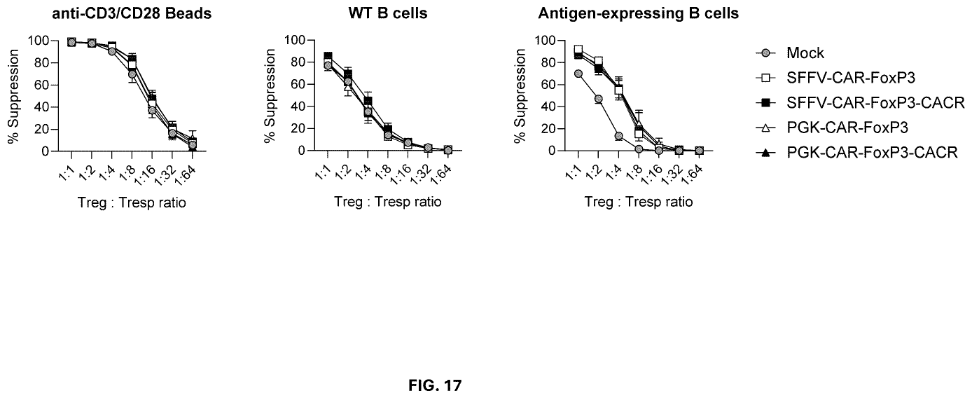

FIG. 17 shows the results of the suppression assay (n=3). Antigen-specific suppression is observed with all constructs with no major differences between promoters.

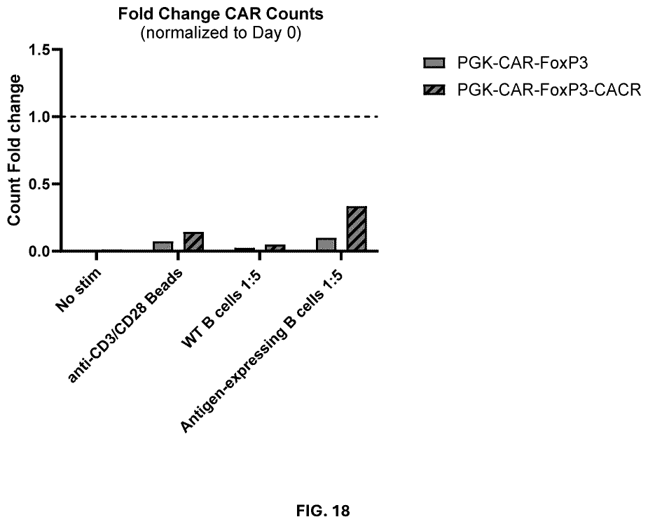

FIG. 18 shows the results of the survival assay (n=4). The presence of the constitutively active cytokine receptor (CACR) drives CAR enrichment in the absence of antigen or IL-2 compared to the constructs that do not include the CACR.

### Example 5

### Materials & Methods

Generation of Human Islet Microtissues (hIsMTs)

Human islet microtissues (hIsMTs) were prepared by InSphero AG (Zurich, Switzerland) using a scaffold-free, high-throughput reaggregation protocol. This method involves the dissociation of fresh or cryopreserved primary human islet cells, followed by controlled reassembly into uniform, standardized pseudoislets that recapitulate the native architecture and function of human pancreatic islets.

### Preparation of B Cell Lines

The Epstein-Barr virus-transformed B lymphoblastoid cell line (B-LCL) BM21 (ATCC) was cultured in RPMI 1640 supplemented with 10% fetal bovine serum (RPMI-10) at a density of 2×10⁵ cells/mL and passaged every 2-3 days. Prior to use in assays, the cells were irradiated at 120 Gray and added to the co-culture with hIsMTs at the specified cell numbers.

### Preparation of T Effector Cells (Naïve CD3 T Cells)

Naïve CD3+ T cells were isolated from freshly obtained peripheral blood mononuclear cells (PBMCs) using a commercial negative selection kit (StemCell Technologies), in accordance with the manufacturer's instructions. The isolated population (CD3⁺CD45RA⁺) exhibited a purity of 97.1%, as determined by flow cytometry following magnetic separation with the EasyEights™ Magnet (StemCell Technologies, purple variant). Isolated cells were cryopreserved and subsequently thawed prior to use in the co-culture assay.

### Preparation of T Regulatory Cells

Regulatory T (Treg) cells were fluorescence-activated cell sorted (FACS) as CD4⁺CD25⁺CD127⁻ cells from healthy donors. Cells were activated using Human T-Activator CD3/CD28 Dynabeads™ (Thermo Fisher Scientific) in X-Vivo medium (Lonza) supplemented with interleukin-2 (1000 IU/mL). After 48 hours of activation, cells were transduced with lentiviral particles encoding the ‘PGK-CAR-FOXP3’ construct shown in FIG. 1. Following transduction, cells were expanded for 15 days, cryopreserved and subsequently thawed prior to use in the co-culture assay.

Co-culture Assay with hIsMTs

For the co-culture assay with hIsMTs, Treg cells were resuspended in InSight™ Human Islet Maintenance Medium at 1×10⁶ cells/mL, effector T cells (Teff) at 2×10⁶ cells/mL, and B cells at 6×10⁵ cells/mL. Immune co-cultures were prepared in an intermediate 96-well plate by first adding 100 μL of InSight™ medium to wells in columns 2 through 5. Tregs were added at 200 μL per well to column 1, followed by a 2-fold serial dilution across columns 2 to 4 by transferring 100 μL between wells with gentle mixing and changing pipette tips between transfers. Column 5 served as the Teff-alone control with no Treg cells. Subsequently, 50 μL each of wild-type B cells and Teff cells were added to all wells in columns 1 through 5. The cell suspensions were mixed by gentle pipetting five times to ensure homogeneity. Finally, 50 μL of the resulting suspension was transferred to the assay plate containing the spheres. In parallel, control conditions were established by treating hIsMTs with recombinant cytokines-IL-1β (10 ng/ml), IFN-γ (50 ng/ml), and TNF-α (50 ng/mL), all from Sigma-Aldrich—prepared in phosphate-buffered saline (PBS) with 0.1% bovine serum albumin (BSA). Additional wells containing hIsMTs alone (i.e., without immune cells or cytokines) were included to assess basal responses. Co-cultures and controls were maintained for 3 days in a final volume of 100 UL at 37° C. in a humidified incubator with 5% CO₂.

3D Confocal Microscopy of hIsMTs

For NKX6.1 and INSULIN staining, islet microtissues were washed twice with PBS and fixed for 30 min in 4% paraformaldehyde (PFA) at room temperature. PFA was removed and islets were permeabilized with 0.5% Triton X-100 (in PBS) following blocking in PBS with 5% normal goat serum. MTs were incubated with rabbit anti-NKX6.1 (Abcam) and rat anti-INS overnight in 5% normal goat serum followed by goat anti-Rabbit Alexa Fluor 568 (Thermo Fisher), goat anti-Rat Alexa Fluor 488 (Thermo Fisher) and DAPI (Sigma) (1 ug/mL) staining for 4 hour in antibody incubation buffer (10% FCS, 0.2% Triton X-100 in PBS). Unspecific binding was removed with repeated wash steps in 0.2% Triton X-100 in PBS after both antibody incubations. Stained MTs were then transferred to Akura™384 Spheroid Microplates (InSphero), cleared with ScaleS4 (40 w/v % D-(−) sorbitol, 10 w/v % glycerol, 4 M Urea, 0.2% w/V % Triton X-100, and 15 v/v % DMSO in MilliQ water) and imaged with Yokogawa CQ1 confocal benchtop high-content analysis (HCA) system (Yokogawa Electric Corp) using a 40× dry objective (Olympus, Tokyo, Japan).

### Results

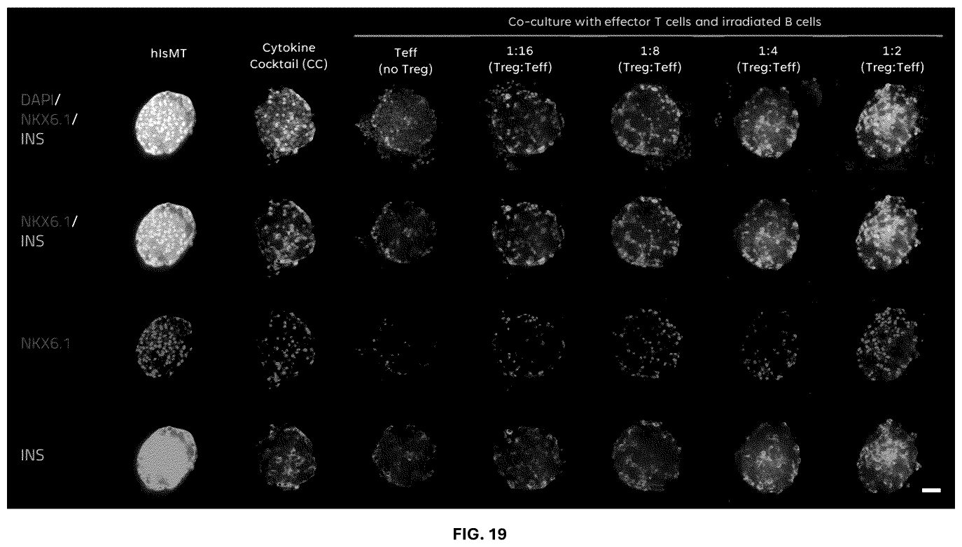

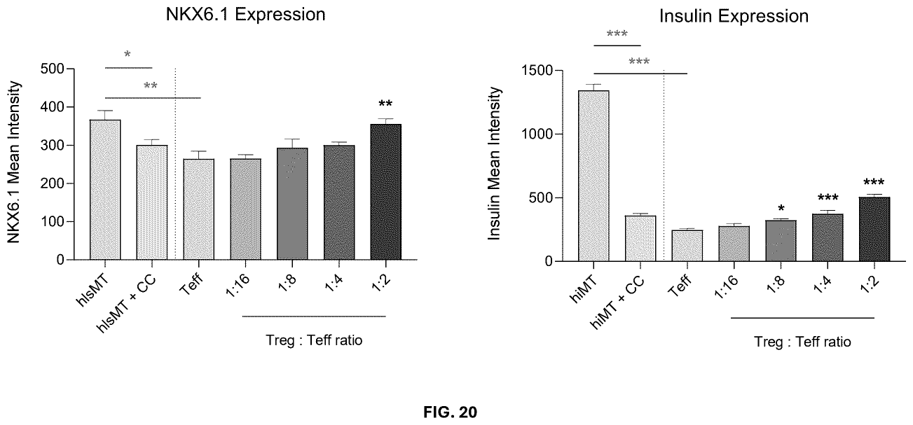

The results are shown in FIGS. 19 and 20.

Human islet MTs cultured alone exhibited strong expression of NKX6.1, a transcription factor essential for the maintenance of pancreatic β cell identity, as well as high levels of INS (insulin) expression. Treatment with a pro-inflammatory cytokine cocktail (CC) or co-culture with activated effector T cells (Teff only) —stimulated via irradiated B cells—led to a pronounced reduction in both NKX6.1 and INS expression, indicating substantial β cell injury (positive controls). Notably, the inclusion of Treg cells in these immune co-cultures which are also activated by the irradiated B cell population, substantially attenuated T cell-mediated cytotoxicity. Treg preserved NKX6.1 expression and maintained higher insulin levels compared to cultures exposed to Teff cells. This protective effect was dose-dependent, with increasing Teff-to-Treg ratios corresponding to greater preservation of β cell phenotype.

1. An expression construct comprising a PGK promoter operably linked to:
(i) a first nucleotide sequence encoding a chimeric antigen receptor (CAR); and
(ii) a second nucleotide sequence encoding a FOXP3 polypeptide;
wherein the first nucleotide sequence is located upstream of the second nucleotide sequence.

2. The expression construct of claim 1, wherein the expression construct does not comprise any other coding nucleotide sequence.

3. The expression construct of claim 1, wherein the PGK promoter comprises or consists of the sequence of SEQ ID NO: 195 or a functional variant or functional fragment thereof, optionally wherein the PGK promoter is a truncated human PGK promoter or a functional variant having at least 70% identity thereto, optionally wherein the truncated human PGK promoter has the sequence of SEQ ID NO: 196, SEQ ID NO: 197, SEQ ID NO: 198 or SEQ ID NO: 199.

4. The expression construct of claim 1, wherein the CAR is a human CAR and/or wherein the CAR comprises an exodomain comprising an antigen recognition domain.

5. The expression construct of claim 4, wherein the antigen recognition domain specifically binds to ENTPD3.

6. The expression construct of claim 4, wherein the antigen recognition domain is an antibody, an antibody fragment, or derived from an antibody, optionally wherein the antigen recognition domain is a single chain antibody (scFv).

7. The expression construct of claim 4, wherein the antigen recognition domain comprises:
i. VH CDR1, 2 and 3 sequences set forth in SEQ ID NOs: 7, 8 and 9 respectively and VL CDR1, 2 and 3 sequences set forth in SEQ ID NOs: 10, 11 and 12 respectively;
ii. VH CDR1, 2 and 3 sequences set forth in SEQ ID NOs: 19, 20 and 21 respectively and VL CDR1, 2 and 3 sequences set forth in SEQ ID NOs: 22, 23 and 24 respectively;
iii. VH CDR1, 2 and 3 sequences set forth in SEQ ID NOs: 73, 74 and 75 respectively and VL CDR1, 2 and 3 sequences set forth in SEQ ID NOs: 76, 77 and 78 respectively;
iv. VH CDR1, 2 and 3 sequences set forth in SEQ ID NOs: 25, 26 and 27 respectively and VL CDR1, 2 and 3 sequences set forth in SEQ ID NOs: 28, 29 and 30 respectively;
v. VH CDR1, 2 and 3 sequences set forth in SEQ ID NOs: 37, 38 and 39 respectively and VL CDR1, 2 and 3 sequences set forth in SEQ ID NOs: 40, 41 and 42 respectively;
vi. VH CDR1, 2 and 3 sequences set forth in SEQ ID NOs: 1, 2 and 3 respectively and VL CDR1, 2 and 3 sequences set forth in SEQ ID NOs: 4, 5 and 6 respectively;
vii. VH CDR1, 2 and 3 sequences set forth in SEQ ID NOs: 13, 14 and 15 respectively and VL CDR1, 2 and 3 sequences set forth in SEQ ID NOs: 16, 17 and 18 respectively;
viii. VH CDR1, 2 and 3 sequences set forth in SEQ ID NOs: 31, 32 and 33 respectively and VL CDR1, 2 and 3 sequences set forth in SEQ ID NOs: 34, 35 and 36 respectively;
ix. VH CDR1, 2 and 3 sequences set forth in SEQ ID NOs: 43, 44 and 45 respectively and VL CDR1, 2 and 3 sequences set forth in SEQ ID NOs: 46, 47 and 48 respectively;
x. VH CDR1, 2 and 3 sequences set forth in SEQ ID NOs: 49, 50 and 51 respectively and VL CDR1, 2 and 3 sequences set forth in SEQ ID NOs: 52, 53 and 54 respectively;
xi. VH CDR1, 2 and 3 sequences set forth in SEQ ID NOs: 55, 56 and 57 respectively and VL CDR1, 2 and 3 sequences set forth in SEQ ID NOs: 58, 59 and 60 respectively;
xii. VH CDR1, 2 and 3 sequences set forth in SEQ ID NOs: 61, 62 and 63 respectively and VL CDR1, 2 and 3 sequences set forth in SEQ ID NOs: 64, 65 and 66 respectively;
xiii. VH CDR1, 2 and 3 sequences set forth in SEQ ID NOs: 67, 68 and 69 respectively and VL CDR1, 2 and 3 sequences set forth in SEQ ID NOs: 70, 71 and 72 respectively; or
xiv. VH CDR1, 2 and 3 sequences set forth in SEQ ID NOs: 79, 80 and 81 respectively and VL CDR1, 2 and 3 sequences set forth in SEQ ID NOs: 82, 83 and 84 respectively;
wherein one or more of said CDR sequences of (i) to (xiv) may optionally comprise 1 to 3 amino acid modifications relative to an aforementioned CDR sequence, particularly wherein one or more of said CDR sequences may optionally be modified by substitution, addition or deletion of 1 to 3 amino acids.

8. The expression construct of claim 4, wherein the antigen recognition domain comprises:
i. a VH domain comprising the sequence set forth in SEQ ID NO: 87, or a sequence having at least 70% sequence identity thereto, and a VL domain comprising the sequence as set forth in SEQ ID NO: 88, or a sequence having at least 70% identity thereto;
ii. a VH domain comprising the sequence set forth in SEQ ID NO: 91, or a sequence having at least 70% sequence identity thereto, and a VL domain comprising the sequence as set forth in SEQ ID NO: 92, or a sequence having at least 70% identity thereto;
iii. a VH domain comprising the sequence set forth in SEQ ID NO: 109, or a sequence having at least 70% sequence identity thereto, and a VL domain comprising the sequence as set forth in SEQ ID NO: 110, or a sequence having at least 70% identity thereto;
iv. a VH domain comprising the sequence set forth in SEQ ID NO: 93, or a sequence having at least 70% sequence identity thereto, and a VL domain comprising the sequence as set forth in SEQ ID NO: 94, or a sequence having at least 70% identity thereto;
v. a VH domain comprising the sequence set forth in SEQ ID NO: 97, or a sequence having at least 70% sequence identity thereto, and a VL domain comprising the sequence as set forth in SEQ ID NO: 98, or a sequence having at least 70% identity thereto;
vi. a VH domain comprising the sequence set forth in SEQ ID NO: 85, or a sequence having at least 70% sequence identity thereto, and a VL domain comprising the sequence as set forth in SEQ ID NO: 86, or a sequence having at least 70% identity thereto;
vii. a VH domain comprising the sequence set forth in SEQ ID NO: 89, or a sequence having at least 70% sequence identity thereto, and a VL domain comprising the sequence as set forth in SEQ ID NO: 90, or a sequence having at least 70% identity thereto;
viii. a VH domain comprising the sequence set forth in SEQ ID NO: 95, or a sequence having at least 70% sequence identity thereto, and a VL domain comprising the sequence as set forth in SEQ ID NO: 96, or a sequence having at least 70% identity thereto;
ix. a VH domain comprising the sequence set forth in SEQ ID NO: 99, or a sequence having at least 70% sequence identity thereto, and a VL domain comprising the sequence as set forth in SEQ ID NO: 100, or a sequence having at least 70% identity thereto;
x. a VH domain comprising the sequence set forth in SEQ ID NO: 101, or a sequence having at least 70% sequence identity thereto, and a VL domain comprising the sequence as set forth in SEQ ID NO: 102, or a sequence having at least 70% identity thereto;
xi. a VH domain comprising the sequence set forth in SEQ ID NO: 103, or a sequence having at least 70% sequence identity thereto, and a VL domain comprising the sequence as set forth in SEQ ID NO: 104, or a sequence having at least 70% identity thereto;
xii. a VH domain comprising the sequence set forth in SEQ ID NO: 105, or a sequence having at least 70% sequence identity thereto, and a VL domain comprising the sequence as set forth in SEQ ID NO: 106, or a sequence having at least 70% identity thereto;
xiii. a VH domain comprising the sequence set forth in SEQ ID NO: 107, or a sequence having at least 70% sequence identity thereto, and a VL domain comprising the sequence as set forth in SEQ ID NO: 108, or a sequence having at least 70% identity thereto; or
xiv. a VH domain comprising the sequence set forth in SEQ ID NO: 111, or a sequence having at least 70% sequence identity thereto, and a VL domain comprising the sequence as set forth in SEQ ID NO: 112, or a sequence having at least 70% identity thereto.

9. The expression construct of claim 4, wherein the antigen recognition domain comprises or consists of:
i. the sequence set forth in SEQ ID NO: 114 or a sequence having at least 70% sequence identity thereto;
ii. the sequence set forth in SEQ ID NO: 116 or a sequence having at least 70% sequence identity thereto;
iii. the sequence set forth in SEQ ID NO: 125 or a sequence having at least 70% sequence identity thereto;
iv. the sequence set forth in SEQ ID NO: 117 or a sequence having at least 70% sequence identity thereto;
v. the sequence set forth in SEQ ID NO: 119 or a sequence having at least 70% sequence identity thereto;
vi. the sequence set forth in SEQ ID NO: 113 or a sequence having at least 70% sequence identity thereto;
vii. the sequence set forth in SEQ ID NO: 115 or a sequence having at least 70% sequence identity thereto;
viii. the sequence set forth in SEQ ID NO: 118 or a sequence having at least 70% sequence identity thereto;
ix. the sequence set forth in SEQ ID NO: 120 or a sequence having at least 70% sequence identity thereto;
x. the sequence set forth in SEQ ID NO: 121 or a sequence having at least 70% sequence identity thereto;
xi. the sequence set forth in SEQ ID NO: 122 or a sequence having at least 70% sequence identity thereto;
xii. the sequence set forth in SEQ ID NO: 123 or a sequence having at least 70% sequence identity thereto;
xiii. the sequence set forth in SEQ ID NO: 124 or a sequence having at least 70% sequence identity thereto; or
xiv. the sequence set forth in SEQ ID NO: 126 or a sequence having at least 70% sequence identity thereto.

10. The expression construct of claim 1, wherein the CAR comprises or consists of the sequence of any one of SEQ ID NOs: 127 to 152 or a functional variant thereof having at least 70% sequence identity thereto, particularly wherein the CAR comprises or consists of the sequence of SEQ ID NO: 128 or 130 or a functional variant having at least 70% sequence identity thereto.

11. The expression construct of claim 1, wherein the FOXP3 polypeptide has the sequence of SEQ ID NO: 200, SEQ ID NO: 201, SEQ ID NO: 202, SEQ ID NO: 203, SEQ ID NO: 204, SEQ ID NO: 205 or a variant having at least 70% identity thereto, or the second nucleotide sequence encoding the FOXP3 polypeptide has the sequence of SEQ ID NO: 206, SEQ ID NO: 207 or a sequence having at least 80% identity thereto.

12. The expression construct of claim 1, wherein the expression construct comprises the sequence of SEQ ID NO: 214 or a sequence having at least 70% identity thereto.

13. A nucleic acid molecule comprising an expression construct of claim 1, optionally wherein the nucleic acid molecule does not comprise any other coding nucleotide sequence.

14. A vector comprising the expression construct of claim 1 or a nucleic acid molecule comprising said expression construct, optionally wherein the vector is a plasmid or a viral vector, optionally a lentiviral vector or gamma-retroviral vector.

15. A cell comprising the expression construct of claim 1 or a nucleic acid molecule comprising said expression construct, or a vector comprising said expression construct or nucleic acid molecule.

16. The cell of claim 15, wherein the cell is a production host cell or an immune cell or a progenitor or precursor thereof, for example a T cell or precursor thereof, or a stem cell, optionally wherein the cell is a regulatory T cell (Treg) or a precursor thereof, or an iPSC cell.

17. A pharmaceutical composition comprising the cell of claim 15 or a cell population, wherein the cell population comprises the cell of claim 15 or a plurality of cells of claim 15, optionally wherein said plurality of cells is a plurality of T cells or a plurality of Tregs.

18. A method of adoptive cell transfer therapy, said method comprising administering to a subject the cell of claim 15, optionally wherein the cell is a Treg.

19. A method of inducing tolerance to a transplant, treating and/or preventing graft-versus-host disease (GvHD), treating or preventing an infectious, allergic, autoimmune or inflammatory disease, inducing immunosuppression, or for use in promoting tissue repair and/or tissue regeneration, optionally wherein the autoimmune or inflammatory disease is type 1 diabetes, said method comprising administering to a subject a cell of claim 15, optionally wherein the cell is a Treg.

20. A method of making the cell of claim 15, which comprises the step of introducing into the cell an expression construct comprising a PGK promoter operably linked to: (i) a first nucleotide sequence encoding a chimeric antigen receptor (CAR) and (ii) a second nucleotide sequence encoding a FOXP3 polypeptide, wherein the first nucleotide sequence is located upstream of the second nucleotide sequence.

21. A pharmaceutical composition comprising the vector of claim 14.

22. A method of inducing tolerance to a transplant, treating and/or preventing graft-versus-host disease (GvHD), treating or preventing an infectious, allergic, autoimmune or inflammatory disease, or inducing immunosuppression, or promoting tissue repair and/or tissue regeneration, optionally wherein the autoimmune or inflammatory disease is type 1 diabetes, said method comprising administering to a subject the pharmaceutical composition of claim 21.


SEQUENCE LISTING (274 sequences)

SEQ ID NO: 1 - protein, 8 aa, synthetic construct
GFTFDDYA

SEQ ID NO: 2 - protein, 8 aa, synthetic construct
ISWNSGSI

SEQ ID NO: 3 - protein, 20 aa, synthetic construct
AKGANYDILTGYRKDNWFDP

SEQ ID NO: 4 - protein, 8 aa, synthetic construct
SSNIGSNY

SEQ ID NO: 5 - skipped

SEQ ID NO: 6 - protein, 11 aa, synthetic construct
AAWDDSLSGWV

SEQ ID NO: 7 - protein, 8 aa, synthetic construct
GFTFSSYA

SEQ ID NO: 8 - protein, 8 aa, synthetic construct
ISGSGGNT

SEQ ID NO: 9 - protein, 14 aa, synthetic construct
AKGGSTSSGYRFDY

SEQ ID NO: 10 - protein, 7 aa, synthetic construct
SGHSNYA

SEQ ID NO: 11 - protein, 7 aa, synthetic construct
VNRDGSY

SEQ ID NO: 12 - protein, 9 aa, synthetic construct
QTWGTGVQV

SEQ ID NO: 13 - protein, 8 aa, synthetic construct
GYTFTSYA

SEQ ID NO: 14 - protein, 8 aa, synthetic construct
INAGNGNT

SEQ ID NO: 15 - protein, 11 aa, synthetic construct
ARDSGYDLFDY

SEQ ID NO: 16 - protein, 9 aa, synthetic construct
SGSVSTTYY

SEQ ID NO: 17 - skipped

SEQ ID NO: 18 - protein, 10 aa, synthetic construct
LLYMGSGVWV

SEQ ID NO: 19 - protein, 8 aa, synthetic construct
GYSFTSYW

SEQ ID NO: 20 - protein, 8 aa, synthetic construct
IYPGDSGT

SEQ ID NO: 21 - protein, 11 aa, synthetic construct
ARQQGAGAFDI

SEQ ID NO: 22 - protein, 9 aa, synthetic construct
SRDVGGYNY

SEQ ID NO: 23 - skipped

SEQ ID NO: 24 - protein, 10 aa, synthetic construct
SSYTSSSTVV

SEQ ID NO: 25 - protein, 8 aa, synthetic construct
GFTFGDYA

SEQ ID NO: 26 - protein, 10 aa, synthetic construct
IRSKAYGGTT

SEQ ID NO: 27 - protein, 15 aa, synthetic construct
TRVPVVAGWYNWFDP

SEQ ID NO: 28 - protein, 8 aa, synthetic construct
SSNIGSNY

SEQ ID NO: 29 - skipped

SEQ ID NO: 30 - protein, 10 aa, synthetic construct
SSYAGSNNVV

SEQ ID NO: 31 - protein, 8 aa, synthetic construct
GFTFDDYA

SEQ ID NO: 32 - protein, 8 aa, synthetic construct
ISWNSGSI

SEQ ID NO: 33 - protein, 20 aa, synthetic construct
AKGANYDILTGYRKDNWFDP

SEQ ID NO: 34 - protein, 6 aa, synthetic construct
QTISNW

SEQ ID NO: 35 - skipped

SEQ ID NO: 36 - protein, 9 aa, synthetic construct
QQYHSYSRT

SEQ ID NO: 37 - protein, 8 aa, synthetic construct
GFTFDDYA

SEQ ID NO: 38 - protein, 8 aa, synthetic construct
ISWNSGSI

SEQ ID NO: 39 - protein, 14 aa, synthetic construct
AKGANYDILTGYEY

SEQ ID NO: 40 - protein, 9 aa, synthetic construct
TGAVTSDHH

SEQ ID NO: 41 - skipped

SEQ ID NO: 42 - protein, 9 aa, synthetic construct
FLYYSGTAI

SEQ ID NO: 43 - protein, 8 aa, synthetic construct
GFTFSSYA

SEQ ID NO: 44 - protein, 8 aa, synthetic construct
ISGSGGST

SEQ ID NO: 45 - protein, 15 aa, synthetic construct
AKDDYDFWSGSLGNY

SEQ ID NO: 46 - protein, 8 aa, synthetic construct
SSNIGSNY

SEQ ID NO: 47 - skipped

SEQ ID NO: 48 - protein, 11 aa, synthetic construct
AAWDDTLNAWV

SEQ ID NO: 49 - protein, 8 aa, synthetic construct
GFTFDDYA

SEQ ID NO: 50 - protein, 8 aa, synthetic construct
ISWNSGSI

SEQ ID NO: 51 - protein, 20 aa, synthetic construct
AKGANYDILTGYRKDNWFDP

SEQ ID NO: 52 - protein, 6 aa, synthetic construct
QSVGSS

SEQ ID NO: 53 - skipped

SEQ ID NO: 54 - protein, 10 aa, synthetic construct
QQRSNWPPYT

SEQ ID NO: 55 - protein, 8 aa, synthetic construct
GFTFSSYG

SEQ ID NO: 56 - protein, 8 aa, synthetic construct
ISYDGSNK

SEQ ID NO: 57 - protein, 15 aa, synthetic construct
AKDHHPYGSSDSFDY

SEQ ID NO: 58 - protein, 9 aa, synthetic construct
TGAVTRGHY

SEQ ID NO: 59 - skipped

SEQ ID NO: 60 - protein, 14 aa, synthetic construct
LLSFIDTRYPARYV

SEQ ID NO: 61 - protein, 8 aa, synthetic construct
GYTFTSYG

SEQ ID NO: 62 - protein, 8 aa, synthetic construct
ISAYNGNT

SEQ ID NO: 63 - protein, 16 aa, synthetic construct
ARDDPWGSYRPRPFDY

SEQ ID NO: 64 - protein, 8 aa, synthetic construct
SSNIGSNT

SEQ ID NO: 65 - skipped

SEQ ID NO: 66 - protein, 11 aa, synthetic construct
AAWDDSLNGWV

SEQ ID NO: 67 - protein, 8 aa, synthetic construct
GGTFSSYA

SEQ ID NO: 68 - protein, 8 aa, synthetic construct
IIPIFGTA

SEQ ID NO: 69 - protein, 13 aa, synthetic construct
ARGASGYDWSLDY

SEQ ID NO: 70 - protein, 11 aa, synthetic construct
QSLVYSDGNTY

SEQ ID NO: 71 - skipped

SEQ ID NO: 72 - protein, 9 aa, synthetic construct
MQGSRWPPT

SEQ ID NO: 73 - protein, 8 aa, synthetic construct
GYSFISHW

SEQ ID NO: 74 - protein, 8 aa, synthetic construct
IYPGDSGT

SEQ ID NO: 75 - protein, 12 aa, synthetic construct
ARLADGGLQFDH

SEQ ID NO: 76 - protein, 9 aa, synthetic construct
SSDVGGYNY

SEQ ID NO: 77 - skipped

SEQ ID NO: 78 - protein, 10 aa, synthetic construct
NSYTSSSTYV

SEQ ID NO: 79 - protein, 8 aa, synthetic construct
GFTFSSYW

SEQ ID NO: 80 - protein, 8 aa, synthetic construct
IKQDGSEK

SEQ ID NO: 81 - protein, 13 aa, synthetic construct
ARVPNYYDSSGTV

SEQ ID NO: 82 - protein, 8 aa, synthetic construct
SGSIASNY

SEQ ID NO: 83 - skipped

SEQ ID NO: 84 - protein, 9 aa, synthetic construct
QSYDSTLLV

SEQ ID NO: 85 - protein, 127 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSS

SEQ ID NO: 86 - protein, 110 aa, synthetic construct
LPVLTQPPSASGTPGQKVTISCSGSSSNIGSNYVFWYEQLPGAAPKLLMYSSNQRPSGVPDRFSGSKSGTSASLAISGLRSEDEADYYCAAWDDSLSGWVFGGGTKLTVL

SEQ ID NO: 87 - protein, 121 aa, synthetic construct
QVQLVQSGGGVVQPGRSLRLSCAASGFTFSSYAMSWVRQAPGKGLEWVSAISGSGGNTNYADSVKGRFTISRDNSKNTLYLQMNSLRAEDTAVYYCAKGGSTSSGYRFDYWGQGTLVTVSS

SEQ ID NO: 88 - protein, 111 aa, synthetic construct
QLVLTQSPSASASLGASVKLTCTLSSGHSNYAIAWHQQQPEKGPRFLMNVNRDGSYTKGDGIPDRFSGSSSGAERYLTISSLQSDDEADYYCQTWGTGVQVFGGGTKLTVL

SEQ ID NO: 89 - protein, 118 aa, synthetic construct
QVQLQQSGAEVKKPGASVKVSCKASGYTFTSYAMHWVRQAPGQRLEWMGWINAGNGNTKYSQKLQGRVTITRDTSASTAYMELSSLRSEDTAVYYCARDSGYDLFDYWGQGTLVTVSS

SEQ ID NO: 90 - protein, 110 aa, synthetic construct
QAVVTQEPSFSVSPGGTVTLTCGLTSGSVSTTYYPSWYQQTPGQTPRTLIYKTNLRSPGVPDRFSGSILGNKAALTITGAQADDDSDYYCLLYMGSGVWVFGGGTRLTVL

SEQ ID NO: 91 - protein, 118 aa, synthetic construct
QVQLVQSGAEVKKPGESLKISCKGSGYSFTSYWIGWVRQMPGKGLEWMGIIYPGDSGTRYSPSFQGQVTISADKSISTAYLQWSSLKASNTAMYYCARQQGAGAFDIWGQGTMVTVSS

SEQ ID NO: 92 - protein, 110 aa, synthetic construct
QSALTQPPSASGSPGQSVTISCTGTSRDVGGYNYVSWYQQHPGKAPKLILYEVTKRPSGVPDRFSGSKSGNTASLTISGLQAEDEADYYCSSYTSSSTVVFGGGTKLTVL

SEQ ID NO: 93 - protein, 124 aa, synthetic construct
EVQLVQSGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPVVAGWYNWFDPWGQGTLVTVSS

SEQ ID NO: 94 - protein, 109 aa, synthetic construct
QSVLTQPPSASGTPGQRVTISCSGSSSNIGSNYVYWYQQLPGTAPKLLIYRNNQRPSGVPDRFSGSKSGNTASLTVSGLQAGDEADYYCSSYAGSNNVVFGGGTKLAVL

SEQ ID NO: 95 - protein, 127 aa, synthetic construct; identical to SEQ ID NO: 85

SEQ ID NO: 96 - protein, 106 aa, synthetic construct
DIVMTQTPSTLSASVGDRVTITCRASQTISNWLAWYQQKPGKAPKLLIYKASTLESGVPSRFSGTGSGTEFTLTISSLQPDDFATYFCQQYHSYSRTFGQGTKVDV

SEQ ID NO: 97 - protein, 121 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYEYWGQGTLVTVSS

SEQ ID NO: 98 - protein, 109 aa, synthetic construct
QAVVTQEPSLTVSPGETVTLTCGSSTGAVTSDHHPYWFQQKPGQVPRTLMHDTSTRYSWTPARFSGSIVGGKAALTLSGAQPEDEAEYHCFLYYSGTAIFGGGTKLTVL

SEQ ID NO: 99 - protein, 122 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDYDFWSGSLGNYWGQGTLVTVSS

SEQ ID NO: 100 - protein, 110 aa, synthetic construct
QSVLTQPPSASGTPGQRVTISCSGRSSNIGSNYVYWYQQPPGTAPKLLIYENNHRPSGVPDRFSASKSGTSASLAISGLRSEDEADYYCAAWDDTLNAWVFGGGTKLTVL

SEQ ID NO: 101 - protein, 127 aa, synthetic construct; identical to SEQ ID NO: 85

SEQ ID NO: 102 - protein, 108 aa, synthetic construct
DIVMTQSPATLSLSPGERATLSCRASQSVGSSLAWFQQKPGQAPRLLIYDASNRATGIPARFSGSGSGTDFTLTISSLEPEDFAVYYCQQRSNWPPYTFGQGTKLEIK

SEQ ID NO: 103 - protein, 122 aa, synthetic construct
QMQLVQSGGGVVQPGRSLRLSCAASGFTFSSYGMHWVRQAPGKGLERVAVISYDGSNKYYADSVKGRFTISRDNSKNTLYLQMNSLRAEDTALYYCAKDHHPYGSSDSFDYWGQGTLVTVSS

SEQ ID NO: 104 - protein, 114 aa, synthetic construct
QAVVTQEPSLTVSPGGTVTLTCGSSTGAVTRGHYPYWLQQKPGQAPRTLIYDTVKKHSWTPARFSGSLVGGKAALTLSGAQPEDEADYYCLLSFIDTRYPARYVFGTGTKVTVL

SEQ ID NO: 105 - protein, 123 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDDPWGSYRPRPFDYWGQGTLVTVSS

SEQ ID NO: 106 - protein, 110 aa, synthetic construct
QSVLTQPPSASGTPGQRVTISCSGSSSNIGSNTVNWYQQLPGTTPKLLIYSNNQRPSGVPDRFSGSKSGTSASLAISGLQSEDEADYYCAAWDDSLNGWVFGGGTKLTVL

SEQ ID NO: 107 - protein, 120 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGASGYDWSLDYWGQGTLVTVSS

SEQ ID NO: 108 - protein, 112 aa, synthetic construct
KIVMTQSPLSLPVTLGQPASISCRSSQSLVYSDGNTYLNWFQQRPGQSPRRLIYKVSNRHSGVPDRFSGSGSGTDFTLKISRVEAEDVGVYFCMQGSRWPPTFGPGTKVEIK

SEQ ID NO: 109 - protein, 119 aa, synthetic construct
QIQLVQSGTEVKKPGESLKISCKGSGYSFISHWIGWVRQMPGKGLEWMGIIYPGDSGTRYSPSFQGQATISADKSISTAYLQWSSLKASDTAMYYCARLADGGLQFDHWGQGTLVTVSS

SEQ ID NO: 110 - protein, 110 aa, synthetic construct
QSALTQPASVSGSPGQSITISCTGTSSDVGGYNYVSWYQQHPGKAPKLMIYGVSNRPSGVSNRFSGSKSGNTASLTISGLQAEDEADYYCNSYTSSSTYVFGTGTKVTVL

SEQ ID NO: 111 - protein, 120 aa, synthetic construct
EVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARVPNYYDSSGTVWGQGTLVTVSS

SEQ ID NO: 112 - protein, 110 aa, synthetic construct
NFMLTQPHSVSESPGKTVTISCTRSSGSIASNYVQWYQQRPGSAPTTVIYEDNQRPSGVPDRFSGSIDSSSNSASLTISGLEPEDEADYYCQSYDSTLLVFGGGTKLTVL

SEQ ID NO: 113 - protein, 252 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSLPVLTQPPSASGTPGQKVTISCSGSSSNIGSNYVFWYEQLPGAAPKLLMYSSNQRPSGVPDRFSGSKSGTSASLAISGLRSEDEADYYCAAWDDSLSGWVFGGGTKLTVL

SEQ ID NO: 114 - protein, 247 aa, synthetic construct
QVQLVQSGGGVVQPGRSLRLSCAASGFTFSSYAMSWVRQAPGKGLEWVSAISGSGGNTNYADSVKGRFTISRDNSKNTLYLQMNSLRAEDTAVYYCAKGGSTSSGYRFDYWGQGTLVTVSSGGGGSGGGGSGGGGSQLVLTQSPSASASLGASVKLTCTLSSGHSNYAIAWHQQQPEKGPRFLMNVNRDGSYTKGDGIPDRFSGSSSGAERYLTISSLQSDDEADYYCQTWGTGVQVFGGGTKLTVL

SEQ ID NO: 115 - protein, 243 aa, synthetic construct
QVQLQQSGAEVKKPGASVKVSCKASGYTFTSYAMHWVRQAPGQRLEWMGWINAGNGNTKYSQKLQGRVTITRDTSASTAYMELSSLRSEDTAVYYCARDSGYDLFDYWGQGTLVTVSSGGGGSGGGGSGGGGSQAVVTQEPSFSVSPGGTVTLTCGLTSGSVSTTYYPSWYQQTPGQTPRTLIYKTNLRSPGVPDRFSGSILGNKAALTITGAQADDDSDYYCLLYMGSGVWVFGGGTRLTVL

SEQ ID NO: 116 - protein, 243 aa, synthetic construct
QVQLVQSGAEVKKPGESLKISCKGSGYSFTSYWIGWVRQMPGKGLEWMGIIYPGDSGTRYSPSFQGQVTISADKSISTAYLQWSSLKASNTAMYYCARQQGAGAFDIWGQGTMVTVSSGGGGSGGGGSGGGGSQSALTQPPSASGSPGQSVTISCTGTSRDVGGYNYVSWYQQHPGKAPKLILYEVTKRPSGVPDRFSGSKSGNTASLTISGLQAEDEADYYCSSYTSSSTVVFGGGTKLTVL

SEQ ID NO: 117 - protein, 248 aa, synthetic construct
EVQLVQSGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPVVAGWYNWFDPWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGNTASLTVSGLQAGDEADYYCSSYAGSNNVVFGGGTKLAVL

SEQ ID NO: 118 - protein, 248 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQTPSTLSASVGDRVTITCRASQTISNWLAWYQQKPGKAPKLLIYKASTLESGVPSRFSGTGSGTEFTLTISSLQPDDFATYFCQQYHSYSRTFGQGTKVDV

SEQ ID NO: 119 - protein, 245 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYEYWGQGTLVTVSSGGGGSGGGGSGGGGSQAVVTQEPSLTVSPGETVTLTCGSSTGAVTSDHHPYWFQQKPGQVPRTLMHDTSTRYSWTPARFSGSIVGGKAALTLSGAQPEDEAEYHCFLYYSGTAIFGGGTKLTVL

SEQ ID NO: 120 - protein, 247 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDYDFWSGSLGNYWGQGTLVTVSSGGGGSGGGGSGGGGSQSVLTQPPSASGTPGQRVTISCSGRSSNIGSNYVYWYQQPPGTAPKLLIYENNHRPSGVPDRFSASKSGTSASLAISGLRSEDEADYYCAAWDDTLNAWVFGGGTKLTVL

SEQ ID NO: 121 - protein, 250 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQSPATLSLSPGERATLSCRASQSVGSSLAWFQQKPGQAPRLLIYDASNRATGIPARFSGSGSGTDFTLTISSLEPEDFAVYYCQQRSNWPPYTFGQGTKLEIK

SEQ ID NO: 122 - protein, 251 aa, synthetic construct
QMQLVQSGGGVVQPGRSLRLSCAASGFTFSSYGMHWVRQAPGKGLERVAVISYDGSNKYYADSVKGRFTISRDNSKNTLYLQMNSLRAEDTALYYCAKDHHPYGSSDSFDYWGQGTLVTVSSGGGGSGGGGSGGGGSQAVVTQEPSLTVSPGGTVTLTCGSSTGAVTRGHYPYWLQQKPGQAPRTLIYDTVKKHSWTPARFSGSLVGGKAALTLSGAQPEDEADYYCLLSFIDTRYPARYVFGTGTKVTVL

SEQ ID NO: 123 - protein, 248 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDDPWGSYRPRPFDYWGQGTLVTVSSGGGGSGGGGSGGGGSQSVLTQPPSASGTPGQRVTISCSGSSSNIGSNTVNWYQQLPGTTPKLLIYSNNQRPSGVPDRFSGSKSGTSASLAISGLQSEDEADYYCAAWDDSLNGWVFGGGTKLTVL

SEQ ID NO: 124 - protein, 247 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGASGYDWSLDYWGQGTLVTVSSGGGGSGGGGSGGGGSKIVMTQSPLSLPVTLGQPASISCRSSQSLVYSDGNTYLNWFQQRPGQSPRRLIYKVSNRHSGVPDRFSGSGSGTDFTLKISRVEAEDVGVYFCMQGSRWPPTFGPGTKVEIK

SEQ ID NO: 125 - protein, 244 aa, synthetic construct
QIQLVQSGTEVKKPGESLKISCKGSGYSFISHWIGWVRQMPGKGLEWMGIIYPGDSGTRYSPSFQGQATISADKSISTAYLQWSSLKASDTAMYYCARLADGGLQFDHWGQGTLVTVSSGGGGSGGGGSGGGGSQSALTQPASVSGSPGQSITISCTGTSSDVGGYNYVSWYQQHPGKAPKLMIYGVSNRPSGVSNRFSGSKSGNTASLTISGLQAEDEADYYCNSYTSSSTYVFGTGTKVTVL

SEQ ID NO: 126 - protein, 245 aa, synthetic construct
EVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARVPNYYDSSGTVWGQGTLVTVSSGGGGSGGGGSGGGGSNFMLTQPHSVSESPGKTVTISCTRSSGSIASNYVQWYQQRPGSAPTTVIYEDNQRPSGVPDRFSGSIDSSSNSASLTISGLEPEDEADYYCQSYDSTLLVFGGGTKLTVL

SEQ ID NO: 127 - protein, 500 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSLPVLTQPPSASGTPGQKVTISCSGSSSNIGSNYVFWYEQLPGAAPKLLMYSSNQRPSGVPDRFSGSKSGTSASLAISGLRSEDEADYYCAAWDDSLSGWVFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 495 aa, synthetic construct
QVQLVQSGGGVVQPGRSLRLSCAASGFTFSSYAMSWVRQAPGKGLEWVSAISGSGGNTNYADSVKGRFTISRDNSKNTLYLQMNSLRAEDTAVYYCAKGGSTSSGYRFDYWGQGTLVTVSSGGGGSGGGGSGGGGSQLVLTQSPSASASLGASVKLTCTLSSGHSNYAIAWHQQQPEKGPRFLMNVNRDGSYTKGDGIPDRFSGSSSGAERYLTISSLQSDDEADYYCQTWGTGVQVFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 491 aa, synthetic construct
QVQLQQSGAEVKKPGASVKVSCKASGYTFTSYAMHWVRQAPGQRLEWMGWINAGNGNTKYSQKLQGRVTITRDTSASTAYMELSSLRSEDTAVYYCARDSGYDLFDYWGQGTLVTVSSGGGGSGGGGSGGGGSQAVVTQEPSFSVSPGGTVTLTCGLTSGSVSTTYYPSWYQQTPGQTPRTLIYKTNLRSPGVPDRFSGSILGNKAALTITGAQADDDSDYYCLLYMGSGVWVFGGGTRLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 491 aa, synthetic construct
QVQLVQSGAEVKKPGESLKISCKGSGYSFTSYWIGWVRQMPGKGLEWMGIIYPGDSGTRYSPSFQGQVTISADKSISTAYLQWSSLKASNTAMYYCARQQGAGAFDIWGQGTMVTVSSGGGGSGGGGSGGGGSQSALTQPPSASGSPGQSVTISCTGTSRDVGGYNYVSWYQQHPGKAPKLILYEVTKRPSGVPDRFSGSKSGNTASLTISGLQAEDEADYYCSSYTSSSTVVFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 496 aa, synthetic construct
EVQLVQSGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPVVAGWYNWFDPWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGNTASLTVSGLQAGDEADYYCSSYAGSNNVVFGGGTKLAVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 496 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQTPSTLSASVGDRVTITCRASQTISNWLAWYQQKPGKAPKLLIYKASTLESGVPSRFSGTGSGTEFTLTISSLQPDDFATYFCQQYHSYSRTFGQGTKVDVTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 493 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYEYWGQGTLVTVSSGGGGSGGGGSGGGGSQAVVTQEPSLTVSPGETVTLTCGSSTGAVTSDHHPYWFQQKPGQVPRTLMHDTSTRYSWTPARFSGSIVGGKAALTLSGAQPEDEAEYHCFLYYSGTAIFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 495 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDYDFWSGSLGNYWGQGTLVTVSSGGGGSGGGGSGGGGSQSVLTQPPSASGTPGQRVTISCSGRSSNIGSNYVYWYQQPPGTAPKLLIYENNHRPSGVPDRFSASKSGTSASLAISGLRSEDEADYYCAAWDDTLNAWVFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 498 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQSPATLSLSPGERATLSCRASQSVGSSLAWFQQKPGQAPRLLIYDASNRATGIPARFSGSGSGTDFTLTISSLEPEDFAVYYCQQRSNWPPYTFGQGTKLEIKTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 499 aa, synthetic construct
QMQLVQSGGGVVQPGRSLRLSCAASGFTFSSYGMHWVRQAPGKGLERVAVISYDGSNKYYADSVKGRFTISRDNSKNTLYLQMNSLRAEDTALYYCAKDHHPYGSSDSFDYWGQGTLVTVSSGGGGSGGGGSGGGGSQAVVTQEPSLTVSPGGTVTLTCGSSTGAVTRGHYPYWLQQKPGQAPRTLIYDTVKKHSWTPARFSGSLVGGKAALTLSGAQPEDEADYYCLLSFIDTRYPARYVFGTGTKVTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 496 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDDPWGSYRPRPFDYWGQGTLVTVSSGGGGSGGGGSGGGGSQSVLTQPPSASGTPGQRVTISCSGSSSNIGSNTVNWYQQLPGTTPKLLIYSNNQRPSGVPDRFSGSKSGTSASLAISGLQSEDEADYYCAAWDDSLNGWVFGGGTKLTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 495 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGASGYDWSLDYWGQGTLVTVSSGGGGSGGGGSGGGGSKIVMTQSPLSLPVTLGQPASISCRSSQSLVYSDGNTYLNWFQQRPGQSPRRLIYKVSNRHSGVPDRFSGSGSGTDFTLKISRVEAEDVGVYFCMQGSRWPPTFGPGTKVEIKTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 492 aa, synthetic construct
QIQLVQSGTEVKKPGESLKISCKGSGYSFISHWIGWVRQMPGKGLEWMGIIYPGDSGTRYSPSFQGQATISADKSISTAYLQWSSLKASDTAMYYCARLADGGLQFDHWGQGTLVTVSSGGGGSGGGGSGGGGSQSALTQPASVSGSPGQSITISCTGTSSDVGGYNYVSWYQQHPGKAPKLMIYGVSNRPSGVSNRFSGSKSGNTASLTISGLQAEDEADYYCNSYTSSSTYVFGTGTKVTVLTRFVPVFLPAKPTTTPAPRPPTPAPTIASQPLSLRPEACRPAAGGAVHTRGLDFACDIYIWAPLAGTCGVLLLSLVITLYCNHRPPALEWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 673 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSLPVLTQPPSASGTPGQKVTISCSGSSSNIGSNYVFWYEQLPGAAPKLLMYSSNQRPSGVPDRFSGSKSGTSASLAISGLRSEDEADYYCAAWDDSLSGWVFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 668 aa, synthetic construct
QVQLVQSGGGVVQPGRSLRLSCAASGFTFSSYAMSWVRQAPGKGLEWVSAISGSGGNTNYADSVKGRFTISRDNSKNTLYLQMNSLRAEDTAVYYCAKGGSTSSGYRFDYWGQGTLVTVSSGGGGSGGGGSGGGGSQLVLTQSPSASASLGASVKLTCTLSSGHSNYAIAWHQQQPEKGPRFLMNVNRDGSYTKGDGIPDRFSGSSSGAERYLTISSLQSDDEADYYCQTWGTGVQVFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 664 aa, synthetic construct
QVQLQQSGAEVKKPGASVKVSCKASGYTFTSYAMHWVRQAPGQRLEWMGWINAGNGNTKYSQKLQGRVTITRDTSASTAYMELSSLRSEDTAVYYCARDSGYDLFDYWGQGTLVTVSSGGGGSGGGGSGGGGSQAVVTQEPSFSVSPGGTVTLTCGLTSGSVSTTYYPSWYQQTPGQTPRTLIYKTNLRSPGVPDRFSGSILGNKAALTITGAQADDDSDYYCLLYMGSGVWVFGGGTRLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 664 aa, synthetic construct
QVQLVQSGAEVKKPGESLKISCKGSGYSFTSYWIGWVRQMPGKGLEWMGIIYPGDSGTRYSPSFQGQVTISADKSISTAYLQWSSLKASNTAMYYCARQQGAGAFDIWGQGTMVTVSSGGGGSGGGGSGGGGSQSALTQPPSASGSPGQSVTISCTGTSRDVGGYNYVSWYQQHPGKAPKLILYEVTKRPSGVPDRFSGSKSGNTASLTISGLQAEDEADYYCSSYTSSSTVVFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 669 aa, synthetic construct
EVQLVQSGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPVVAGWYNWFDPWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGNTASLTVSGLQAGDEADYYCSSYAGSNNVVFGGGTKLAVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 670 aa, synthetic construct
PQVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQTPSTLSASVGDRVTITCRASQTISNWLAWYQQKPGKAPKLLIYKASTLESGVPSRFSGTGSGTEFTLTISSLQPDDFATYFCQQYHSYSRTFGQGTKVDVTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 666 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYEYWGQGTLVTVSSGGGGSGGGGSGGGGSQAVVTQEPSLTVSPGETVTLTCGSSTGAVTSDHHPYWFQQKPGQVPRTLMHDTSTRYSWTPARFSGSIVGGKAALTLSGAQPEDEAEYHCFLYYSGTAIFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 668 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDDYDFWSGSLGNYWGQGTLVTVSSGGGGSGGGGSGGGGSQSVLTQPPSASGTPGQRVTISCSGRSSNIGSNYVYWYQQPPGTAPKLLIYENNHRPSGVPDRFSASKSGTSASLAISGLRSEDEADYYCAAWDDTLNAWVFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 671 aa, synthetic construct
QVTLKESGGGLVQPGRSLRLSCAASGFTFDDYAMHWVRQAPGKGLEWVSGISWNSGSIGYADSVKGRFTISRDNAKNSLYLQMNSLRAEDTALYYCAKGANYDILTGYRKDNWFDPWGQGTLVTVSSGGGGSGGGGSGGGGSDIVMTQSPATLSLSPGERATLSCRASQSVGSSLAWFQQKPGQAPRLLIYDASNRATGIPARFSGSGSGTDFTLTISSLEPEDFAVYYCQQRSNWPPYTFGQGTKLEIKTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 672 aa, synthetic construct
QMQLVQSGGGVVQPGRSLRLSCAASGFTFSSYGMHWVRQAPGKGLERVAVISYDGSNKYYADSVKGRFTISRDNSKNTLYLQMNSLRAEDTALYYCAKDHHPYGSSDSFDYWGQGTLVTVSSGGGGSGGGGSGGGGSQAVVTQEPSLTVSPGGTVTLTCGSSTGAVTRGHYPYWLQQKPGQAPRTLIYDTVKKHSWTPARFSGSLVGGKAALTLSGAQPEDEADYYCLLSFIDTRYPARYVFGTGTKVTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 669 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDDPWGSYRPRPFDYWGQGTLVTVSSGGGGSGGGGSGGGGSQSVLTQPPSASGTPGQRVTISCSGSSSNIGSNTVNWYQQLPGTTPKLLIYSNNQRPSGVPDRFSGSKSGTSASLAISGLQSEDEADYYCAAWDDSLNGWVFGGGTKLTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 668 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGASGYDWSLDYWGQGTLVTVSSGGGGSGGGGSGGGGSKIVMTQSPLSLPVTLGQPASISCRSSQSLVYSDGNTYLNWFQQRPGQSPRRLIYKVSNRHSGVPDRFSGSGSGTDFTLKISRVEAEDVGVYFCMQGSRWPPTFGPGTKVEIKTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 665 aa, synthetic construct
QIQLVQSGTEVKKPGESLKISCKGSGYSFISHWIGWVRQMPGKGLEWMGIIYPGDSGTRYSPSFQGQATISADKSISTAYLQWSSLKASDTAMYYCARLADGGLQFDHWGQGTLVTVSSGGGGSGGGGSGGGGSQSALTQPASVSGSPGQSITISCTGTSSDVGGYNYVSWYQQHPGKAPKLMIYGVSNRPSGVSNRFSGSKSGNTASLTISGLQAEDEADYYCNSYTSSSTYVFGTGTKVTVLTREPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSASCTRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - DNA, 945 bp, synthetic construct
  source 1..945: mol_type = other DNA
atgaaatacctattgcctacggcagccgctggcttgctgctgctggcagctcagccggccatggcccaggtcaccttgaaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggctaattacgatattttgactggttatcggaaggataactggttcgacccctggggccagggaaccctggtcaccgtctcctcagggagtgcatccgccccaaagcttgaagaaggtgaattttcagaagcacgcgtactgcctgtgctgactcagcccccctcagcgtctgggacccccgggcagaaggtcaccatctcttgttctggaagcagctccaacatcggaagtaattatgtattctggtacgagcagctcccaggagcggcccccaagctcctcatgtatagcagtaatcagaggccctcaggggtccctgaccgattctctggctccaagtctggcacctcagcctccctggccatcagtgggctccggtccgaggatgaggctgactattattgtgcagcatgggatgacagcctgagtggttgggtgttcggcggagggaccaagctgaccgtcctaggtcagcccaaggctgccccctcggtcactctgttcccaccgtcctctgcggccgcaggttctgaacaaaagctgatctcagaagaagatctatcccatcatcaccatcatcat

SEQ ID NO: 154 - DNA, 930 bp, synthetic construct
  source 1..930: mol_type = other DNA
atgaaatacctattgcctacggcagccgctggcttgctgctgctggcagctcagccggccatggcccaggtccagctggtgcagtctgggggaggcgtggtccagcctgggaggtccctgagactctcctgtgcagcctctggattcacctttagcagctatgccatgagctgggtccgccaggctccagggaaggggctggagtgggtctcagctattagtggtagtggtggtaacacaaactacgcagactccgtgaagggccggttcaccatctccagagacaattccaagaacacgctgtatctgcaaatgaacagtctgagagccgaggacacggccgtatattactgtgcgaaaggggggagtactagtagtggttatcgttttgactactggggccagggaaccctggtcaccgtctcctcagggagtgcatccgccccaaagcttgaagaaggtgaattttcagaagcacgcgtacagcttgtgctgactcaatcgccctctgcctctgcctccctgggagcctcggtcaagctcacctgcactctgagcagtgggcacagcaactacgccatcgcatggcatcagcagcagccagagaagggccctcggttcttgatgaacgttaatcgtgatggcagctacaccaagggggacgggatccctgatcgcttctcaggctccagctctggggctgagcgctacctcaccatctccagcctccagtctgacgatgaggctgactattactgtcagacctggggcactggcgttcaagtgttcggcggagggaccaagctgaccgtcctaagtcagcccaaggctgccccctcggtcactctgttcccgccctcctctgcggccgcaggttctgaacaaaagctgatctcagaagaagatctatcccatcatcaccatcatcat

SEQ ID NO: 155 - DNA, 918 bp, synthetic construct
  source 1..918: mol_type = other DNA
atgaaatacctattgcctacggcagccgctggcttgctgctgctggcagctcagccggccatggcccaggtacagctgcagcagtcaggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagctccagggcagagtcaccattaccagggacacatccgcgagcacagcctacatggagctgagcagcctgagatctgaagacacggctgtgtattactgtgcgagagatagtggctacgacctctttgactactggggccagggaaccctggtcaccgtctcctcagggagtgcatccgccccaaagcttgaagaaggtgaattttcagaagcacgcgtacaggctgtggtgactcaggagccatcgttctcagtgtcccctggagggacagtcacacttacttgtggcttgacctctggctcagtctctactacttactaccccagctggtaccagcagaccccaggccagactccacgcacactcatctacaaaacaaatcttcgctctcctggggtccctgatcgcttctctggctccatccttgggaacaaagctgccctcaccattacgggggcccaggcagacgacgactctgattactactgtctgctgtatatgggtagtggcgtttgggtgttcggcggagggaccaggttgaccgtcctgggtcagcccaaggctgccccctcggtcactctgttcccgccctcctctgcggccgcaggttctgaacaaaagctgatctcagaagaagatctatcccatcatcaccatcatcat

SEQ ID NO: 156 - DNA, 918 bp, synthetic construct
  source 1..918: mol_type = other DNA
atgaaatacctattgcctacggcagccgctggcttgctgctgctggcagctcagccggccatggcccaagtgcagctggtgcagtctggagcagaggtgaaaaagccgggggagtctctgaagatctcctgtaagggttctggatacagctttaccagctactggatcggctgggtgcgccagatgcccgggaaaggcctggagtggatggggatcatctatcctggtgactctggtaccagatacagcccgtccttccaaggccaggtcaccatctcagccgacaagtccatcagcaccgcctacctgcagtggagcagcctgaaggcctcgaacaccgccatgtattactgtgcgagacaacagggcgccggtgcttttgatatctggggccaagggacaatggtcaccgtctcttcagggagtgcatccgccccaaagcttgaagaaggtgaattttcagaagcacgcgtacagtctgccctgactcagcctccctccgcgtccgggtctcctggacagtcagtcaccatctcctgcactggaaccagccgtgacgttggtggttataactatgtctcctggtaccaacaacaccccggcaaagcccccaaactcatactttatgaggtcactaagcggccctcaggggtccctgatcgcttctctggctccaagtctggcaacacggcctccctgaccatctctgggctccaggctgaggacgaggctgattattactgcagctcatatacaagcagcagcaccgtggtgttcggcggagggaccaagctgaccgtcctaggtcagcccaaggctgccccctcggtcactctgttcccgccgtcctctgcggccgcaggttctgaacaaaagctgatctcagaagaagatctatcccatcatcaccatcatcat

SEQ ID NO: 157 - DNA, 933 bp, synthetic construct
  source 1..933: mol_type = other DNA
atgaaatacctattgcctacggcagccgctggcttgctgctgctggcagctcagccggccatggccgaagtgcagctggtgcagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagagtcccggtggtagctgggtggtacaactggttcgacccctggggccagggaaccctggtcaccgtctcctcagggagtgcatccgccccaaagcttgaagaaggtgaattttcagaagcacgcgtacagtctgtgctgacgcagccgccctcagcgtctgggacccccgggcagagggtcaccatctcttgttctggaagcagctccaacatcgggagtaattatgtatactggtaccagcagctcccaggaacggcccccaaactcctcatctataggaataatcagcggccctcaggggtccctgatcgcttctctggctccaagtctggcaacacggcctccctgaccgtctctgggctccaggctggggatgaggccgattattactgcagctcatatgcaggcagcaacaatgtggtattcggcggagggaccaagctggccgtcctaggtcagcccaaggctgccccctcggtcactctgttcccaccgtcctctgcggccgcaggttctgaacaaaagctgatctcagaagaagatctatcccatcatcaccatcatcat

SEQ ID NO: 158 - DNA, 912 bp, synthetic construct
  source 1..912: mol_type = other DNA
atgaaatacctattgcctacggcagccgctggcttgctgctgctggcagctcagccggccatggcccaggttaccttgaaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggctaattacgatattttgactggttatcggaaggataactggttcgacccctggggccagggaaccctggtcaccgtctcctcagggagtgcatccgccccaaagcttgaagaaggtgaattttcagaagcacgcgtagatattgtgatgactcagactccttccaccctgtctgcatctgtgggagacagagtcaccatcacttgccgggccagtcagactattagtaactggttggcctggtatcagcagaaaccagggaaagcccctaaactcctaatttataaggcgtctactttagaaagtggggtcccatcaaggttcagcggcactggatctgggacagaattcactctcaccatcagcagcctgcagcctgatgattttgcaacttatttttgccaacaatatcatagttattctcggacgttcggccaagggaccaaggtcgatgtcgcacgaactgtggctgcaccatctgtcgcggccgcaggttctgaacaaaagctgatctcagaagaagatctatcccatcatcaccatcatcat

SEQ ID NO: 159 - DNA, 924 bp, synthetic construct
  source 1..924: mol_type = other DNA
atgaaatacctattgcctacggcagccgctggcttgctgctgctggcagctcagccggccatggcccaggttcagctggtgcagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggccaattacgatattttgactggttatgagtactggggccagggaaccctggtcaccgtctcctcagggagtgcatccgccccaaagcttgaagaaggtgaattttcagaagcacgcgtacaggctgtggtgactcaggagccctcactgactgtgtccccaggagagacagtcactctcacctgtggctccagtactggagctgtcaccagtgatcatcatccctactggttccaacagaagcctggccaagtccccaggacactgatgcatgatacaagcaccagatactcctggacccctgcccggttctcaggctccatcgttgggggcaaagctgccctgactctttcgggtgcgcagcctgaggatgaggctgaatatcactgtttcctctattacagtggaactgcgatattcggcggagggaccaagctgaccgtcctaagtcagcccaaggctgccccctcggtcactctgttcccgccgtcctctgcggccgcaggttctgaacaaaagctgatctcagaagaagatctatcccatcatcaccatcatcat

SEQ ID NO: 160 - DNA, 930 bp, synthetic construct
  source 1..930: mol_type = other DNA
atgaaatacctattgcctacggcagccgctggcttgctgctgctggcagctcagccggccatggccgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgattacgatttttggagtggttccctagggaactactggggccagggaaccctggtcaccgtctcctcagggagtgcatccgccccaaagcttgaagaaggtgaattttcagaagcacgcgtacagtctgtgctgacgcagccgccctcagcgtctgggacccccgggcagagggtcaccatctcttgttctggacgcagctccaacatcggaagtaattatgtatactggtaccagcagcccccaggaacggcccccaaactcctcatctatgagaataatcaccggccctcaggggtccctgaccgattctctgcctccaagtctggcacctcagcctccctggccatcagtgggctccggtccgaggatgaggctgattattactgtgcagcatgggacgacaccttgaatgcttgggtgttcggcggagggaccaagctgaccgtcctaggtcagcccaaggctgccccctcggtcactctgttcccgccgtcctctgcggccgcaggttctgaacaaaagctgatctcagaagaagatctatcccatcatcaccatcatcat

SEQ ID NO: 161 - DNA, 915 bp, synthetic construct
  source 1..915: mol_type = other DNA
atgaaatacctattgcctacggcagccgctggcttgctgctgctggcagctcagccggccatggcccaggtcaccttgaaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggctaattacgatattttgactggttatcggaaggataactggttcgacccctggggccagggaaccctggtcaccgtctcctcagggagtgcatccgccccaaagcttgaagaaggtgaattttcagaagcacgcgtagatattgtgatgacacagtctccagccaccctgtctttgtctccaggggaaagagccaccctctcctgcagggccagtcagagtgttggcagctccttagcctggttccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccctacacttttggccaggggaccaaactggagatcaaacgaactgtggctgcaccatctgtcgcggccgcaggttctgaacaaaagctgatctcagaagaagatctatcccatcatcaccatcatcat

SEQ ID NO: 162 - DNA, 942 bp, synthetic construct
  source 1..942: mol_type = other DNA
atgaaatacctattgcctacggcagccgctggcttgctgctgctggcagctcagccggccatggcccagatgcagctggtgcagtctgggggaggcgtggtccagcctgggaggtccctgagactctcctgtgcagcctctggattcaccttcagtagctatggcatgcactgggtccgccaggctccaggcaaggggctggagcgggtggcagttatatcatatgatggaagtaataaatactatgcagactccgtgaagggccgattcaccatctccagagacaattccaagaacacgctgtatctgcaaatgaacagcctgagagctgaggacacggctctgtattactgtgcgaaagaccaccatccgtatggaagtagtgactcttttgactactggggccagggaaccctggtcaccgtctcctcagggagtgcatccgccccaaagcttgaagaaggtgaattttcagaagcacgcgtacaggctgtggtgactcaggagccctcactgactgtgtccccaggagggacagtcactctcacctgtggctccagcactggggctgtcaccaggggtcattatccctattggttgcagcagaagcctggccaagcccccaggacactgatttatgatacggtcaagaaacactcctggacccctgcccggttctctggctccctcgttgggggcaaagctgccctgaccctttcgggtgcgcagcctgaagacgaggctgactattattgcttgctctcctttattgatactcggtaccctgctcggtatgtcttcggaaccgggaccaaggtcaccgtcctaggtcagcccaaggccaaccccactgtcactctgttcccgccctcctctgcggccgcaggttctgaacaaaagctgatctcagaagaagatctatcccatcatcaccatcatcat

SEQ ID NO: 163 - DNA, 933 bp, synthetic construct
  source 1..933: mol_type = other DNA
atgaaatacctattgcctacggcagccgctggcttgctgctgctggcagctcagccggccatggcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatgatccgtggggttcctatcggccccgtccgtttgactactggggccagggaaccctggtcaccgtctcctcagggagtgcatccgccccaaagcttgaagaaggtgaattttcagaagcacgcgtacagtctgtgctgacgcagccgccctcagcgtctgggacccccgggcagagggtcaccatctcttgttctggaagcagctccaacatcggaagtaatactgtaaactggtaccagcagctcccaggaacgacccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggtcagcccaaggctgccccctcggtcactctgttcccgccgtcctctgcggccgcaggttctgaacaaaagctgatctcagaagaagatctatcccatcatcaccatcatcat

SEQ ID NO: 164 - DNA, 906 bp, synthetic construct
  source 1..906: mol_type = other DNA
atgaaatacctattgcctacggcagccgctggcttgctgctgctggcagctcagccggccatggccgaagtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggagcaagtggatatgactggtcccttgactactggggccagggaaccctggtcaccgtctcctcagggagtgcatccgccccaaagcttgaagaaggtgaattttcagaagcacgcgtaaaaattgtgatgacacagtctccactctccctgcccgtcacccttggacagccggcctccatctcctgcaggtctagtcaaagcctcgtatacagtgatggaaacacctacttgaattggtttcagcagaggccaggccaatctccaaggcgcctcatttataaggtttctaaccggcactctggggtcccagacagattcagcggcagtgggtcaggcactgatttcacactgaaaatcagcagggtggaggctgaggatgttggggtttatttctgcatgcaaggttcacgctggcctccgacgttcggcccagggaccaaggtggaaatcaaacgaactgtggctgcaccatctgtcgcggccgcaggttctgaacaaaagctgatctcagaagaagatctatcccatcatcaccatcatcat

SEQ ID NO: 165 - DNA, 927 bp, synthetic construct
  source 1..927: mol_type = other DNA
atgaaatacctattgcctacggcagccgctggcttgctgctgctggcagctcagccggccatggccatggcccaaatccagctggtgcagtctggaacagaggtgaaaaagcccggggagtctctgaagatctcctgtaagggttctggatacagctttatcagtcactggatcggctgggtgcgccagatgcccgggaaaggcctggagtggatggggatcatctatcctggtgactctggtaccagatacagcccgtccttccaaggccaggccaccatctcagccgacaagtccatcagcaccgcctacctgcagtggagcagcctgaaggcctcggacaccgccatgtattactgtgcgagactcgctgacggaggccttcaatttgaccactggggccagggaaccctggtcaccgtctcctcagggagtgcatccgccccaaagcttgaagaaggtgaattttcagaagcacgcgtacagtctgccctgactcagcctgcctccgtgtctgggtctcctggacagtcgatcaccatctcctgcactggaaccagcagtgacgttggtggttacaactatgtctcctggtaccaacagcacccaggcaaagcccccaaactcatgatttatggggtcagtaatcggccctcaggggtttctaatcgcttctctggctccaagtctggcaacacggcctccctgaccatctctgggctccaggctgaggacgaggctgattattactgcaactcatatacaagcagcagcacttatgtcttcggaactgggaccaaggtcaccgtcctaggtcagcccaaggccaaccccactgtcactctgttcccaccgtcctctgcggccgcaggttctgaacaaaagctgatctcagaagaagatctatcccatcatcaccatcatcat

SEQ ID NO: 166 - DNA, 924 bp, synthetic construct
  source 1..924: mol_type = other DNA
atgaaatacctattgcctacggcagccgctggcttgctgctgctggcagctcagccggccatggccgaggtgcagctggtgcagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagtaccaaattactatgatagtagtggcaccgtgtggggccagggaaccctggtcaccgtctcctcagggagtgcatccgccccaaagcttgaagaaggtgaattttcagaagcacgcgtaaattttatgctgactcagccccactctgtgtcggagtctccggggaagacggtaaccatctcctgcacccgcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactggagcctgaggacgaggctgactactactgtcagtcttatgatagcaccttattggtgttcggcggcgggaccaaactgactgtcctaggtcagcccaaggctgccccctcggtcactctgttcccaccgtcctctgcggccgcaggttctgaacaaaagctgatctcagaagaagatctatcccatcatcaccatcatcat

SEQ ID NO: 167 - DNA, 382 bp, synthetic construct
  source 1..382: mol_type = other DNA
caggtcaccttgaaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggctaattacgatattttgactggttatcggaaggataactggttcgacccctggggccagggaaccctggtcaccgtctcctcag

SEQ ID NO: 168 - DNA, 331 bp, synthetic construct
  source 1..331: mol_type = other DNA
ctgcctgtgctgactcagcccccctcagcgtctgggacccccgggcagaaggtcaccatctcttgttctggaagcagctccaacatcggaagtaattatgtattctggtacgagcagctcccaggagcggcccccaagctcctcatgtatagcagtaatcagaggccctcaggggtccctgaccgattctctggctccaagtctggcacctcagcctccctggccatcagtgggctccggtccgaggatgaggctgactattattgtgcagcatgggatgacagcctgagtggttgggtgttcggcggagggaccaagctgaccgtcctag

SEQ ID NO: 169 - DNA, 364 bp, synthetic construct
  source 1..364: mol_type = other DNA
caggtccagctggtgcagtctgggggaggcgtggtccagcctgggaggtccctgagactctcctgtgcagcctctggattcacctttagcagctatgccatgagctgggtccgccaggctccagggaaggggctggagtgggtctcagctattagtggtagtggtggtaacacaaactacgcagactccgtgaagggccggttcaccatctccagagacaattccaagaacacgctgtatctgcaaatgaacagtctgagagccgaggacacggccgtatattactgtgcgaaaggggggagtactagtagtggttatcgttttgactactggggccagggaaccctggtcaccgtctcctcag

SEQ ID NO: 170 - DNA, 333 bp, synthetic construct
  source 1..333: mol_type = other DNA
cagcttgtgctgactcaatcgccctctgcctctgcctccctgggagcctcggtcaagctcacctgcactctgagcagtgggcacagcaactacgccatcgcatggcatcagcagcagccagagaagggccctcggttcttgatgaacgttaatcgtgatggcagctacaccaagggggacgggatccctgatcgcttctcaggctccagctctggggctgagcgctacctcaccatctccagcctccagtctgacgatgaggctgactattactgtcagacctggggcactggcgttcaagtgttcggcggagggaccaagctgaccgtccta

SEQ ID NO: 171 - DNA, 355 bp, synthetic construct
  source 1..355: mol_type = other DNA
caggtacagctgcagcagtcaggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagctccagggcagagtcaccattaccagggacacatccgcgagcacagcctacatggagctgagcagcctgagatctgaagacacggctgtgtattactgtgcgagagatagtggctacgacctctttgactactggggccagggaaccctggtcaccgtctcctcag

SEQ ID NO: 172 - DNA, 331 bp, synthetic construct
  source 1..331: mol_type = other DNA
caggctgtggtgactcaggagccatcgttctcagtgtcccctggagggacagtcacacttacttgtggcttgacctctggctcagtctctactacttactaccccagctggtaccagcagaccccaggccagactccacgcacactcatctacaaaacaaatcttcgctctcctggggtccctgatcgcttctctggctccatccttgggaacaaagctgccctcaccattacgggggcccaggcagacgacgactctgattactactgtctgctgtatatgggtagtggcgtttgggtgttcggcggagggaccaggttgaccgtcctgg

SEQ ID NO: 173 - DNA, 355 bp, synthetic construct
  source 1..355: mol_type = other DNA
caagtgcagctggtgcagtctggagcagaggtgaaaaagccgggggagtctctgaagatctcctgtaagggttctggatacagctttaccagctactggatcggctgggtgcgccagatgcccgggaaaggcctggagtggatggggatcatctatcctggtgactctggtaccagatacagcccgtccttccaaggccaggtcaccatctcagccgacaagtccatcagcaccgcctacctgcagtggagcagcctgaaggcctcgaacaccgccatgtattactgtgcgagacaacagggcgccggtgcttttgatatctggggccaagggacaatggtcaccgtctcttcag

SEQ ID NO: 174 - DNA, 331 bp, synthetic construct
  source 1..331: mol_type = other DNA
cagtctgccctgactcagcctccctccgcgtccgggtctcctggacagtcagtcaccatctcctgcactggaaccagccgtgacgttggtggttataactatgtctcctggtaccaacaacaccccggcaaagcccccaaactcatactttatgaggtcactaagcggccctcaggggtccctgatcgcttctctggctccaagtctggcaacacggcctccctgaccatctctgggctccaggctgaggacgaggctgattattactgcagctcatatacaagcagcagcaccgtggtgttcggcggagggaccaagctgaccgtcctag

SEQ ID NO: 175 - DNA, 373 bp, synthetic construct
  source 1..373: mol_type = other DNA
gaagtgcagctggtgcagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagagtcccggtggtagctgggtggtacaactggttcgacccctggggccagggaaccctggtcaccgtctcctcag

SEQ ID NO: 176 - DNA, 328 bp, synthetic construct
  source 1..328: mol_type = other DNA
cagtctgtgctgacgcagccgccctcagcgtctgggacccccgggcagagggtcaccatctcttgttctggaagcagctccaacatcgggagtaattatgtatactggtaccagcagctcccaggaacggcccccaaactcctcatctataggaataatcagcggccctcaggggtccctgatcgcttctctggctccaagtctggcaacacggcctccctgaccgtctctgggctccaggctggggatgaggccgattattactgcagctcatatgcaggcagcaacaatgtggtattcggcggagggaccaagctggccgtcctag

SEQ ID NO: 177 - DNA, 382 bp, synthetic construct
  source 1..382: mol_type = other DNA
caggttaccttgaaggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggctaattacgatattttgactggttatcggaaggataactggttcgacccctggggccagggaaccctggtcaccgtctcctcag

SEQ ID NO: 178 - DNA, 318 bp, synthetic construct
  source 1..318: mol_type = other DNA
gatattgtgatgactcagactccttccaccctgtctgcatctgtgggagacagagtcaccatcacttgccgggccagtcagactattagtaactggttggcctggtatcagcagaaaccagggaaagcccctaaactcctaatttataaggcgtctactttagaaagtggggtcccatcaaggttcagcggcactggatctgggacagaattcactctcaccatcagcagcctgcagcctgatgattttgcaacttatttttgccaacaatatcatagttattctcggacgttcggccaagggaccaaggtcgatgtc

SEQ ID NO: 179 - DNA, 364 bp, synthetic construct
  source 1..364: mol_type = other DNA
caggttcagctggtgcagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggggccaattacgatattttgactggttatgagtactggggccagggaaccctggtcaccgtctcctcag

SEQ ID NO: 180 - DNA, 327 bp, synthetic construct
  source 1..327: mol_type = other DNA
caggctgtggtgactcaggagccctcactgactgtgtccccaggagagacagtcactctcacctgtggctccagtactggagctgtcaccagtgatcatcatccctactggttccaacagaagcctggccaagtccccaggacactgatgcatgatacaagcaccagatactcctggacccctgcccggttctcaggctccatcgttgggggcaaagctgccctgactctttcgggtgcgcagcctgaggatgaggctgaatatcactgtttcctctattacagtggaactgcgatattcggcggagggaccaagctgaccgtccta

SEQ ID NO: 181 - DNA, 350 bp, synthetic construct
  source 1..350: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgattacgatttttggagtggttccctagggaactactggggccagggaaccct

SEQ ID NO: 182 - DNA, 331 bp, synthetic construct
  source 1..331: mol_type = other DNA
cagtctgtgctgacgcagccgccctcagcgtctgggacccccgggcagagggtcaccatctcttgttctggacgcagctccaacatcggaagtaattatgtatactggtaccagcagcccccaggaacggcccccaaactcctcatctatgagaataatcaccggccctcaggggtccctgaccgattctctgcctccaagtctggcacctcagcctccctggccatcagtgggctccggtccgaggatgaggctgattattactgtgcagcatgggacgacaccttgaatgcttgggtgttcggcggagggaccaagctgaccgtcctag

SEQ ID NO: 183 - DNA, 382 bp, synthetic construct; identical to SEQ ID NO: 167
  source 1..382: mol_type = other DNA

SEQ ID NO: 184 - DNA, 325 bp, synthetic construct
  source 1..325: mol_type = other DNA
gatattgtgatgacacagtctccagccaccctgtctttgtctccaggggaaagagccaccctctcctgcagggccagtcagagtgttggcagctccttagcctggttccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccctacacttttggccaggggaccaaactggagatcaaac

SEQ ID NO: 185 - DNA, 367 bp, synthetic construct
  source 1..367: mol_type = other DNA
cagatgcagctggtgcagtctgggggaggcgtggtccagcctgggaggtccctgagactctcctgtgcagcctctggattcaccttcagtagctatggcatgcactgggtccgccaggctccaggcaaggggctggagcgggtggcagttatatcatatgatggaagtaataaatactatgcagactccgtgaagggccgattcaccatctccagagacaattccaagaacacgctgtatctgcaaatgaacagcctgagagctgaggacacggctctgtattactgtgcgaaagaccaccatccgtatggaagtagtgactcttttgactactggggccagggaaccctggtcaccgtctcctcag

SEQ ID NO: 186 - DNA, 343 bp, synthetic construct
  source 1..343: mol_type = other DNA
caggctgtggtgactcaggagccctcactgactgtgtccccaggagggacagtcactctcacctgtggctccagcactggggctgtcaccaggggtcattatccctattggttgcagcagaagcctggccaagcccccaggacactgatttatgatacggtcaagaaacactcctggacccctgcccggttctctggctccctcgttgggggcaaagctgccctgaccctttcgggtgcgcagcctgaagacgaggctgactattattgcttgctctcctttattgatactcggtaccctgctcggtatgtcttcggaaccgggaccaaggtcaccgtcctag

SEQ ID NO: 187 - DNA, 370 bp, synthetic construct
  source 1..370: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatgatccgtggggttcctatcggccccgtccgtttgactactggggccagggaaccctggtcaccgtctcctcag

SEQ ID NO: 188 - DNA, 331 bp, synthetic construct
  source 1..331: mol_type = other DNA
cagtctgtgctgacgcagccgccctcagcgtctgggacccccgggcagagggtcaccatctcttgttctggaagcagctccaacatcggaagtaatactgtaaactggtaccagcagctcccaggaacgacccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctag

SEQ ID NO: 189 - DNA, 361 bp, synthetic construct
  source 1..361: mol_type = other DNA
gaagtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggagcaagtggatatgactggtcccttgactactggggccagggaaccctggtcaccgtctcctcag

SEQ ID NO: 190 - DNA, 337 bp, synthetic construct
  source 1..337: mol_type = other DNA
aaaattgtgatgacacagtctccactctccctgcccgtcacccttggacagccggcctccatctcctgcaggtctagtcaaagcctcgtatacagtgatggaaacacctacttgaattggtttcagcagaggccaggccaatctccaaggcgcctcatttataaggtttctaaccggcactctggggtcccagacagattcagcggcagtgggtcaggcactgatttcacactgaaaatcagcagggtggaggctgaggatgttggggtttatttctgcatgcaaggttcacgctggcctccgacgttcggcccagggaccaaggtggaaatcaaac

SEQ ID NO: 191 - DNA, 358 bp, synthetic construct
  source 1..358: mol_type = other DNA
caaatccagctggtgcagtctggaacagaggtgaaaaagcccggggagtctctgaagatctcctgtaagggttctggatacagctttatcagtcactggatcggctgggtgcgccagatgcccgggaaaggcctggagtggatggggatcatctatcctggtgactctggtaccagatacagcccgtccttccaaggccaggccaccatctcagccgacaagtccatcagcaccgcctacctgcagtggagcagcctgaaggcctcggacaccgccatgtattactgtgcgagactcgctgacggaggccttcaatttgaccactggggccagggaaccctggtcaccgtctcctcag

SEQ ID NO: 192 - DNA, 331 bp, synthetic construct
  source 1..331: mol_type = other DNA
cagtctgccctgactcagcctgcctccgtgtctgggtctcctggacagtcgatcaccatctcctgcactggaaccagcagtgacgttggtggttacaactatgtctcctggtaccaacagcacccaggcaaagcccccaaactcatgatttatggggtcagtaatcggccctcaggggtttctaatcgcttctctggctccaagtctggcaacacggcctccctgaccatctctgggctccaggctgaggacgaggctgattattactgcaactcatatacaagcagcagcacttatgtcttcggaactgggaccaaggtcaccgtcctag

SEQ ID NO: 193 - DNA, 361 bp, synthetic construct
  source 1..361: mol_type = other DNA
gaggtgcagctggtgcagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagtaccaaattactatgatagtagtggcaccgtgtggggccagggaaccctggtcaccgtctcctcag

SEQ ID NO: 194 - DNA, 331 bp, synthetic construct
  source 1..331: mol_type = other DNA
aattttatgctgactcagccccactctgtgtcggagtctccggggaagacggtaaccatctcctgcacccgcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactggagcctgaggacgaggctgactactactgtcagtcttatgatagcaccttattggtgttcggcggcgggaccaaactgactgtcctag

SEQ ID NO: 195 - DNA, 511 bp, Homo sapiens
  source 1..511: mol_type = genomic DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 196 - DNA, 118 bp, synthetic construct
  source 1..118: mol_type = other DNA
acccctctctccagccactaagccagttgctccctcggctgacggctgcacgcgaggcctccgaacgtcttacgccttgtggcgcgcccgtccttgtcccgggtgtgatggcggggtg

SEQ ID NO: 197 - DNA, 221 bp, synthetic construct
  source 1..221: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 198 - DNA, 324 bp, synthetic construct
  source 1..324: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 199 - DNA, 422 bp, synthetic construct
  source 1..422: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 200 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 201 - protein, 362 aa, synthetic construct
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 202 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 203 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 204 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 205 - protein, 441 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 206 - DNA, 1296 bp, synthetic construct
  source 1..1296: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 207 - DNA, 1352 bp, synthetic construct
  source 1..1352: mol_type = other DNA
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 208 - protein, 19 aa, synthetic construct
ATNFSLLKQAGDVEENPGP

SEQ ID NO: 209 - protein, 18 aa, synthetic construct
EGRGSLLTCGDVEENPGP

SEQ ID NO: 210 - protein, 20 aa, synthetic construct
QCTNYALLKLAGDVESNPGP

SEQ ID NO: 211 - protein, 22 aa, synthetic construct
VKQTLNFDLLKLAGDVESNPGP

SEQ ID NO: 212 - skipped

SEQ ID NO: 213 - protein, 4 aa, synthetic construct
RRKR

SEQ ID NO: 214 - DNA, 3443 bp, synthetic construct
  source 1..3443: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagtatctgcggccgcataggatccgccaccatggccctgcccgttaccgcgttactgctacccctggccctcctgctccatgccgctagaccccaagtgcagctggtgcaatccggaggaggcgtggtacagcccggcagaagcctgagactgagctgcgccgcaagcggcttcaccttcagcagctacgccatgagctgggtgagacaagcccccggcaagggcctggagtgggtgagcgccatcagtgggagcggcggcaacacaaactacgccgacagcgtgaagggcagattcaccatcagcagagacaacagcaagaacaccctgtacctgcagatgaacagcctgagagccgaggacaccgccgtgtactactgcgccaagggcggcagcacaagcagcggctacagattcgactattggggccaaggcaccctggttaccgttagctcggggggaggtgggagtggaggcggtggttccggcgggggcggcagtcagctggttctgacgcaaagccctagcgcaagcgcaagcctgggcgcaagcgtgaagctgacctgcaccctgagcagcggccacagcaactacgccatcgcctggcatcagcagcagcccgagaagggccctagattcctgatgaacgtgaacagagacggcagctacaccaagggcgacggcatccccgacagattcagcggcagcagcagcggcgccgagagatacctgaccatcagcagcctgcagagcgacgacgaggccgactactactgtcagacctggggcaccggcgtgcaagtgttcggcggcggcaccaagctgaccgtgctgacgcgtttcgtgcccgtgttcctgcctgccaagccaacaacaacccctgctccaaggccaccaactccagcacctaccattgcctctcaacccctgagcctcagacctgaggcatgtagaccagccgcaggcggcgctgttcacacaagaggccttgacttcgcctgtgatatctatatttgggctcccctggccggcacttgcggagttttgctgctgtctctcgtgatcacactctactgcaatcacagaccaccagccctcgagtgggtccgaagcaagagaagcagactgctgcacagcgactacatgaacatgacccctagacggcccggacctaccagaaagcactaccagccttacgctcctcctcgggactttgccgcctatcggagcgctagctgtacaagagtgaagttcagcagatccgccgatgctcccgcctatcagcagggacagaatcagctgtataacgagctgaacctggggcgcagagaagagtacgacgtgctggacaagagaagaggcagggaccctgagatgggcggcaagcccagaagaaagaaccctcaagagggcctctacaacgagctgcagaaagacaagatggccgaggcctacagcgagatcggaatgaagggcgaacgcagaagaggaaagggccacgacggactgtatcagggcctgagcacagccaccaaggatacctatgacgccctgcacatgcaggccctgcctccaagatctcagctcgaaggcagaggctccctgctgacatgcggagatgtggaagaaaatcccggaccaatgcctaatcctcggcctggcaagcctagcgctccttctcttgctctgggaccttctcctggcgcctctccatcttggagagccgctcctaaagccagcgatctgcttggagctagaggccctggcggcacatttcagggcagagatcttagaggcggagcccacgctagctcctccagccttaatcctatgcctcctagccagctccagctgccaacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaaagccccgccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaaatggcctggctgcgagaaggtgttcgaggaacccgaggacttcctgaagcactgtcaggccgaccatctgctggacgagaaaggcagagcccagtgtctcctgcagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgttgtgccagcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagaggaccctgaacgagatctaccactggtttacccggatgttcgccttcttccggaatcaccctgccacctggaagaacgccatccggcataatctgagcctgcacaagtgcttcgtgcgcgtggaatctgagaaaggcgccgtgtggacagtggacgagctggaattcagaaagaagcggagccagcggcctagcagatgctctaaccctacacctggacct

SEQ ID NO: 215 - DNA, 407 bp, synthetic construct
  source 1..407: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccgg

SEQ ID NO: 216 - DNA, 1182 bp, Homo sapiens
  source 1..1182: mol_type = genomic DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 217 - DNA, 1576 bp, Homo sapiens
  source 1..1576: mol_type = genomic DNA
cccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctccagtacgtgattcttgatcccgagcttcgggttggaagtgggtgggagagttcgaggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaagatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaaaactacccctaaaat

SEQ ID NO: 218 - protein, 529 aa, Homo sapiens
MFTVLTRQPCEQAGLKALYRTPTIIALVVLLVSIVVLVSITVIQIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPVFVGTLAFFTAAALLCLAFLAYLCSATRRKRHSEHAFDHAVDSD

SEQ ID NO: 219 - protein, 529 aa, Mus sp.
MFTVMTRQPCEQAGFRALSRTPAIVTLVVLLVSIVVLVTLTLIQIRHPQVLPPGLKYGVVLDAGSSRTTVYVYQWPAEKENNTGVVSQTFRCSVKGSGISSYENNPQDAPKAFEDCILKVKEQVPEHLHGSTRIYLGATAGMRLLRLQNETAAREVLESIQSYFKSQPFDFRGAQIISGQEEGVYGWITANYIMGNFLEKNLWHMWVHPHGVDTTGALDLGGASTQISFVAGEKMEPNASDTVQVSLYGYTYTLYTHSFQCYGQNEAEKKFLAMLLQSPSTEANISNPCYPQGYSTAFTLGHVFGSLCTEKQRPESYNSSKSVTFMGTGDPRLCREKVASVFDFNACQEQDACSFDGIYQPKVQGPFVAFAGFYYTASALNLSGSFSLTSFNDSSWDFCRHTWSELPALLSRFDETYARSYCFSAHYIYHLLVNGYKFTEETWPQIRFEKEVGNSSIAWSLGYMLSLTNQIPAGSPLIHLPIQPPVFMGVLAFFTAIALLCLAFLLYLCSSFRTKERSENAFDQAVDSD

SEQ ID NO: 220 - protein, 4 aa, synthetic construct
GGGS

SEQ ID NO: 221 - protein, 10 aa, synthetic construct
ETSGGGGSRL

SEQ ID NO: 222 - protein, 16 aa, synthetic construct
SGGGGSGGGGSGGGGS

SEQ ID NO: 223 - protein, 5 aa, synthetic construct
GGGGS

SEQ ID NO: 224 - protein, 6 aa, synthetic construct
GGGGGS

SEQ ID NO: 225 - protein, 7 aa, synthetic construct
GGGGGGS

SEQ ID NO: 226 - protein, 15 aa, synthetic construct
GGGGSGGGGSGGGGS

SEQ ID NO: 227 - protein, 5 aa, synthetic construct
GGGGG

SEQ ID NO: 228 - protein, 10 aa, synthetic construct
GGGGSGGGGS

SEQ ID NO: 229 - protein, 20 aa, synthetic construct
GGGGSGGGGSGGGGSGGGGS

SEQ ID NO: 230 - protein, 7 aa, synthetic construct
GGGGGGG

SEQ ID NO: 231 - protein, 6 aa, synthetic construct
GGGGGG

SEQ ID NO: 232 - protein, 8 aa, synthetic construct
GGGGGGGG

SEQ ID NO: 233 - protein, 18 aa, synthetic construct
KESGSVSSEQLAQFRSLD

SEQ ID NO: 234 - protein, 14 aa, synthetic construct
EGKSSGSGSESKST

SEQ ID NO: 235 - protein, 12 aa, synthetic construct
GSAGSAAGSGEF

SEQ ID NO: 236 - protein, 16 aa, synthetic construct
SGGGGSAGSAAGSGEF

SEQ ID NO: 237 - protein, 16 aa, synthetic construct
SGGGLLLLLLLLGGGS

SEQ ID NO: 238 - protein, 16 aa, synthetic construct
SGGGAAAAAAAAGGGS

SEQ ID NO: 239 - protein, 24 aa, synthetic construct
SGGGAAAAAAAAAAAAAAAAGGGS

SEQ ID NO: 240 - protein, 24 aa, synthetic construct
SGALGGLALAGLLLAGLGLGAAGS

SEQ ID NO: 241 - protein, 14 aa, synthetic construct
SLSLSPGGGGGPAR

SEQ ID NO: 242 - protein, 25 aa, synthetic construct
SLSLSPGGGGGPARSLSLSPGGGGG

SEQ ID NO: 243 - protein, 6 aa, synthetic construct
GSSGSS

SEQ ID NO: 244 - protein, 7 aa, synthetic construct
GSSSSSS

SEQ ID NO: 245 - protein, 6 aa, synthetic construct
GGSSSS

SEQ ID NO: 246 - protein, 6 aa, synthetic construct
GSSSSS

SEQ ID NO: 247 - protein, 6 aa, synthetic construct
SGGGGS

SEQ ID NO: 248 - protein, 12 aa, synthetic construct
KLEEGEFSEARV

SEQ ID NO: 249 - protein, 12 aa, synthetic construct
ILEEGEFSEAGC

SEQ ID NO: 250 - protein, 12 aa, synthetic construct
  VARIANT 1: note = K or I
  VARIANT 11: note = R or G
  VARIANT 12: note = V or C
XLEEGEFSEAXX

SEQ ID NO: 251 - protein, 55 aa, synthetic construct
FVPVFLPAKPTTTPAPRPPTPAPTIASQPLSLRPEACRPAAGGAVHTRGLDFACD

SEQ ID NO: 252 - protein, 54 aa, synthetic construct
FVPVFLPAKPTTTPAPRPPTPAPTIASQPLSLRPEASRPAAGGAVHTRGLDFAD

SEQ ID NO: 253 - protein, 58 aa, synthetic construct
FSSVVPVLQKVNSTTTKPVLRTPSPVHPTGTSQPQRPEDCRPRGSVKGTGLDFACDIY

SEQ ID NO: 254 - DNA, 174 bp, synthetic construct
  source 1..174: mol_type = other DNA
ttcagctctgtggtgcccgtgctgcagaaagtgaacagcaccaccacaaagcctgtgctgagaaccccttctcctgtgcaccctaccggcacaagccagcctcaaagacctgaggactgcagacctagaggctccgtgaaaggcacaggcctggacttcgcctgcgacatctat

SEQ ID NO: 255 - protein, 21 aa, Homo sapiens
IYIWAPLAGTCGVLLLSLVIT

SEQ ID NO: 256 - protein, 81 aa, synthetic construct
FVPVFLPAKPTTTPAPRPPTPAPTIASQPLSLRPEASRPAAGGAVHTRGLDFADIYIWAPLAGTCGVLLLSLVITLYCNHR

SEQ ID NO: 257 - protein, 82 aa, synthetic construct
FVPVFLPAKPTTTPAPRPPTPAPTIASQPLSLRPEACRPAAGGAVHTRGLDFACDIYIWAPLAGTCGVLLLSLVITLYCNHR

SEQ ID NO: 258 - protein, 219 aa, Homo sapiens
PCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 231 aa, Homo sapiens
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 261 - protein, 22 aa, Homo sapiens
MALIVLGGVAGLLLFIGLGIFF

SEQ ID NO: 262 - protein, 21 aa, synthetic construct
MALPVTALLLPLALLLHAAAP

SEQ ID NO: 263 - protein, 21 aa, synthetic construct
MALPVTALLLPLALLLHAARP

SEQ ID NO: 264 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 43 aa, Homo sapiens
WVRSKRSRLLHSDYMNMTPRRPGPTRKHYQPYAPPRDFAAYRS

SEQ ID NO: 266 - protein, 42 aa, synthetic construct
ALYLLRRDQRLPPDAHKPPGGGSFRTPIQEEQADAHSTLAKI

SEQ ID NO: 267 - protein, 42 aa, synthetic construct
KRGRKKLLYIFKQPFMRPVQTTQEEDGCSCRFPEEEEGGCEL

SEQ ID NO: 268 - protein, 38 aa, synthetic construct
CWLTKKKYSSSVHDPNGEYMFMRAVNTAKKSRLTDVTL

SEQ ID NO: 269 - protein, 197 aa, synthetic construct
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 270 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 271 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 272 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 273 - protein, 208 aa, synthetic construct
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 274 - DNA, 2535 bp, Homo sapiens
  source 1..2535: mol_type = genomic DNA
gaattccggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctcgtccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgaggtaattctgcacgtttgcccgcgtgctctctgtgctctgtcgcaaacctctttggccggagccgacttgttctctcgtctgctctaagttcttttagcttttggctggccccaggggtcctaggcttggagggcaggctgctcacgggtttggtggtttctagccgcattttccccagcccagaaagcacccgaagtcaccttcggggatggatccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattg